(19) United States
(12) Patent Application Publication
(10) Pub. No.: US 2024/0093189 A1
(43) Pub. Date: Mar. 21, 2024

(54) VECTORS COMPRISING STUFFER POLYNUCLEOTIDE SEQUENCES

(71) Applicant: Maze Therapeutics, Inc., South San Francisco, CA (US)

(72) Inventors: Carleton Proctor GOOLD, Menlo Park, CA (US); Robert R. GRAHAM, San Francisco, CA (US); Peter JANKI, San Francisco, CA (US); Ronald CHEN, Pacifica, CA (US); Eric GREEN, South San Francisco, CA (US)

(21) Appl. No.: 18/263,118

(22) PCT Filed: Feb. 4, 2022
(86) PCT No.: PCT/US2022/015380

§ 371 Date: Jul. 26, 2023

Related U.S. Application Data
(60) Provisional application No. 63/146,522, filed on Feb. 5, 2021.

(30) Foreign Application Priority Data
Feb. 5, 2021 (WO) PCT/US2021/016939

(51) Int. Cl. C12N 15/113; C12N 15/86

(52) U.S. Cl. CPC C12N 15/113; C12N 15/86; C12N 2310/141; C12N 2320/32; C12N 2750/14143; C12N 2830/38

(57) ABSTRACT

The present disclosure provides vector stuffer polynucleotides and compositions thereof, including expression constructs and vectors, such as viral vectors and methods of delivering a therapeutic agent (e.g., inhibitory nucleic acid) to a mammal or treating a disease.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 630264_402WO_SEQUENCE_LISTING.txt. The text file is 163 KB, was created on Feb. 4, 2022, and is being submitted electronically via EFS-Web.

### BACKGROUND

The size of the adeno-associated virus (AAV) vector genome is known to be constrained, with vector sizes of approximately the native size of 4.7 kb being packaged successfully but much larger vector genomes subject to reduced production of functional AAV vector (Wu et al., Molecular Therapy 2010 18(1): 80-86). On the other hand, vector genomes which are substantially smaller than the vector genome packaging limit can result in suboptimal packaging (Dong et al., Human gene therapy 1996 7:2101-2112). Also, if the cis plasmid has a ‘backbone’ sequence—such as typically encoding the elements of bacterial origins of resistance and antibiotic resistance, needed to propagate the plasmid in bacteria—that is close in size to the intended AAV vector genome, the amount of unintended ‘reverse packaged’ sequence can increase (Hauck et al., Molecular Therapy (2009) 17:144-152). For these reasons, it may be beneficial to include ‘stuffer sequences’ that on their own do not confer unfavorable properties to packaged AAV, as material in the plasmid backbone, and for use in situations where the intended AAV payload is considerably shorter than the native AAV packaging size. The importance of inert stuffer sequence has been highlighted by Keiser et al., Nature Medicine (2021) 27:1982-1989, whereby it was shown that a ‘payload-free’ AAV with stuffer sequence was capable of inducing substantial toxicity in non-human primates.

There is a current need for AAV vectors having improved packaging features.

### BRIEF DESCRIPTION OF THE SEVERAL VIEWS OF THE DRAWINGS

FIG. 1: Tracks selected on UCSC genome browser to identify expressed regions, known or predicted regulatory elements and repetitive elements (including retroviral and transposable elements) within the base sequence.

FIGS. 2A-2B show the liver enzyme function tests (FIG. 2A) aspartate transaminase (AST; U/L) and (FIG. 2B) alanine transaminase (ALT; U/L) measured 13-days post-dosing with processed serum of mice dosed intravenously with vehicle or scAAV9 vectors. When compared to control mice, no stuffer only vectors had a significant elevation in either ALT or AST whereas the vector expression in the toxic positive control artificial miRNA did elicit a substantial and significant rise in liver enzymes ALT and AST (one-way ANOVA, Dunnett's multiple comparisons test). All groups were N=5 except for the toxic positive control (N=3).

FIGS. 3A-3H: show Fragment Analyzer traces of column purified scAAV9 vector DNA. Using a representative vector, FIG. 3A shows the standard high-molecular weight double peak, which upon enzymatic cleavage of the mutITR (double peak retained; FIG. 3B), or wtITRs (now single peak; FIG. 3C) demonstrates that the double-peak represents the full-length population. FIG. 3D labels the 3 components of the Fragment Analyzer trace of purified scAAV9 vector DNA: full-length vector as represented by the two peaks at the highest molecular weight, the next peak, or miR-centered truncations, and lastly truncations occurring within the stuffer sequence. In FIG. 3E are the traces for scAAV9 H1 MCS constructs PSG11_V1 and PSG11_V2 in the top trace and bottom trace, respectively. In the trace shown in FIG. 3F, when overlapped, PSG11_V2 has a higher concentration of truncations compared to PSG11_V1. In FIG. 3G are Fragment Analyzer traces of PSG11_V1 vector (top) and PSG11_V2 vector (bottom) embedded with the artificial miRNA miR-1-1 XD-14792 (XD-14792 is also referred to herein as 1784). FIG. 3H shows overlap of the traces in FIG. 3G. As evidenced by the size of the miR-centered peak (arrow), the miR-centered truncation is reduced in PSG11_V1 compared to PSG11_V2.

FIGS. 4A-4B show the predicted secondary structure of 200 nucleotides adjacent to the artificial miRNA and terminator of PSG11_V1 (FIG. 4A) and PSG11_V2 (FIG. 4B) using Mfold web server. As evidenced by a less negative gibbs free energy value and a relative lack of secondary structure, PSG11_V1 compared to PSG11_V2 has a more desirable predicted DNA secondary structure adjacent to artificial miRNA.

FIGS. 5A-5B show liver enzyme values (FIG. 5A—AST; FIG. 5B—ALT) of the vector architecture study.

FIG. 6 shows stranded RNAseq traces from representative vectors with H1 long promoter, amiRNA 3330 in miR-100 backbone and PSG11_V5 stuffer (left) and H1 short promoter, amRNA 1784 (also referred to herein 14792) in miR-1-1 backbone and PSG11_V5 stuffer (right).

FIG. 7: Shows the titers of the 24 constructs tested in the vector architecture study. Vectors containing the H1 long promoter (left column), had consistently higher titers compared to both H1 native promoter (middle) and H1 short promoter (right).

FIGS. 8A-8C show miR and stuffer truncations for the scAAV9 vectors with AMELY_V3 and PSG11_V5 stuffer sequences as measured by Fragment Analyzer parallel capillary electrophoresis system. In FIG. 8A, across all 3 promoter contexts, H1 long (left panel), H1 native (middle panel), and H1 short (right panel), and both without (circle) and with an SV40 polyadenylation sequence (triangle), miR100 has fewer miR truncations compared to miR1-1 in both AMELY_V3 and PSG11_V5 vectors. In FIG. 8B, the vector with H1 long promoter, both with and without an SV40 polyadenylation sequence, has fewer stuffer truncations compared to vectors with H1 native promoter and H1 short promoter. In FIG. 8C, the vector with H1 long promoter, both with and without an SV40 polyadenylation sequence, has fewer truncations in the combined miR/stuffer truncations compared to vector with H1 native promoter and vector with H1 short promoter.

### DETAILED DESCRIPTION

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “nucleic acid” or “polynucleotide” refer to any nucleic acid polymer composed of covalently linked nucleotide subunits, such as polydeoxyribonucleotides or polyribonucleotides. Examples of nucleic acids include RNA and DNA.

As used herein, “RNA” refers to a molecule comprising one or more ribonucleotides and includes double-stranded RNA, single-stranded RNA, isolated RNA, synthetic RNA, recombinant RNA, as well as modified RNA that differs from naturally-occurring RNA by the addition, deletion, substitution, and/or alternation of one or more nucleotides. Nucleotides of RNA molecules may comprise standard nucleotides or non-standard nucleotides, such as non-naturally occurring nucleotides or chemically synthesized nucleotides.

As used herein, “DNA” refers to a molecule comprising one or more deoxyribonucleotides and includes double-stranded DNA, single-stranded DNA, isolated DNA, synthetic DNA, recombinant DNA, as well as modified DNA that differs from naturally-occurring DNA by the addition, deletion, substitution, and/or alteration of one or more nucleotides. Nucleotides of DNA molecules may comprise standard nucleotides or non-standard nucleotides, such as non-naturally occurring nucleotides or chemically synthesized nucleotides.

“Isolated” refers to a substance that has been isolated from its natural environment or artificially produced. As used herein with respect to a cell, “isolated” refers to a cell that has been isolated from its natural environment (e.g., from a subject, organ, tissue, or bodily fluid). As used herein with respect to a nucleic acid, “isolated” refers to a nucleic acid that has been isolated or purified from its natural environment (e.g., from a cell, cell organelle, or cytoplasm), recombinantly produced, amplified, or synthesized. In embodiments, an isolated nucleic acid includes a nucleic acid contained within a vector.

As used herein, the term “wild-type” or “non-mutant” form of a gene refers to a nucleic acid that encodes a protein associated with normal or non-pathogenic activity (e.g., a protein lacking a mutation, such as a repeat region expansion that results in higher risk of developing, onset, or progression of a neurodegenerative disease).

As used herein, the term “mutation” refers to any change in the structure of a gene, e.g., gene sequence, resulting in an altered form of the gene, which may be passed onto subsequent generations (hereditary mutation) or not (somatic mutation). Gene mutations include the substitution, insertion, or deletion of a single base in DNA or the substitution, insertion, deletion, or rearrangement of multiple bases or larger sections of genes or chromosomes, including repeat expansions.

As used herein, the term “inhibitory nucleic acid” refers to a nucleic acid that comprises a guide strand sequence that hybridizes to at least a portion of a target nucleic acid, e.g., neurodenerative disease target RNA, mRNA, pre-mRNA, or mature mRNA, and inhibits its expression or activity. An inhibitory nucleic acid may target a protein coding region (e.g., exon) or non-coding region (e.g., 5′UTR, 3′UTR, intron, etc.) of a target nucleic acid. In some embodiments, an inhibitory nucleic acid is a single stranded or double stranded molecule. An inhibitory nucleic acid may further comprise a passenger strand sequence on a separate strand (e.g., double stranded duplex) or in the same strand (e.g., single stranded, self-annealing duplex structure). In some embodiments, an inhibitory nucleic acid is an RNA molecule, such as a siRNA, shRNA, miRNA, or dsRNA.

As used herein, a “microRNA” or “miRNA” refers to a small non-coding RNA molecule capable of mediating silencing of a target gene by cleavage of the target mRNA, translational repression of the target mRNA, target mRNA degradation, or a combination thereof. Typically, miRNA is transcribed as a hairpin or stem-loop (e.g., having a self-complementary, single-stranded backbone) duplex structure, referred to as a primary miRNA (pri-miRNA), which is enzymatically processed (e.g., by Drosha, DGCR8, Pasha, etc.) into a pre-miRNA. Pre-miRNA is exported into the cytoplasm, where it is enzymatically processed by Dicer to produce a miRNA duplex with the passenger strand and then a single-stranded mature miRNA molecule, which is subsequently loaded into the RNA-induced silencing complex (RISC). Reference to a miRNA may include synthetic or artificial miRNAs.

As used herein, a “synthetic miRNA” or “artificial miRNA” or “amiRNA” refers to an endogenous, modified, or synthetic pri-miRNA or pre-miRNA (e.g., miRNA backbone or scaffold) in which the endogenous miRNA guide sequence and passenger sequence within the stem sequence have been replaced with a miRNA guide sequence and a miRNA passenger sequence that direct highly efficient RNA silencing of the targeted gene (see, e.g., Eamens et al., Methods Mol. Biol. (2014) 1062:211-224). In some embodiments, the nature of the complementarity of the guide and passenger sequences (e.g., number of bases, position of mismatches, types of bulges, etc.) can be similar or different from the nature of complementarity of the guide and passenger sequences in the endogenous miRNA backbone upon which the synthetic miRNA is constructed.

As used herein, the term “microRNA backbone,” “miR backbone,” “microRNA scaffold,” or “miR scaffold” refers to a pri-miRNA or pre-miRNA scaffold, with the stem sequence replaced by a miRNA of interest, and is capable of producing a functional, mature miRNA that directs RNA silencing at the gene targeted by the miRNA of interest. A miR backbone comprises a 5′ flanking region (also referred to 5′ miR context, >9 nucleotides), a stem region comprising the miRNA duplex (guide strand sequence and passenger strand sequence) and basal stem (5′ and 3′, each about 4-13 nucleotides), at least one loop motif region including the terminal loop (>10 nucleotides for terminal loop), a 3′ flanking region (also referred to 3′ miR context, >9 nucleotides), and optionally one or more bulges in the stem. A miR backbone may be derived completely or partially from a wild type miRNA scaffold or be a completely artificial sequence.

As used herein, the term “antisense strand sequence” or “guide strand sequence” of an inhibitory nucleic acid refers to a sequence that is substantially complementary (e.g., at least 45%, 50%, 55%, 60%, 65%, 70%, 75%, 80%, 85%, 90%, 95%, or 100% complementary) to a region of about 10-50 nucleotides (e.g., about 15-30, 16-25, 18-23, or 19-22 nucleotides) of the mRNA of the gene targeted for silencing. The antisense sequence is sufficiently complementary to the target mRNA sequence to direct target-specific silencing, e.g., to trigger the destruction of the target mRNA by the RNAi machinery or process. In some embodiments, the antisense sequence or guide strand sequence refers to the mature sequence remaining following cleavage by Dicer.

As used herein, the term “sense sequence” or “passenger strand sequence” of an inhibitory nucleic acid refers to a sequence that is homologous to the target mRNA and partially or completely complementary to the antisense strand sequence or guide strand sequence of an inhibitory nucleic acid. The antisense strand sequence and sense strand sequence of an inhibitory nucleic acid are hybridized to form a duplex structure (e.g., forming a double-stranded duplex or single-stranded self-annealing duplex structure). In some embodiments, the sense sequence or passenger strand sequence refers to the mature sequence remaining following cleavage by Dicer.

As used herein, a “duplex,” when used in reference to an inhibitory nucleic acid, refers to two nucleic acid strands (e.g., a guide strand and passenger strand) hybridizing together to form a duplex structure. A duplex may be formed by two separate nucleic acid strands or by a single nucleic acid strand having a region of self-complementarity (e.g., hairpin or stem-loop).

As used herein, the term “complementary” refers to the ability of polynucleotides to form base pairs with each other. Base pairs are typically formed by hydrogen bonds between nucleotide subunits in antiparallel polynucleotide strands or a single, self-annealing polynucleotide strand. Complementary polynucleotide strands can form base pairs in the Watson-Crick manner (e.g., A to T, A to U, C to G), or in any other manner that allows for the formation of duplexes. As apparent to skilled persons in the art, when using RNA as opposed to DNA, uracil rather than thymine is the base that is considered to be complementary to adenosine. Furthermore, when a “U” is denoted in the context of the present invention, the ability to substitute a “T” is understood, unless otherwise stated. Complementarity also encompasses Watson-Crick base pairing between non-modified and modified nucleobases (e.g., 5-methyl cytosine substituted for cytosine). Full complementarity, perfect complementarity or 100% complementarity between two polynucleotide strands is where each nucleotide of one polynucleotide strand can form hydrogen bond with a nucleotide unit of a second polynucleotide strand. % complementarity refers to the number of nucleotides of a contiguous nucleotide sequence in a nucleic acid molecule that are complementary to an aligned reference sequence (e.g., a target mRNA, passenger strand), divided by the total number of nucleotides and multiplying by 100. In such an alignment, a nucleobase/nucleotide which does not form a base pair is called a mismatch. Insertions and deletions are not permitted in calculating % complementarity of a contiguous nucleotide sequence. It is understood by skilled persons in the art that in calculating complementarity, chemical modifications to nucleobases are not considered as long as the Watson-Crick base pairing capacity of the nucleobase is retained (e.g., 5-methyl cytosine is considered the same as cytosine for the purpose of calculating % complementarity).

The “percent identity” between two or more nucleic acid sequences refers to the proportion nucleotides of a contiguous nucleotide sequence in a nucleic acid molecule that are shared by a reference sequence (i.e., % identity=number of identical nucleotides/total number of nucleotides in the aligned region (e.g., the contiguous nucleotide sequence)×100). Insertions and deletions are not permitted in the calculation of % identity of a contiguous nucleotide sequence. It is understood by skilled persons in the art that in calculating identity, chemical modifications to nucleobases are not considered as long as the Watson-Crick base pairing capacity of the nucleobase is retained (e.g., 5-methyl cytosine is considered the same as cytosine for the purpose of calculating % identity).

As used herein, the term “hybridizing” or “hybridizes” refers to two nucleic acids strands forming hydrogen bonds between base pairs on antiparallel strands, thereby forming a duplex. The strength of hybridization between two nucleic acid strands may be described by the melting temperature (Tₘ), defined as at a given ionic strength and pH, the temperature at which 50% of a target sequence hybridizes to a complementary polynucleotide.

As used herein, “expression construct” refers to any type of genetic construct containing a nucleic acid (e.g., transgene) in which part or all of the nucleic acid encoding sequence is capable of being transcribed. In some embodiments, expression includes transcription of the nucleic acid, for example, to generate a biologically-active polypeptide product or inhibitory RNA (e.g., siRNA, shRNA, miRNA) from a transcribed gene. In some embodiments, the transgene is operably linked to expression control sequences.

As used herein, the term “transgene” refers to an exogenous nucleic acid that has been transferred naturally or by genetic engineering means into another cell and is capable of being transcribed, and optionally translated.

As used herein, the term “gene expression” refers to the process by which a nucleic acid is transcribed from a nucleic acid molecule, and often, translated into a peptide or protein. The process can include transcription, post-transcriptional control, post-transcriptional modification, translation, post-translational control, post translational modification, or any combination thereof. Reference to a measurement of “gene expression” may refer to measurement of the product of transcription (e.g., RNA or mRNA), the product of translation (e.g., peptides or proteins).

As used herein, the term “inhibit expression of a gene” means to reduce, down-regulate, suppress, block, lower, or stop expression of the gene. The expression product of a gene can be a RNA molecule transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

As used herein, “vector” refers to a genetic construct that is capable of transporting a nucleic acid molecule (e.g., transgene encoding inhibitory nucleic acid) between cells and effecting expression of the nucleic acid molecule when operably-linked to suitable expression control sequences. Expression control sequences may include transcription initiation sequence, termination sequence (also referred to herein as terminator sequence), promoter sequence and enhancer sequence; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. The vector may be a plasmid, phage particle, transposon, cosmid, phagemid, chromosome, artificial chromosome, virus, virion, etc. Once transformed into a suitable host cell, the vector may replicate and function independently of the host genome, or may, in some instances, integrate into the genome itself.

As used herein, “host cell” refers to any cell that contains, or is capable of containing a composition of interest, e.g., an inhibitory nucleic acid. In embodiments, a host cell is a mammalian cell, such as a rodent cell, (mouse or rat) or primate cell (monkey, chimpanzee, or human). In embodiments, a host cell may be in vitro or in vivo. In embodiments, a host cell may be from an established cell line or primary cells. In embodiments, a host cell is a cell of the CNS, such as a neuron, glial cell, astrocyte, and microglial cell.

As used herein, “neurodegenerative disease” or “neurodegenerative disorder” refers to diseases or disorders that exhibit neural cell death as a pathological state. A neurodegenerative disease may exhibit chronic neurodegeneration, e.g., slow, progressive neural cell death over a period of several years, or acute neurodegeneration, e.g., sudden onset or neural cell death. Examples of chronic, neurodegenerative diseases include Alzheimer's disease, Parkinson's disease, Huntington's disease, spinocerebellar ataxia type 2 (SCA2), frontotemporal dementia (FTD), and amyotrophic lateral schlerosis (ALS). Chronic neurodegenerative diseases include diseases that feature TDP-43 proteinopathy, which is characterized by nucleus to cytoplasmic mislocalization, deposition of ubiquitinated and hyper-phosphorylated TDP-43 into inclusion bodies, protein truncation leading to formation of toxic C-terminal TDP-43 fragments, and protein aggregation. TDP-43 proteinopathy diseases include ALS, FTD, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, and Alzheimer's disease. Acute neurodegeneration may be caused by ischemia (e.g., stroke, traumatic brain injury), axonal transection by demyelination or trauma (e.g., spinal cord injury or multiple sclerosis). A neurodegenerative disease may exhibit death of mainly one type of neuron or of multiple types of neurons.

As used herein, “subject,” “patient,” and “individual” are used interchangeably herein and refer to living organisms (e.g., mammals) selected for treatment or therapy. Examples of subjects include human and non-human mammals, such as primates (monkey, chimpanzee), cows, horses, sheep, dogs, cats, rats, mice, guinea pigs, pigs, and transgenic species thereof.

### Stuffer Sequences and Expression Constructs

AAV preferentially packages a full-length genome, i.e., one that is approximately the same size as the native genome, and is not too big or too small. However, expression cassettes encoding inhibitory nucleic acid sequences are substantially smaller than AAV full-length genome. To avoid packaging of fragmented genomes, a stuffer sequence may be linked to an expression construct comprising a heterologous nucleic acid sequence and flanked by the 5′ ITR and 3′ ITR to expand the packagable genome, resulted in a genome whose size was near-normal in length between the ITRs. Generally, the packaging capacity of AAV is about 4.7 kb between the 5′ ITR and 3′ ITR. For self-complementary AAV (scAAV) vector, the packaging capacity is about 2.4 kb between the 5′ ITR and 3′ ITR.

Preferably, the starting sequence for obtaining a vector stuffer sequence is of mammalian origin, such as human origin. The length of the stuffer sequence may be adjusted such that the vector genome is at or close to the (natural) packaging limit of AAV capsid. Furthermore, a vector stuffer sequence can be designed to minimize adverse effects in the context of in vivo gene therapy. For example, regions of the human genome may be identified as a source for vector stuffer sequences by identifying sequences with minimal impact if integration in the genome occurs and minimal risk of initiating unexpected transcription. Therefore, regions of the genome may be examined in which i) deletions and duplications were common in the population and not associated with disease-relevant phenotypes (no evidence of evolutionary pressure) and/or ii) RNA expression across human tissues was low or undetectable (lack strong intrinsic enhancers/promoter elements). Furthermore, vector stuffer sequences can be designed to have reduced, minimized, removed, or to lack one or more elements to make the vector sequence inert or safe. In some embodiments, the vector stuffer sequence is modified to: reduce or remove expressed regions (e.g., exons+10 bp on either side, human ESTs); reduce or remove regulatory elements (e.g., promoter sequences, enhancer sequences, repressor sequences, splicing donors or acceptors, or other cis-acting elements found in the human genome that could potentially affect transcription of the transgene); reduce or remove repetitive elements (e.g., microsatellite repeats, dinucleotides repeats, trinucleotide repeats); reduce, remove, or modify ATG codons to reduce or eliminate the possibility of peptides being generated from the filler or stuffer sequence due to latent start codons; reduce or remove CpG dinucleotides to lower likelihood of unmethylated CpG dinucleotides (from cis-plasmids generated in bacteria) inducing an innate immune response; or any combination thereof. The present disclosure provides vector stuffer sequences possessing one or more of the aforementioned features, with further advantages of one or more of: high titer; low toxicity; and reduced truncations in miRNA and/or stuffer sequence.

In one aspect, the present disclosure provides a vector stuffer sequence comprising a nucleic acid of about 1300 to about 2300 nucleotides in length and having at least 75% identity to: nucleotides 489-2185 of any one of SEQ ID NOS:13-16; nucleotides 342-2222 of SEQ ID NO:17; nucleotides 488-2177 of SEQ ID NO: 18; nucleotides 489-2177 of SEQ ID NO:19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51.

In some embodiments, the vector stuffer sequence comprises a nucleic acid of about 1500-2000 nucleotides in length and having at least 75% identity to: nucleotides 489-2185 of any one of SEQ ID NOS:13-16; nucleotides 342-2222 of SEQ ID NO:17; nucleotides 488-2177 of SEQ ID NO:18; nucleotides 489-2177 of SEQ ID NO:19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51.

In some embodiments, the vector stuffer sequence comprises a nucleic acid of about 1600 to 1900 nucleotides in length and having at least 75% identity to: nucleotides 489-2185 of any one of SEQ ID NOS:13-16; nucleotides 342-2222 of SEQ ID NO:17; nucleotides 488-2177 of SEQ ID NO: 18; nucleotides 489-2177 of SEQ ID NO: 19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51.

In some embodiments, the vector stuffer sequence comprises a nucleic acid having at least 80% identity to: nucleotides 489-2185 of any one of SEQ ID NOS:13-16; nucleotides 342-2222 of SEQ ID NO:17; nucleotides 488-2177 of SEQ ID NO:18; nucleotides 489-2177 of SEQ ID NO:19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51. In some embodiments, the vector stuffer sequence comprises a nucleic acid of about 1,300-2,300 nucleotides in length, about 1,500-2,000 nucleotides in length, or about 1,600-1,900 in length.

In some embodiments, the vector stuffer sequence comprises a nucleic acid having at least 85% identity to: nucleotides 489-2185 of any one of SEQ ID NOS:13-16; nucleotides 342-2222 of SEQ ID NO:17; nucleotides 488-2177 of SEQ ID NO:18; nucleotides 489-2177 of SEQ ID NO:19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51. In some embodiments, the vector stuffer sequence comprises a nucleic acid of about 1,300-2,300 nucleotides in length, about 1,500-2,000 nucleotides in length, or about 1,600-1,900 in length.

In some embodiments, the vector stuffer sequence comprises a nucleic acid having at least 90% identity to: nucleotides 489-2185 of any one of SEQ ID NOS:13-16; nucleotides 342-2222 of SEQ ID NO:17; nucleotides 488-2177 of SEQ ID NO:18; nucleotides 489-2177 of SEQ ID NO:19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51. In some embodiments, the vector stuffer sequence comprises a nucleic acid of about 1,300-2,300 nucleotides in length, about 1,500-2,000 nucleotides in length, or about 1,600-1,900 in length.

In some embodiments, the vector stuffer sequence comprises a nucleic acid having at least 95% identity to: nucleotides 489-2185 of any one of SEQ ID NOS:13-16; nucleotides 342-2222 of SEQ ID NO:17; nucleotides 488-2177 of SEQ ID NO:18; nucleotides 489-2177 of SEQ ID NO:19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51. In some embodiments, the vector stuffer sequence comprises a nucleic acid of about 1,300-2,300 nucleotides in length, about 1,500-2,000 nucleotides in length, or about 1,600-1,900 in length. In some embodiments, the vector stuffer sequence comprises a nucleic acid having at least 97% identity to: nucleotides 489-2185 of any one of SEQ ID NOS:13-16; nucleotides 342-2222 of SEQ ID NO:17; nucleotides 488-2177 of SEQ ID NO:18; nucleotides 489-2177 of SEQ ID NO: 19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51. In some embodiments, the vector stuffer sequence comprises a nucleic acid of about 1,300-2,300 nucleotides in length, about 1,500-2,000 nucleotides in length, or about 1,600-1,900 in length.

In some embodiments, the vector stuffer sequence comprises or consists of: nucleotides 489-2185 of any one of SEQ ID NOS:13-16; nucleotides 342-2222 of SEQ ID NO:17; nucleotides 488-2177 of SEQ ID NO: 18; nucleotides 489-2177 of SEQ ID NO: 19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO: 80; nucleotides 342-2222 of SEQ ID NO:81; SEQ ID NO:45; SEQ ID NO:46; SEQ ID NO:47; SEQ ID NO:48; SEQ ID NO:49, SEQ ID NO:50; or SEQ ID NO:51.

In some embodiments, the vector stuffer sequence comprises or consists of SEQ ID NO:48 or nucleotides 489-2185 of any one of SEQ ID NOS:13-16.

In some embodiments, the vector is an adeno-associated virus (AAV) vector, optionally wherein the AAV vector is self-complementary.

In some embodiments, the vector stuffer sequence is positioned adjacent to (e.g., 5′ or 3′) an expression construct comprising a heterologous nucleic acid sequence. In some embodiments, the heterologous nucleic acid sequence encodes a therapeutic agent. In some embodiments, the therapeutic agent comprises a nucleic acid encoding a therapeutic protein or an inhibitory nucleic acid. In some embodiments, the inhibitory nucleic acid comprises a siRNA, miRNA, shRNA, or dsRNA. In some embodiments, the inhibitory nucleic acid comprises a siRNA, miRNA, shRNA, of dsRNA targeting a neurodegenerative disease related gene. In some embodiments, the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease. In some embodiments, the neurodegenerative disease is a polyglutamine repeat disease. In some embodiments, the inhibitory nucleic acid (e.g., siRNA, miRNA, shRNA, or dsRNA) targets ATXN2. ATXN2 refers to a protein encoded by the ATXA2 gene, which contains a polyglutamine (polyQ, CAG repeat) tract. ATXA2 gene or transcript may refer to normal alleles of ATXN2, which typically have 22 or 23 repeats, or mutated alleles having intermediate (˜24-32 repeats) or longer repeat expansions (˜33 to >100 repeats). In some embodiments, ATXA2 refers to mammalian ATNX2, including human ATXN2.

In some embodiments, the expression construct comprises a heterologous nucleic acid encoding an artificial miRNA targeting ATXN2. Examples of heterologous nucleic acids encoding guide sequences and passenger sequences and artificial miRNAs targeting ATXN2 are provided in Table 5. RNA formats of the encoded guide, passenger, and artificial miRNA sequences are provided, as well as the DNA formats for insertion into cis plasmids and rAAV vectors. In some embodiments, a heterologous nucleic acid encodes an artificial miRNA targeting ATXN2 and comprises a guide sequence selected from SEQ ID NOS:1-4 and 71. In some embodiments, a heterologous nucleic acid encodes an artificial miRNA comprising a guide sequence provided by SEQ ID NO:1 and a passenger sequence provided by SEQ ID NO:5. In some embodiments, a heterologous nucleic acid comprises a guide sequence provided by SEQ ID NO:2 and a passenger sequence provided by SEQ ID NO:6. In some embodiments, a heterologous nucleic acid comprises a guide sequence provided by SEQ ID NO:3 and a passenger sequence provided by SEQ ID NO:7. In some embodiments, a heterologous nucleic acid comprises a guide sequence provided by SEQ ID NO:4 and a passenger sequence provided by SEQ ID NO:8. In some embodiments, a heterologous nucleic acid comprises a guide sequence provided by SEQ ID NO:71 and a passenger sequence provided by SEQ ID NO:72. In some embodiments, the heterologous nucleic acid comprises a guide sequence and passenger sequence embedded in a miRNA backbone (or scaffold). In some embodiments, the miRNA backbone is miR-100 or miR-1-1. In some embodiments, the heterologous nucleic acid encodes an artificial miRNA sequence provided by any one of SEQ ID NOs:9-12 and 73. Additional examples of ATXN2 targeting guide sequences, passenger sequences, miR backbones, and artificial miRNA sequences are provided in PCT Publication WO2021/159008, which is incorporated by reference in its entirety.

In some embodiments, the expression construct further comprises one or more expression control sequences (regulatory sequences) operably linked with the transgene (e.g., nucleic acid encoding an artificial miRNA). “Operably linked” sequences include expression control sequences that are contiguous with the transgene or act in trans or at a distance from the transgene to control its expression. Examples of expression control sequences include transcription initiation sequences, termination sequences, promoter sequences, enhancer sequences, repressor sequences, splice site sequences, polyadenylation (polyA) signal sequences, or any combination thereof.

In some embodiments, a promoter is an endogenous promoter, synthetic promoter, constitutive promoter, inducible promoter, tissue-specific promoter (e.g., CNS-specific), or cell-specific promoter (neurons, glial cells, or astrocytes). Examples of constitutive promoters include, Rous sarcoma virus (RSV) LTR promoter (optionally with the RSV enhancer), cytomegalovirus (CMV) promoter (optionally with the CMV enhancer), SV40 promoter, and dihydrofolate reductase promoter. Examples of inducible promoters include zinc-inducible sheep metallothionine (MT) promoter, dexamethasone (Dex)-inducible mouse mammary tumor virus (MMTV) promoter, T7 polymerase promoter system, the ecdysone insect promoter, tetracycline-repressible system, tetracycline-inducible system, RU486-inducible system, and the rapamycin-inducible system. Further examples of promoters that may be used include, for example, chicken beta-actin promoter (CBA promoter), a CAG promoter, a H1 promoter, a CD68 promoter, a JeT promoter, synapsin promoter, RNA pol II promoter, or a RNA pol III promoter (e.g., U6, H1, etc.). In some embodiments, the promoter is a tissue-specific RNA pol II promoter. In some embodiments, the tissue-specific RNA pol II promoter is derived from a gene that exhibits neuron-specific expression. In some embodiments, the neuron-specific promoter is a synapsin 1 promoter or synapsin 2 promoter.

In some embodiments, the promoter is a H1 promoter. In some embodiments, the H1 promoter is promoter referred to herein as a “native” H1 promoter, such as a promoter comprising or consisting of the sequence set forth in SEQ ID NO:53. In some embodiments, the promoter is a promoter referred to herein as a “short” H1 promoter, such as a promoter comprising or consisting of the sequence set forth in SEQ ID NO:54 or nucleotides 113-203 of any one of SEQ ID NOS:17, 25-28, and 37-44. In some embodiments, the promoter is a promoter referred to herein as a “long” H1 promoter, such as a promoter comprising or consisting of the sequence set forth in SEQ ID NO:52 or nucleotides 113-343 of any one of SEQ ID NOS:13-16 and 18-24.

In some embodiments, the termination sequence is a SV40 termination sequence. Examples of SV40 termination sequence are set forth in SEQ ID NO:77; nucleotides 489-710 of any one of SEQ ID NOS:20, 21, 23, and 24; nucleotides 358-579 of any one of SEQ ID NOS:31, 32, 35, and 36; nucleotides 349-570 of any one of SEQ ID NOS:40, 41, and 44.

In some embodiments, the sequence encoding the inhibitory nucleic acid of the present disclosure is positioned in an untranslated region of an expression construct. In some embodiments, the sequence encoding the inhibitory nucleic acid of the present disclosure is positioned in an intron, a 5′ untranslated region (5′UTR), or a 3′ untranslated region (3′UTR) of the expression construct. In some embodiments, the sequence encoding the inhibitory nucleic acid of the present disclosure is positioned in an intron downstream of the promoter and upstream of an expressed gene.

### Vectors and Host Cells

In another aspect, provided are vectors comprising vector stuffer sequences of the present disclosure. A vector can be a plasmid, cosmid, phagemid, bacterial artificial chromosome (BAC) or viral vector. Examples of viral vectors include herpesvirus (HSV) vectors, retroviral vectors, adenoviral vectors, adeno-associated viral (AAV) vectors, lentiviral vectors, baculoviral vectors, and the like. In some embodiments, a retroviral vector is a mouse stem cell virus, murine leukemia virus (e.g., Moloney murine leukemia virus vector), feline leukemia virus, feline sarcoma virus, or avian reticuloendotheliosis virus vector. In some embodiments, a lentiviral vector is a HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV), equine infectious anemia virus, or Maedi-Visna viral vector.

In some embodiments, the vector is an adeno-associated virus (AAV) vector, such as a recombinant AAV (rAAV) vector, which is produced by recombinant methods. In some embodiments, the rAAV vector comprises the vector stuffer sequence of the present disclosure and an expression construct comprising a heterologous nucleic acid sequence (e.g., encoding an inhibitory nucleic acid). The vector stuffer sequence may be positioned adjacent to, either 5′ or 3′, the expression construct comprising the heterologous nucleic acid.

AAV is a single-stranded, non-enveloped DNA virus having a genome that encodes proteins for replication (rep) and the capsid (Cap), flanked by two ITRs, which serve as the origin of replication of the viral genome. AAV also contains a packaging sequence, allowing packaging of the viral genome into an AAV capsid. A recombinant AAV vector (rAAV) (also referred to as rAAV vector genome, or rAAV genome) may be obtained from the wild type genome of AAV by using molecular methods to remove the all or part of the wild type genome (e.g., Rep, Cap) from the AAV, and replacing it with a non-native nucleic acid, such as a heterologous nucleic acid sequence (e.g., a nucleic acid molecule encoding an inhibitory nucleic acid). Typically, for AAV one or both inverted terminal repeat (ITR) sequences are retained in the rAAV vector. In some embodiments, the rAAV vector comprises a 5′ inverted terminal repeat (ITR) and a 3′ ITR flanking the expression construct and vector stuffer sequence. Functional ITR sequences are necessary for the rescue, replication and packaging of the AAV viral particle. Thus, a rAAV vector is defined herein to include at least those sequences required in cis for replication and packaging (e.g., functional ITRs) of the virus. All other viral sequences may be supplied in trans. In some embodiments, the rAAV only retains the 5′ ITR and 3′ ITR from the AAV genome in order to maximize the size of the transgene that can be efficiently packaged by the vector. In some embodiments, each AAV ITR is a full length ITR (e.g., approximately 145 bp in length, and containing a functional Rep binding site (RBS) and a terminal resolution site (trs)). In some embodiments, one or both of the ITRs is is modified, e.g., by insertion, deletion, or substitution, provided that the ITRs provide for functional rescue, replication, and packaging. In some embodiments, a modified ITR lacks a functional terminal resolution site (trs) and is used for production of self-complementary AAV vectors (scAAV vectors). In some embodiments, the rAAV vector is a self-complementary AAV vector comprising a mutant ITR (lacking a terminal resolution site) on the 5′ side and a wild-type AAV ITR on the 3′ side. An example of a mutant 5′ ITR lacking a terminal resolution site is set forth in SEQ ID NO:57 or nucleotides 1-106 of any one of SEQ ID NOS:13-24, 29-44, and 78-82. In some embodiments, a modified ITR is a truncated version of AAV2 ITR referred to as AITR (D-sequence and TRS are deleted).

In some embodiments, the rAAV vector comprises a 5′ ITR comprising or consisting of SEQ ID NO:57 or nucleotides 1-106 of any one of SEQ ID NOS:13-24, 29-44, and 78-82. In some embodiments, the rAAV vector comprises a 3′ ITR comprising or consisting of SEQ ID NO:58, nucleotides 2192-2358 of any one of SEQ ID NOS:13-16, nucleotides 2214-2358 of any one of SEQ ID NOS:13-16, nucleotides 2229-2395 of SEQ ID NO:17, nucleotides 2251-2395 of SEQ ID NO:17, nucleotides 2184-2350 of SEQ ID NO:18, nucleotides 2206-2350 of SEQ ID NO:18; nucleotides 2206-2350 of SEQ ID NO:19; nucleotides 2216-2360 of SEQ ID NO:20; nucleotides 2216-2360 of SEQ ID NO:21; nucleotides 2206-2350 of SEQ ID NO:22; nucleotides 2216-2360 of SEQ ID NO:23; nucleotides 2216-2360 of SEQ ID NO:24; nucleotides 2161-2305 of SEQ ID NO:25; nucleotides 2161-2305 of SEQ ID NO:26; nucleotides 2161-2305 of SEQ ID NO:27; nucleotides 2161-2305 of SEQ ID NO:28; nucleotides 2266-2410 of SEQ ID NO:29; nucleotides 2224-2368 of SEQ ID NO:30; nucleotides 2216-2360 of SEQ ID NO:31; nucleotides 2225-2369 of SEQ ID NO:32; nucleotides 2266-2410 of SEQ ID NO:33; nucleotides 2224-2368 of SEQ ID NO:34; nucleotides 2216-2360 of SEQ ID NO:35; nucleotides 2225-2369 of SEQ ID NO:36; nucleotides 2257-2401 of SEQ ID NO:37; nucleotides 2258-2402 of SEQ ID NO:38; nucleotides 2215-2359 of SEQ ID NO:39; nucleotides 2207-2351 of SEQ ID NO:40; nucleotides 2207-2351 of SEQ ID NO:41; nucleotides 2257-2401 of SEQ ID NO:42; nucleotides 2215-2359 of SEQ ID NO:43; nucleotides 2207-2351 of SEQ ID NO:44; nucleotides 2258-2402 of SEQ ID NO:78; nucleotides 2267-2411 of SEQ ID NO:79; nucleotides 2251-2395 of SEQ ID NO:80; nucleotides 2251-2395 of SEQ ID NO:81; nucleotides 2187-2331 of SEQ ID NO:82. In some embodiments, the rAAV vector comprises: a 5′ ITR comprising or consisting of SEQ ID NO:57 and a 3′ ITR comprising or consisting of SEQ ID NO:58; a 5′ ITR comprising or consisting of nucleotides 1-106 of SEQ ID NO: 13 and a 3′ ITR comprising or consisting of nucleotides 2192-2358 of SEQ ID NO: 13; a 5′ ITR comprising or consisting of nucleotides 1-106 of SEQ ID NO:14 and a 3′ ITR comprising or consisting of nucleotides 2192-2358 of SEQ ID NO:14; a 5′ ITR comprising or consisting of nucleotides 1-106 of SEQ ID NO:15 and a 3′ ITR comprising or consisting of 2192-2358 of SEQ ID NO:15; a 5′ ITR comprising or consisting of nucleotides 1-106 of SEQ ID NO:16 and a 3′ ITR comprising or consisting of 2192-2358 of SEQ ID NO:16; a 5′ ITR comprising or consisting of nucleotides 1-106 of SEQ ID NO:17 and a 3′ ITR comprising or consisting of nucleotides 2229-2395 of SEQ ID NO:17; a 5′ ITR comprising or consisting of nucleotides 1-106 of SEQ ID NO:18 and a 3′ ITR comprising or consisting of nucleotides 2184-2350 of SEQ ID NO:18; a 5′ ITR comprising or consisting of nucleotides 1-106 of SEQ ID NO:13 and a 3′ ITR comprising or consisting of nucleotides 2214-2358 of SEQ ID NO:13; a 5′ ITR comprising or consisting of nucleotides 1-106 of SEQ ID NO:14 and a 3′ ITR comprising or consisting of nucleotides 2214-2358 of SEQ ID NO:14; a 5′ ITR comprising or consisting of nucleotides 1-106 of SEQ ID NO:15 and a 3′ ITR comprising or consisting of 2214-2358 of SEQ ID NO:15; a 5′ ITR comprising or consisting of nucleotides 1-106 of SEQ ID NO:16 and a 3′ ITR comprising or consisting of 2214-2358 of SEQ ID NO:16; a 5′ ITR comprising or consisting of nucleotides 1-106 of SEQ ID NO:17 and a 3′ ITR comprising or consisting of nucleotides 2251-2395 of SEQ ID NO:17; a 5′ ITR comprising or consisting of nucleotides 1-106 of SEQ ID NO:18 and a 3′ ITR comprising or consisting of nucleotides 2206-2350 of SEQ ID NO:18; a 5′ ITR comprising or consisting of nucleotides 1-106 of SEQ ID NO:19 and a 3′ ITR comprising or consisting of nucleotides 2206-2350 of SEQ ID NO:19; a 5′ ITR comprising or consisting of nucleotides 1-106 of SEQ ID NO:20 and a 3′ ITR comprising or consisting of nucleotides 2216-2360 of SEQ ID NO:20; a 5′ ITR comprising or consisting of nucleotides 1-106 of SEQ ID NO:21 and a 3′ ITR comprising or consisting of nucleotides 2216-2360 of SEQ ID NO:21; a 5′ ITR comprising or consisting of nucleotides 1-106 of SEQ ID NO:22 and a 3′ ITR comprising or consisting of nucleotides 2206-2350 of SEQ ID NO:22; a 5′ ITR comprising or consisting of nucleotides 1-106 of SEQ ID NO:23 and a 3′ ITR comprising or consisting of nucleotides 2216-2360 of SEQ ID NO:23; a 5′ ITR comprising or consisting of nucleotides 1-106 of SEQ ID NO:24 and a 3′ ITR comprising or consisting of nucleotides 2216-2360 of SEQ ID NO:24; a 5′ ITR comprising or consisting of nucleotides 1-106 of SEQ ID NO:25 and a 3′ ITR comprising or consisting of nucleotides 2161-2305 of SEQ ID NO:25; a 5′ ITR comprising or consisting of nucleotides 1-106 of SEQ ID NO:26 and a 3′ ITR comprising or consisting of nucleotides 2161-2305 of SEQ ID NO:26; a 5′ ITR comprising or consisting of nucleotides 1-106 of SEQ ID NO:27 and a 3′ ITR comprising or consisting of nucleotides 2161-2305 of SEQ ID NO:27; a 5′ ITR comprising or consisting of nucleotides 1-106 of SEQ ID NO:28 and a 3′ ITR comprising or consisting of nucleotides 2161-2305 of SEQ ID NO:28; a 5′ ITR comprising or consisting of nucleotides 1-106 of SEQ ID NO:29 and a 3′ ITR comprising or consisting of nucleotides 2266-2410 of SEQ ID NO:29; a 5′ ITR comprising or consisting of nucleotides 1-106 of SEQ ID NO:30 and a 3′ ITR comprising or consisting of nucleotides 2224-2368 of SEQ ID NO:30; a 5′ ITR comprising or consisting of nucleotides 1-106 of SEQ ID NO:31 and a 3′ ITR comprising or consisting of nucleotides 2216-2360 of SEQ ID NO:31; a 5′ ITR comprising or consisting of nucleotides 1-106 of SEQ ID NO:32 and a 3′ ITR comprising or consisting of nucleotides 2225-2369 of SEQ ID NO:32; a 5′ ITR comprising or consisting of nucleotides 1-106 of SEQ ID NO:33 and a 3′ ITR comprising or consisting of nucleotides 2266-2410 of SEQ ID NO:33; a 5′ ITR comprising or consisting of nucleotides 1-106 of SEQ ID NO:34 and a 3′ ITR comprising or consisting of nucleotides 2224-2368 of SEQ ID NO:34; a 5′ ITR comprising or consisting of nucleotides 1-106 of SEQ ID NO:35 and a 3′ ITR comprising or consisting of nucleotides 2216-2360 of SEQ ID NO:35; a 5′ ITR comprising or consisting of nucleotides 1-106 of SEQ ID NO:36 and a 3′ ITR comprising or consisting of nucleotides 2225-2369 of SEQ ID NO:36; a 5′ ITR comprising or consisting of nucleotides 1-106 of SEQ ID NO:37 and a 3′ ITR comprising or consisting of nucleotides 2257-2401 of SEQ ID NO:37; a 5′ ITR comprising or consisting of nucleotides 1-106 of SEQ ID NO:38 and a 3′ ITR comprising or consisting of nucleotides 2258-2402 of SEQ ID NO:38; a 5′ ITR comprising or consisting of nucleotides 1-106 of SEQ ID NO:39 and a 3′ ITR comprising or consisting of nucleotides 2215-2359 of SEQ ID NO:39; a 5′ ITR comprising or consisting of nucleotides 1-106 of SEQ ID NO:40 and a 3′ ITR comprising or consisting of nucleotides 2207-2351 of SEQ ID NO:40; a 5′ ITR comprising or consisting of nucleotides 1-106 of SEQ ID NO:41 and a 3′ ITR comprising or consisting of nucleotides 2207-2351 of SEQ ID NO:41; a 5′ ITR comprising or consisting of nucleotides 1-106 of SEQ ID NO:42 and a 3′ ITR comprising or consisting of nucleotides 2257-2401 of SEQ ID NO:42; a 5′ ITR comprising or consisting of nucleotides 1-106 of SEQ ID NO:43 and a 3′ ITR comprising or consisting of nucleotides 2215-2359 of SEQ ID NO:43; a 5′ ITR comprising or consisting of nucleotides 1-106 of SEQ ID NO:44 and a 3′ ITR comprising or consisting of nucleotides 2207-2351 of SEQ ID NO:44; a 5′ ITR comprising or consisting of nucleotides 1-106 of SEQ ID NO:78 and a 3′ ITR comprising or consisting of nucleotides 2258-2402 of SEQ ID NO:78; a 5′ ITR comprising or consisting of nucleotides 1-106 of SEQ ID NO:79 and a 3′ ITR comprising or consisting of nucleotides 2267-2411 of SEQ ID NO:79; a 5′ ITR comprising or consisting of nucleotides 1-106 of SEQ ID NO:80 and a 3′ ITR comprising or consisting of nucleotides 2251-2395 of SEQ ID NO:80; a 5′ ITR comprising or consisting of nucleotides 1-106 of SEQ ID NO:81 and a 3′ ITR comprising or consisting of nucleotides 2251-2395 of SEQ ID NO:81; or a 5′ ITR comprising or consisting of nucleotides 1-106 of SEQ ID NO:82 and a 3′ ITR comprising or consisting of nucleotides 2187-2331 of SEQ ID NO:82.

In some embodiments, the rAAV vector is a mammalian serotype AAV vector (e.g., AAV genome and ITRs derived from mammalian serotype AAV), including a primate serotype AAV vector or human serotype AAV vector. In some embodiments, the rAAV vector is a chimeric AAV vector. In some embodiments, the ITRs are selected from AAV serotypes of AAV1, AAV2, AAV3, AAV4, AAV5, AAV6, AAV7, AAV8, AAV9, AAV9.47, AAV9(hu14), AAV10, AAV11, AAV 12, AAVrh8, AAVrh10, AAV-DJ8, AAV-DJ, AAV-PUPA, AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A 15/G2A3, AAVG2B4, AAVG2B5, or variants thereof.

In some embodiments, the heterologous nucleic acid sequence encodes a therapeutic agent. In some embodiments, the therapeutic agent comprises a nucleic acid encoding a therapeutic protein or an inhibitory nucleic acid. In some embodiments, the inhibitory nucleic acid comprises a siRNA, miRNA, shRNA, or dsRNA. In some embodiments, the inhibitory nucleic acid comprises a siRNA, miRNA, shRNA, of dsRNA targeting a neurodegenerative disease related gene. In some embodiments, the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease. In some embodiments, the neurodegenerative disease is a polyglutamine repeat disease. In some embodiments, the inhibitory nucleic acid (e.g., siRNA, miRNA, shRNA, or dsRNA) targets ATXA2. In some embodiments, ATXA2 refers to mammalian ATNX2, including human ATXN2.

In some embodiments, the expression construct comprises a heterologous nucleic acid encoding an artificial miRNA targeting ATXN2. Examples of heterologous nucleic acids encoding guide sequences and passenger sequences and artificial miRNAs targeting ATXN2 are provided in Table 5. RNA formats of the encoded guide, passenger, and artificial miRNA sequences are provided, as well as the DNA formats for insertion into cis plasmids and rAAV vectors. In some embodiments, a heterologous nucleic acid encodes an artificial miRNA targeting ATXN2 and comprises a guide sequence selected from SEQ ID NOS:1-4 and 71. In some embodiments, a heterologous nucleic acid encodes an artificial miRNA comprising a guide sequence provided by SEQ ID NO:1 and a passenger sequence provided by SEQ ID NO:5. In some embodiments, a heterologous nucleic acid comprises a guide sequence provided by SEQ ID NO:2 and a passenger sequence provided by SEQ ID NO:6. In some embodiments, a heterologous nucleic acid comprises a guide sequence provided by SEQ ID NO:3 and a passenger sequence provided by SEQ ID NO:7. In some embodiments, a heterologous nucleic acid comprises a guide sequence provided by SEQ ID NO:4 and a passenger sequence provided by SEQ ID NO:8. In some embodiments, a heterologous nucleic acid comprises a guide sequence provided by SEQ ID NO:71 and a passenger sequence provided by SEQ ID NO:72. In some embodiments, the heterologous nucleic acid comprises a guide sequence and passenger sequence embedded in a miRNA backbone (or scaffold). In some embodiments, the miRNA backbone is miR-100 or miR-1-1. In some embodiments, the heterologous nucleic acid encodes an artificial miRNA sequence provided by any one of SEQ ID NOs:9-12 and 73. Additional examples of ATXN2 targeting guide sequences, passenger sequences, miR backbones, and artificial miRNA sequences are provided in PCT Publication WO2021/159008, which is incorporated by reference in its entirety.

Other expression control sequences may be present in the rAAV vector operably linked to the heterologous nucleic acid (e.g., encoding an inhibitory nucleic acid), including one or more of transcription initiation sequences, termination sequences, promoter sequences, enhancer sequences, repressor sequences, splice site sequences, polyadenylation (polyA) signal sequences, or any combination thereof.

In some embodiments, rAAV vector comprises a H1 promoter. In some embodiments, the H1 promoter is a native H1 promoter, such as a promoter comprising or consisting of the sequence set forth in SEQ ID NO:53. In some embodiments, the promoter is a short H1 promoter, such as a promoter comprising or consisting of the sequence set forth in SEQ ID NO:54 or nucleotides 113-203 of any one of SEQ ID NOS:17, 25-28, and 37-44. In some embodiments, the promoter is a long H1 promoter, such as a promoter comprising or consisting of the sequence set forth in SEQ ID NO:52 or nucleotides 113-343 of any one of SEQ ID NOS:13-16 and 18-24. In some embodiments, the H1 promoter is oriented in the 5′ to 3′ direction in the expression construct, particularly when the 5′ ITR lacks a terminal resolution site.

In some embodiments, the rAAV vector comprises a SV40 termination sequence. Examples of SV40 termination sequence are set forth in SEQ ID NO:77; nucleotides 489-710 of any one of SEQ ID NOS:20, 21, 23, and 24; nucleotides 358-579 of any one of SEQ ID NOS:31, 32, 35, and 36; nucleotides 349-570 of any one of SEQ ID NOS:40, 41, and 44.

rAAV vectors may have one or more AAV wild type genes deleted in whole or in part. In some embodiments the rAAV vector is replication defective. In some embodiments, the rAAV vector lacks a functional Rep protein and/or capsid protein. In some embodiments, the rAAV vector is a self-complementary AAV (scAAV) vector.

In some embodiments, the rAAV vector comprises: a 5′ ITR, a promoter operably linked to a heterologous nucleic acid encoding an ATXN2 specific artificial miRNA, a vector stuffer sequence, and a 3′ ITR. In some embodiments, the 5′ ITR is modified to lack a terminal resolution site. In some embodiments, the promoter is orientated in the 5′ to 3′ direction.

In some embodiments, the rAAV vector comprises from 5′ ITR to 3′ ITR the nucleotide sequence set forth in any one of SEQ ID NOS:13-24, 29-44, and 78-80. In some embodiments, the rAAV vector comprises from 5′ ITR to 3′ ITR the nucleotide sequence set forth in SEQ ID NO:13, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:14, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:15, wherein nucleotides 344-481 are substituted with a sequence encoding an artificial miRNA of interest. In some embodiments, the rAAV vector comprises the nucleotide sequence set forth in SEQ ID NO:16, wherein nucleotides 344-481 are substituted with a sequence encoding an artificial miRNA of interest. In some embodiments, the rAAV vector comprises the nucleotide sequence set forth in SEQ ID NO:17, wherein nucleotides 204-335 are substituted with a sequence encoding an artificial miRNA of interest. In some embodiments, the rAAV vector comprises the nucleotide sequence set forth in SEQ ID NO:18, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:19, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:20, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:21, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:22, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:23, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:24, wherein nucleotides 344-481 are substituted with a sequence encoding an artificial miRNA of interest. In some embodiments, the rAAV vector comprises from 5′ ITR to 3′ ITR the nucleotide sequence set forth in SEQ ID NO:29, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:30, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:31, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:32, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:33, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:34, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:35, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:36, wherein nucleotides 213-350 are substituted with a sequence encoding an artificial miRNA of interest. In some embodiments, the rAAV vector comprises from 5′ ITR to 3′ ITR the nucleotide sequence set forth in SEQ ID NO:37, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:38, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:39, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:40, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:41, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:42, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:43, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:44, wherein nucleotides 204-341 are substituted with a sequence encoding an artificial miRNA of interest. In some embodiments, the rAAV vector comprises from 5′ ITR to 3′ ITR the nucleotide sequence set forth in SEQ ID NO:78, wherein nucleotides 204-342 are substituted with a sequence encoding an artificial miRNA of interest. In some embodiments, the rAAV vector comprises from 5′ ITR to 3′ ITR the nucleotide sequence set forth in SEQ ID NO:79, wherein nucleotides 213-351 are substituted with a sequence encoding an artificial miRNA of interest. In some embodiments, the rAAV vector comprises from 5′ ITR to 3′ ITR the nucleotide sequence set forth in SEQ ID NO:80, wherein nucleotides 204-335 are substituted with a sequence encoding an artificial miRNA of interest. In some embodiments, the rAAV vector comprises from 5′ ITR to 3′ ITR the nucleotide sequence set forth in SEQ ID NO:81, wherein nucleotides 204-335 are substituted with a sequence encoding an artificial miRNA of interest.

Recombinant AAV vectors of the present disclosure may be encapsidated by one or more AAV capsid proteins to form a rAAV particle. A “rAAV particle” or “rAAV virion” refers to an infectious, replication-defective virus including an AAV protein shell, encapsidating a rAAV vector comprising a transgene of interest, which is flanked on each side by a 5′ AAV ITR and 3′ AAV ITR. A rAAV particle is produced in a suitable host cell which has had sequences specifying a rAAV vector, AAV helper functions and accessory functions introduced therein to render the host cell capable of encoding AAV polypeptides that are required for packaging the rAAV vector (containing the transgene sequence of interest) into infectious rAAV particles for subsequent gene delivery.

Methods of packaging recombinant AAV vector into AAV capsid proteins using host cell culture are known in the art. In some embodiments, one or more of the required components for packaging the rAAV vector, (e.g., Rep sequence, cap sequence, and/or accessory functions) may be provided by a stable host cell that has been engineered to to contain the one or more required components (e.g., by a vector). Expression of the required components for AAV packaging may be under control of an inducible or constitutive promoter in the host packaging cell. AAV helper vectors are commonly used to provide transient expression of AAV rep and/or cap genes, which function in trans, to complement missing AAV functions that are necessary for AAV replication. In some embodiments, AAV helper vectors lack AAV ITRs and can neither replicate nor package themselves. AAV helper vectors can be in the form of a plasmid, phage, transposon, cosmid, virus, or virion.

In some embodiments, rAAV particles may be produced using the triple transfection method (see, e.g., U.S. Pat. No. 6,001,650, incorporated herein by reference in its entirety). In this approach, the rAAV particles are produced by transfecting a host cell with a rAAV vector (comprising a transgene) to be packaged into rAAV particles, an AAV helper vector, and an accessory function vector. In some embodiments, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. In some embodiments, a double transfection method, wherein the AAV helper function and accessory function are cloned on a single vector, which is used to generate rAAV particles.

The AAV capsid is an important element in determining these tissue-specificity of the rAAV particle. Thus, a rAAV particle having a capsid tissue specificity can be selected. In some embodiments, the rAAV particle comprises a capsid protein selected from a AAV serotype of AAV1, AAV2, AAV3, AAV4, AAV5, AAV6, AAV7, AAV8, AAV9, AAV9.47, AAV9(hu14), AAV10, AAV11, AAV 12, AAVrh8, AAVrh10, AAV-DJ8, AAV-DJ, AAV-PUPA, AAV-PHP.B, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A 15/G2A3, AAVG2B4, AAVG2B5, and variants thereof. In some embodiments, the AAV capsid is selected from a serotype that is capable of crossing the blood-brain barrier, e.g., AAV9, AAVrh.10, AAV-PHP-B, or a variant thereof. Examples of AAV9 capsid sequences are provided in U.S. Pat. No. 7,906,111, incorporated by reference in its entirety. In some embodiments, the AAV capsid is a chimeric AAV capsid. In some embodiments, the AAV particle is a pseudotyped AAV, having capsid and genome from different AAV serotypes.

In some embodiments, the rAAV particle is capable of transducing cells of the CNS. In some embodiments, the rAAV particle is capable of transducing non-neuronal cells or neuronal cells of the CNS. In some embodiments, the CNS cell is a neuron, glial cell, astrocyte, or microglial cell.

In another aspect, the present disclosure provides host cells transfected with the rAAV particles comprising the vector stuffer sequences described herein. In some embodiments, the host cell is a prokaryotic cell or a eukaryotic cell. In some embodiments, the host cell is a mammalian cell (e.g., HEK293T, COS cells, HeLa cells, KB cells), bacterial cell (E. coli), yeast cell, insect cell (Sf9, Sf21, Drosophila, mosquito), etc.

### Methods of Use

In another aspect, the present disclosure provides methods of delivering a therapeutic agent to a subject, comprising administering a composition of the present disclosure (e.g., rAAV particle comprising a rAAV vector comprising the vector stuffer sequence provided herein and an expression construct comprising heterologous nucleic acid sequence encoding a therapeutic agent). In some embodiments, the cell is a CNS cell. In some embodiments, the cell is a non-neuronal cell or neuronal cell of the CNS. In some embodiments, the non-neuronal cell of the CNS is a glial cell, astrocyte, or microglial cell. In some embodiments, the cell is in vitro. In some embodiments, the cell is from a subject having one or more symptoms of a neurodegenerative disease or suspected of having a neurodegenerative disease.

As used herein, the term “treat” refers to preventing or delaying onset of neurodegenerative disease (e.g., ALS/FTD, Alzheimer's disease, Parkinson's disease, etc.); reducing severity of neurodegenerative disease; reducing or preventing development of symptoms characteristic of neurodegenerative disease; preventing worsening of symptoms characteristic of neurodegenerative disease, or any combination thereof.

In some embodiments, the subject has a neurodegenerative disease or is at risk of developing a neurodegenerative disease. Examples of neurodegenerative diseases include spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, and Alzheimer's disease.

In some embodiments, the methods for treatment of the present disclosure reduces, prevents, or slows development or progression of one or more symptom characteristic of a neurodegenerative disease. Examples of symptoms characteristic of neurodegenerative disease include motor dysfunction, cognitive dysfunction, emotional/behavioral dysfunction, or any combination thereof. Paralysis, shaking, unsteadiness, rigidity, twitching, muscle weakness, muscle cramping, muscle stiffness, muscle atrophy, difficulty swallowing, difficulty breathing, speech and language difficulties (e.g., slurred speech), slowness of movement, difficulty with walking, dementia, depression, anxiety, or any combination thereof.

In some embodiments, the methods for treatment of the present disclosure of the present disclosure comprise administration as a monotherapy or in combination with one or more additional therapies for the treatment of the neurodegenerative disease. Combination therapy may mean administration of the compositions of the present disclosure to the subject concurrently, prior to, subsequent to one or more additional therapies. Concurrent administration of combination therapy may mean that the the compositions of the present disclosure and additional therapy are formulated for administration in the same dosage form or administered in separate dosage forms.

In some embodiments, a subject treated in any of the methods described herein is a mammal (e.g., mouse, rat), preferably a primate (e.g., monkey, chimpanzee), or human.

In any of the methods described herein, a composition of the present disclosure may be administered to the subject by any route, including enteral (e.g., oral), parenteral, intravenous, intramuscular, intra-arterial, intramedullary, intrathecal, subpial, intraparenchymal, intrastriatal, intracranial, intracisternal, intra-cerebral, intracerebral ventricular, intraocular, intraventricular, intralumbar, subcutaneous, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors including the nature of the agent (e.g., its stability in the environment of the gastrointestinal tract), and/or the condition of the subject. In some embodiments, compositions are directly injected into the CNS of the subject. In some embodiments, compositions are injected by intrathecal, subpial, intraparenchymal, intrastriatal, intracranial, intracisternal, intra-cerebral, intracerebral ventricular, intraocular, intraventricular, intralumbar administration, or any combination thereof.

In some embodiments, a composition of the present disclosure is directly injected into the CNS of the subject. In some embodiments, direct injection into the CNS is intracerebral injection, intraparenchymal injection, intrathecal injection, intrastriatal injection, subpial injection, or any combination thereof. In some embodiments, direct injection into the CNS is direct injection into the cerebrospinal fluid (CSF) of the subject, optionally wherein the direct injection is intracisternal injection, intraventricular injection, intralumbar injection, or any combination thereof.

### EXAMPLES

### Example 1: Design of Vector “Stuffer” Sequence

### Selection of “Dispensable” Human Genomic DNA for Base Stuffer Sequence

In order to minimize any undesirable effect of the stuffer sequences, “dispensable” regions of the human genome were identified having two intended properties: (a) sequences with minimal impact if integration in the genome occurs (although this is thought to be very rare for AAV); and (b) sequences with minimal risk of initiating unexpected transcription. Therefore, regions of the genome in which i) deletions and duplications were common in the population and not associated with disease-relevant phenotypes (no evidence of evolutionary pressure) were examined; and/or ii) RNA expression across human tissues was low or undetectable (lack strong intrinsic enhancers/promoter elements). From this search, two regions were prioritized:

Region I: AMELY Gene Region (chrY: 6,865,918-6,874,027, HG38)

Multiple lines of evidence suggest the AMELY gene region is not under evolutionary selection. First, the AMELY region arose from an ancient duplication event and have functional homologs on chromosome X that are under selection. (Lahn, & Page, Science (1999) 286, 964-967). Second, the AMELY gene has more protein altering variants (including predicted loss of function variants) than expected with no evidence of recent positive selection (Nature (2020) 581, 434-443; https://gnomad.broadinstitute.org/gene/ENSG00000099721?dataset=gnomad_r2_1). Third, genetic variants of the AMELY region have been shown to have no phenotypic impact in humans for deletions or duplications. Duplication of AMELY is common and has no phenotype (Hum Genet. (2015) 134:789-800). Fourth, deletions are common and have no detectable phenotype (Hum Mol Genet (2007) 16:307-16). Furthermore, AMELY has not been associated with any rare human diseases (Online Mendelian Inheritance in Man, OMIM.org.)

AMELY mRNA is not detectably expressed in most human tissues (GTEX, The GTEx Consortium. (Science (2020) 369:1318-1330), and only present in chromosome Y carriers (males). Further, AMELX (the functional homolog of AMELY) is involved in tooth enamel during development (Front Physiol. (2017) 8:435).

Region H. PSG Gene Region (chr19:43,511,809-43,530,631, HG38)

Similarly, multiple lines of evidence suggest the Pregnancy-specific Glycoprotein (PSG) is not under clear evolutionary selection. First, the PSG cluster arose from segmental duplication (Dumont and Eichler PLoS One (2013) 8:e75949). Second, there is no evidence that the region is under evolutionary constraint with more predicted loss of function variants than expected (Nature (2020) 581:434-443; https://gnomad.broadinstitute.org/gene/ENSG00000243130?dataset=gnomad_r2_1). Third, the PSG cluster has not been associated with any rare human diseases (Online Mendelian Inheritance in Man, OMIM.org).

The PSG region is typically only highly expressed by placenta during pregnancy, with no/low expression in other tissues (GTEX, The GTEx Consortium. (Science (2020) 369: 1318-1330).

### Modification of Base Stuffer Sequence

DNA sequences from the AMELY region (chrY:6,865,918-6,874,027, HG38) and PSG gene region (chr19:43,511,809-43,530,631, HG38) were modified with the goal of making the sequence “inert”, or lacking any expressed regions, known or predicted regulatory elements and repetitive elements (including retroviral and transposable elements). These changes were enforced most stringently for stuffer sequence intended to be use in the region comprising the packaged vector genome.

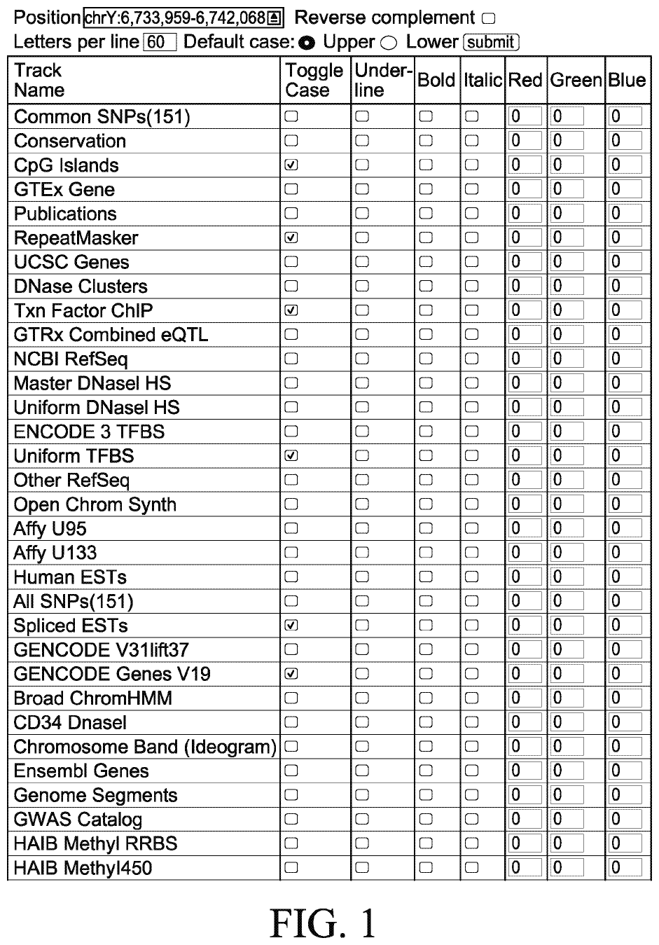

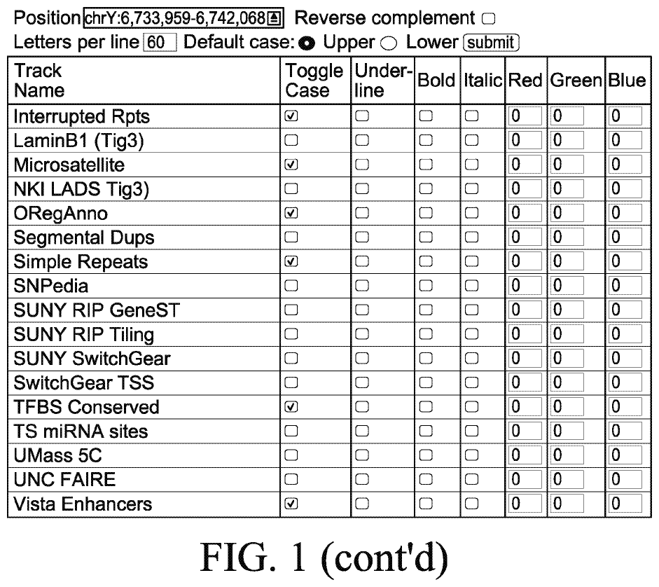

Specifically, the base sequences (chrY:6,865,918-6,874,027, HG38) and (chr19:43,511,809-43,530,631, HG38) were modified in the following manner (see FIG. 1):
i) Remove expressed regions: Exons+10 bp on either side (exons were defined using gene models in GENCODEv19), human expressed sequence tags (EST's: defined using “Human ESTs Including Unspliced” track on the UCSC genome browser, based off of Genbank data: (Nucleic Acids Res. (2004) 32(Database issue):D23-6.)
ii) Remove Regulatory Elements: The following tracks on the UCSC genome browser were used to define CpG Islands (J Mol Biol. (1987) 196(2):261-82), regions identified by CHiP seq (sequence bound by transcription factors) Transcription Factor ChIP-seq Peaks (340 factors in 129 cell types) from ENCODE 3 (Nature (2012) 489(7414):57-74.), conserved Transcription Factor Binding Sites (HMR Conserved Transcription Factor Binding Sites track), VISTA enhancers (Vista HMR-Conserved Non-coding Human Enhancers from LBNL track), and Open Regulatory Annotation “ORegAnno” (Regulatory elements from ORegAnno track; (Nucleic Acids Res. (2016) 44(D1):D126-32.).
iii) Remove repetitive elements: Repeat masked elements (including SINE,LINE, LTR, DNA elements) using the Repeating Elements by RepeatMasker track (http://www.repeatmasker.org), Microsatellite repeats defined by the Microsatellites—Di-nucleotide and Tri-nucleotide Repeats track (Nucleic Acids Res. (1999) 27(2):573-80, and interrupted and simple repeats defined by RepeatMasker (http://www.repeatmasker.org).
iv) To reduce the likelihood of unexpected translation, ATG residues were modified to reduce the frequency of ATG sequences.
v) Editing of CpG dinucleotides. To lower the likelihood of unmethylated CpG dinucleotides from cis plasmids generated in bacteria eliciting innate immune activation when packaged in AAV, the presence of CG dinucleotides were reduced by editing bases.

### Example 2: Evaluation of Safety of AAV Vector Genomes Packaged Using Stuffer Polynucleotides

Vectors incorporating stuffer sequences described according to Example 1, without inclusion of any additional active elements (such as miRNAs), were tested for safety in vivo. A set of 4 vectors were designed, with two variants each of AMELY and PSG11 derived-stuffers (SEQ ID NOS:25-28). Each vector contained a H1 short promoter at the 5′ side immediately downstream of the left ITR (SEQ ID NO:57), and a non-miRNA control sequence was included instead of a miRNA downstream of the H1 short promoter. A Pol III terminator is positioned 3′ of the control sequence. In parallel, three controls were tested: vehicle; a vector encoding an artificial miRNA sequence targeting ATXN2 (scAAV_H1_miR16-2-1479_AMELY_V1 (SEQ ID NO:81)) that was previously shown to exhibit toxicity when tested in lentiviral format on HeLa and U2OS cells, under the control of the H1 promoter; and a vector encoding an Atxn2 specific miRNA as well as GFP (scAAV_H1_miR1-1-XD-14792_CBh_GFP_SV40p (SEQ ID NO:82)). The artificial miRNA which was shown previously to exhibit toxicity was chosen to serve as a positive control for toxicity, presumably through an off-target effect of the specific artificial miRNA sequence.

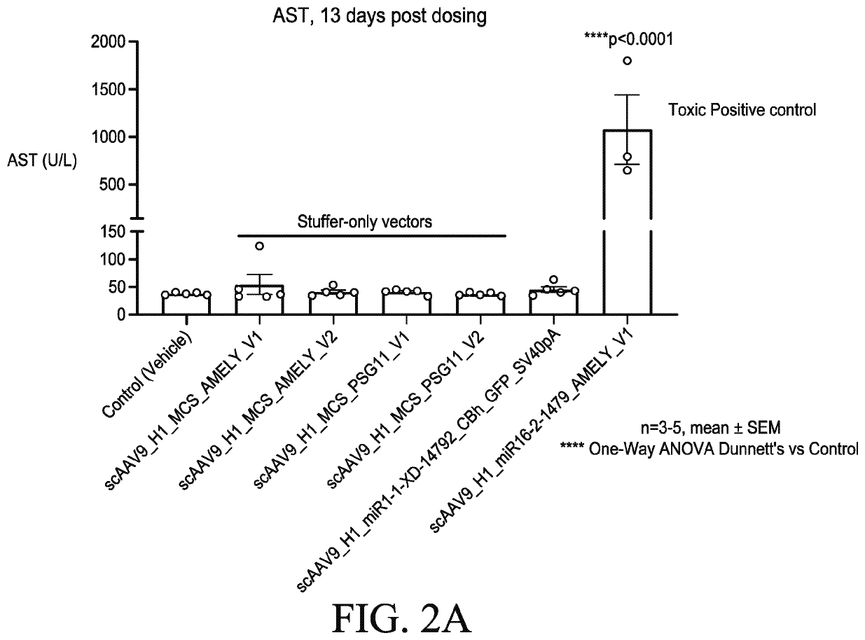

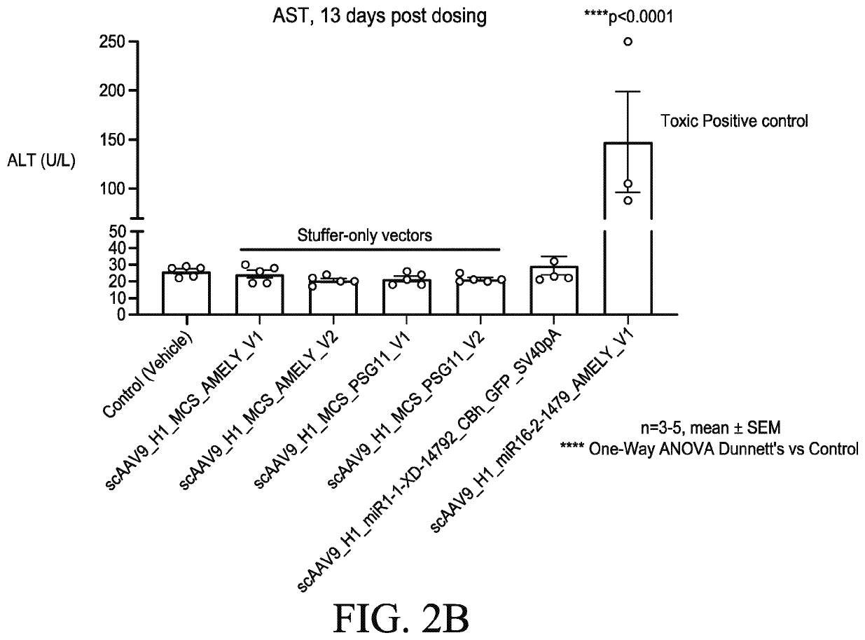

Vectors were dosed intravenously at 1.3E11 total viral genome (vg) via tail vein to 16 weeks of age wild-type C57Bl/6 mice. Titer was determined based on DNA concentration of column-purified vector. This concentration was compared to the concentration of a similarly purified vector that was also titered by vector genome qPCR, to calculate corresponding titers for the stuffer vectors described in this example. Blood was processed, and serum evaluated for the concentration of liver enzymes alanine transaminase (ALT) and aspartate transaminase (AST), as a proxy for any liver toxicity. The vector expressing the toxic positive control artificial miRNA elicited a substantial rise in liver enzymes ALT and AST. By contrast, none of the vectors with the stuffer sequences elicited a rise in liver enzymes (FIGS. 2A-2B).

A histological evaluation of liver was performed by a pathologist to evaluate any toxicity. 28 days after dosing, animals (n=5 animals per stuffer vector treatment) were sacrificed and livers immersion-fixed in 4% paraformaldehyde for 24 hours. Two cross sections per liver were embedded in paraffin, sectioned at 5 micron thickness, and stained with hematoxylin and eosin. Photomicrographs were taken on an Olympus BX60 microscope. Animals dosed with the toxic positive control miRNA exhibited a number of lesions including oval cell hyperplasia, hepatocellular fusion and multinucleation, lymphohistiocytic hepatitis, sinusoidal fibrosis, intrahistiocytic pigment, and rare single cell apoptosis/necrosis. In the stuffer containing vectors, histologic findings were limited to incidental background lesions that did not follow a treatment-related trend and were seen across multiple treatment groups. Findings included variation in hepatic glycogenosis (hepatocellular glycogen accumulation) and rare microgranulomas. The findings were not deemed to reflect compound-related treatment toxicity.

### Example 3: Evaluation of Vector Genome Truncations in Specific Stuffer Designs

rAAV vector genome preparations are known to contain truncated vector genomes, most pronounced at hairpin structures such as miRNAs but also at other regions of DNA secondary structure. Therefore, the presence of truncated genomes in preparations of rAAVs containing different forms of stuffer sequence was assessed.

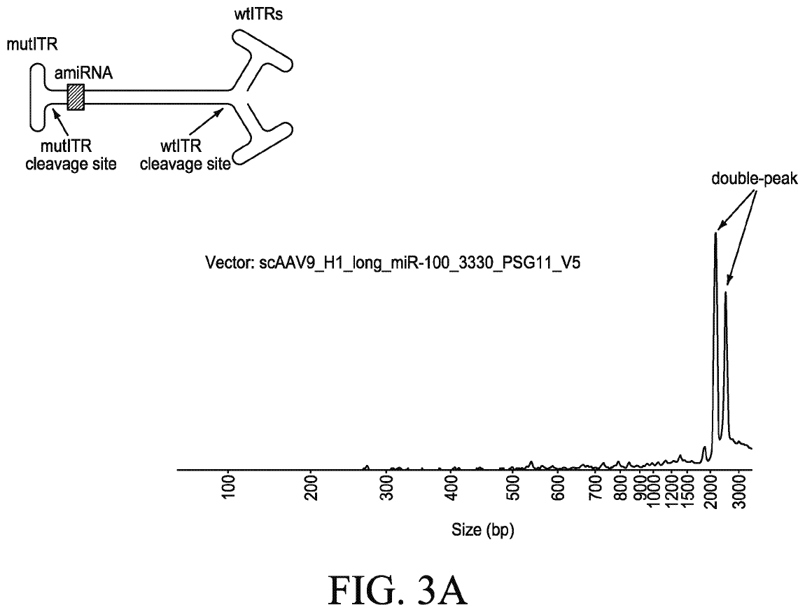

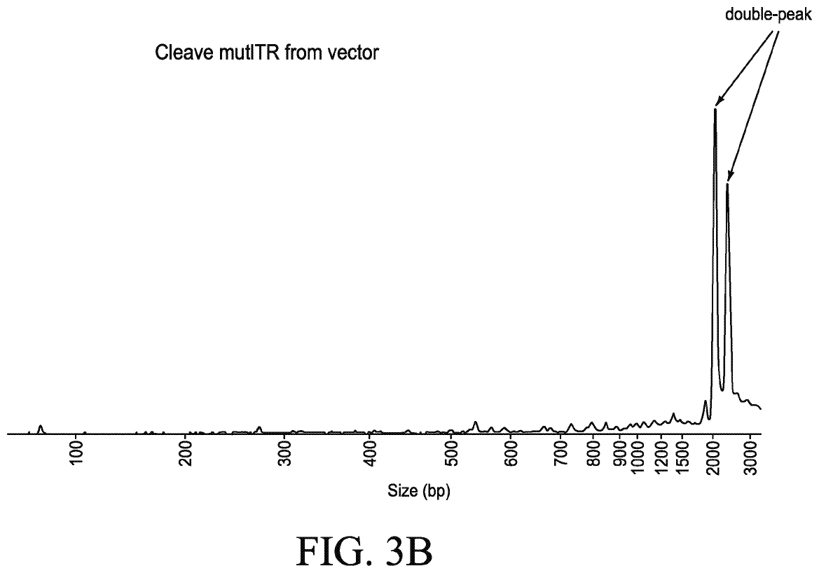

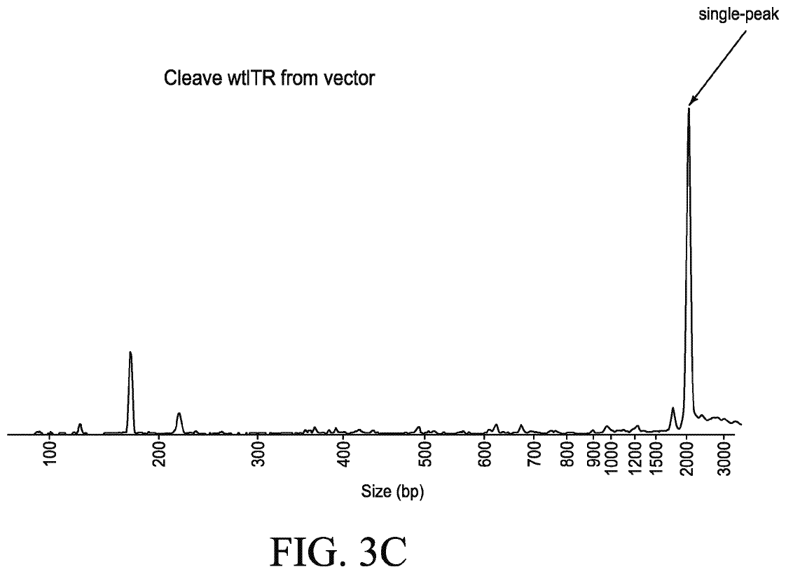

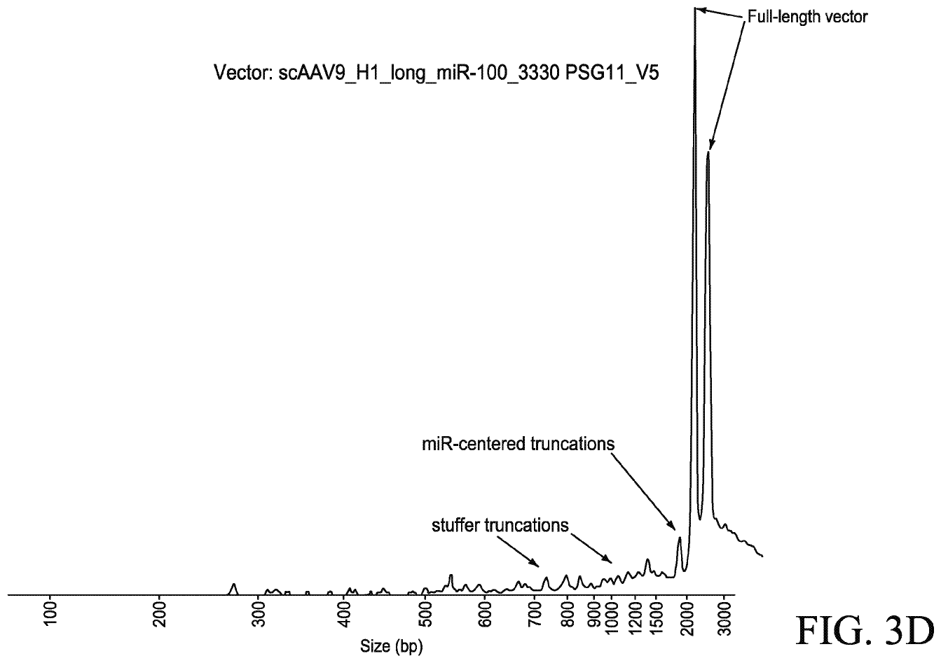

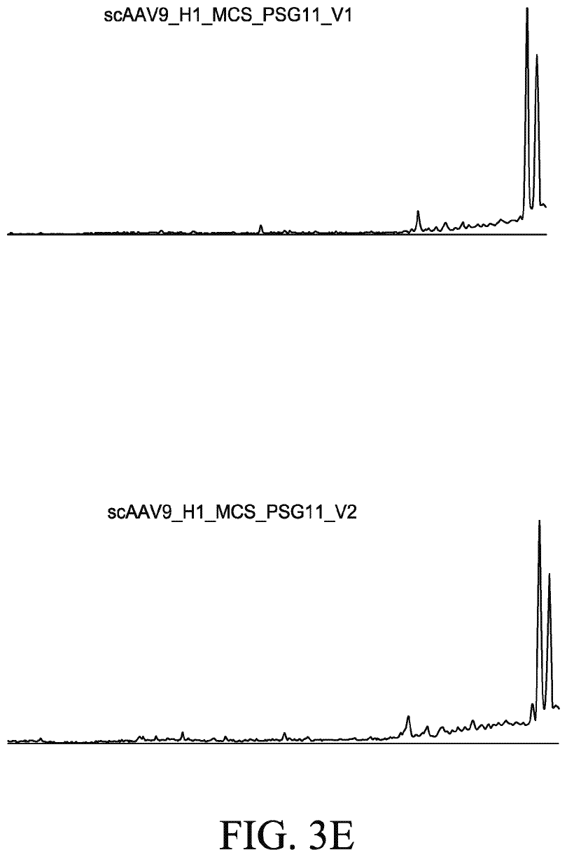

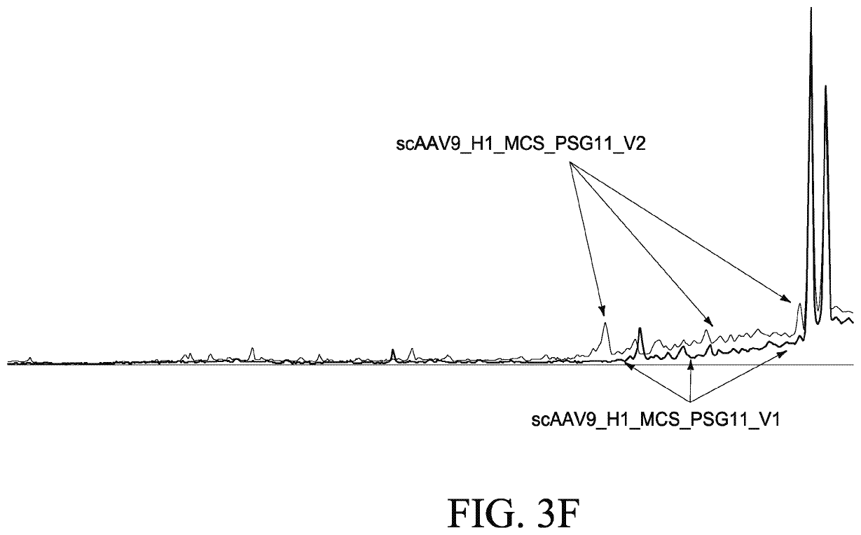

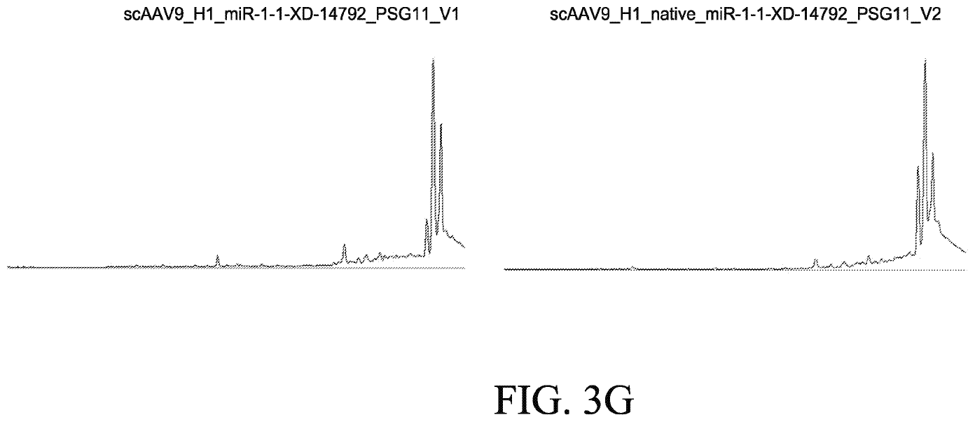

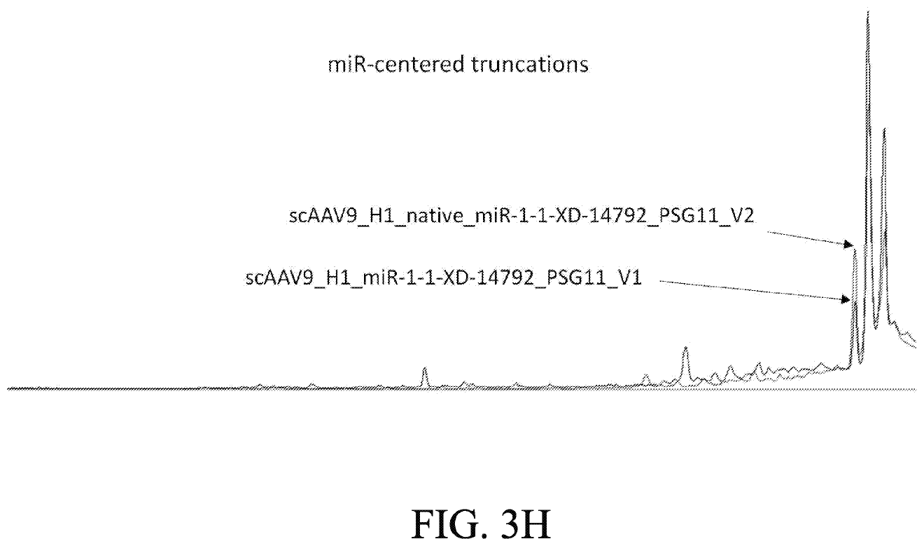

FIGS. 3A-3C show DNA traces from Fragment Analyzer analysis. Following rAAV vector (scAAV9_H1_long_miR-100_3330_PSG11_V5 (SEQ ID NO:16)) production, vector DNA was extracted and column purified (Zymo, P/N D3015) and run on Agilent Fragment Analyzer (HS NGS Fragment 1-6000 bp). A double-peaked trace can be seen (FIG. 3A), and these double peaks were determined to be full-length structures containing ITRs because they are resolved by restriction digestion at a site that removes the right-side wild-type ITR. A smaller shoulder to the left of the intended full-length product, indicative of shorter, truncated genomes, is also present. A sub-peak centered at the amiRNA, representative of vector genomes truncated at the amiRNA, is found in many preparations (FIG. 3D). A similar peak is present if a control sequence is used in the place of the amiRNA but containing secondary structure such as palindromic sequences.

FIGS. 3E-3F compare two rAAV preparations (scAAV9_H1_MCS_PSG11_V1 (SEQ ID NO:27) and scAAV9_H1_MCS_PSG11_V2 (SEQ ID NO:28)) derived from cis plasmids containing one of two versions of stuffer sequence derived from PSG11 (PSG11_V1 (SEQ ID NO:45) and PSG11_V2 (SEQ ID NO:46)). These sequences were structurally similar, though ATG sites were edited to eliminate ATG sequences in PSG11_V2. There were some structural differences in the 5′ part of the stuffer sequences, which abut the expression cassette (the H1 promoter and either amiRNA or, in the vectors shown in FIG. 3E, the control sequence). The traces show that the ‘shoulder’ to the left of the full-length product is smaller in the PSG11_V1-containing vector genome preparation (FIG. 3F). In these vector preparations, no amiRNA was used.

FIGS. 3G-3H compare a similar set of two rAAV preparations (scAAV9_H1_miR-1-1-XD-14792_PSG11_V1 (SEQ ID NO:78) and scAAV9_H1_native_miR-1-1-XD-14792_PSG11_V2 (SEQ ID NO:79)) derived from cis plasmids containing either of stuffer sequence PSG11_V1 (SEQ ID NO:45) or PSG11_V2 (SEQ ID NO:46). In this case, both vectors contain an amiRNA element downstream of an H1 promoter (there are 3 nucleotides inserted in the promoter sequence on the PSG11_V2-containing vector versus the V1 containing vector, but otherwise the 5′ sequences are identical). By inspection of the fragment analyzer trace (FIG. 3H), it can be seen that the truncation at the amiRNA site is considerably larger in the vector containing the PSG11_V2 stuffer sequence.

The replication of self-complementary AAV (scAAV) vectors such as those described here involves initiation of replication from the wild-type ITR. In the sequences described here, this is at the 3′ side in the cis plasmids. Thus, a polymerase will initially traverse going through the stuffer sequence from 3′ to 5′ toward the amiRNA. A potential explanation for the increased truncated products is that the secondary structure that the polymerase may encounter just prior to the hairpin of the amiRNA promotes strand-switching activity leading to exacerbated truncations.

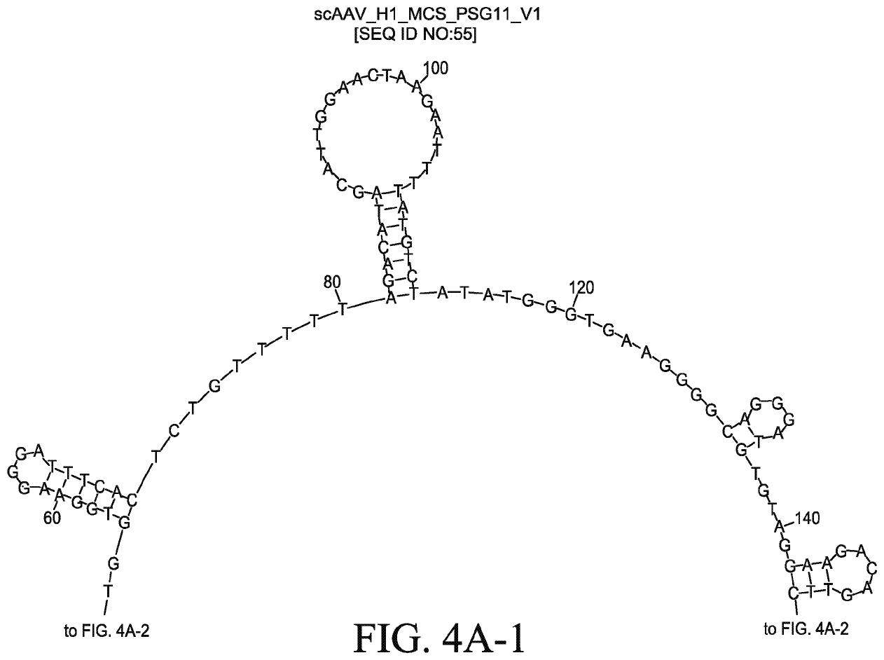

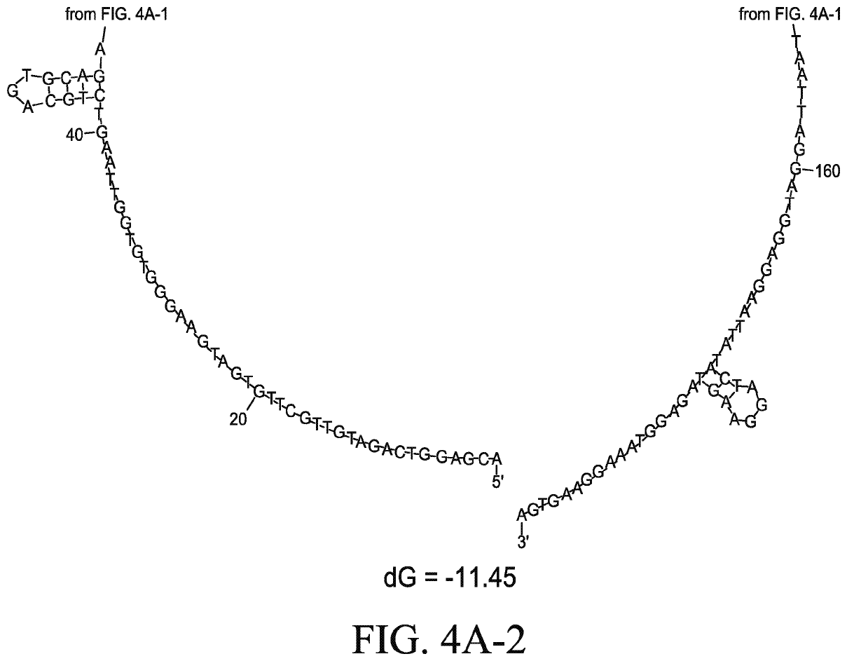

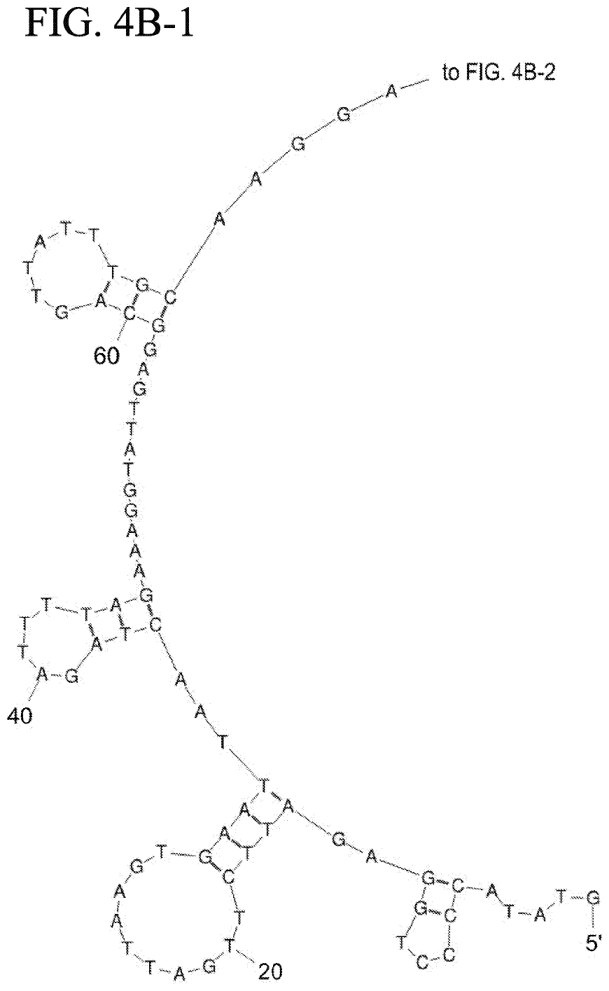

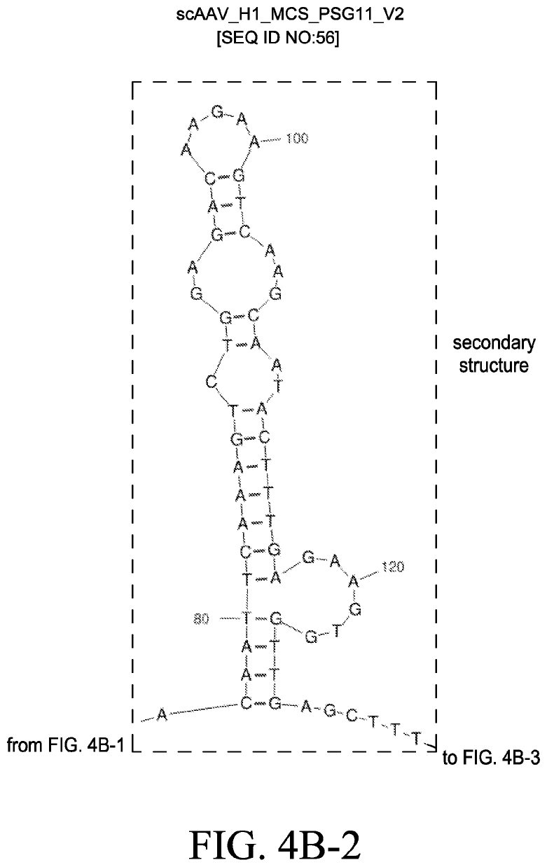

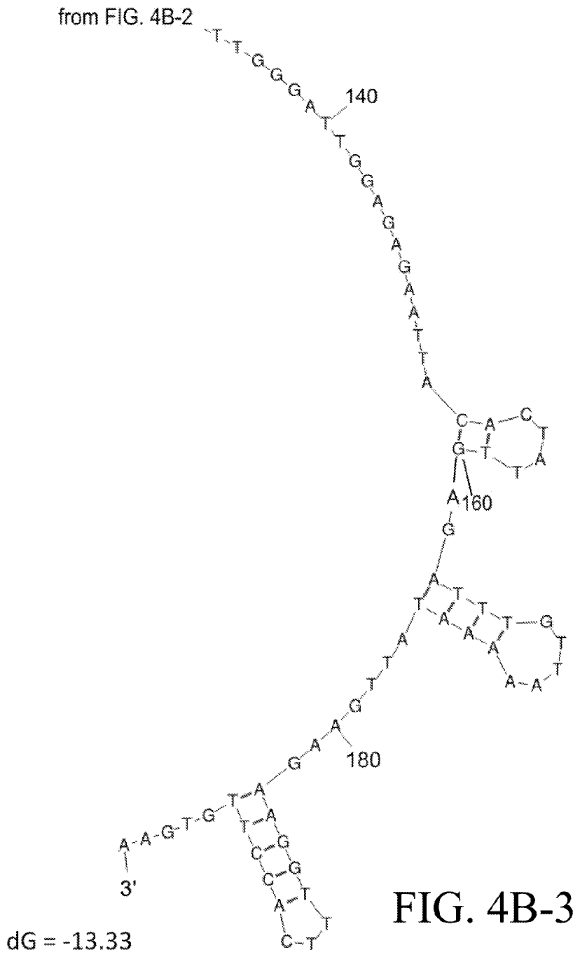

Consistent with this hypothesis, an examination of the folding of the 200 nucleotides of DNA sequence within the PSG11_V1 stuffer sequence (SEQ ID NO:55; FIG. 4A) versus PSG11_V2 stuffer sequence(SEQ ID NO:56; FIG. 4B) immediately 3′ of the amiRNA revealed a region of increased predicted secondary structure in the PSG11_V2 stuffers. Therefore, the PSG11_V1 sequence structure was favored. A “PSG11_V5” stuffer sequence (SEQ ID NO:48)) was generated by using the favorable sequence structure from the PSG11_V1 sequence. This is envisioned to yield vector preparations with reduced truncated vector genomes. The _V5 stuffer incorporated edits to CG sequences and ATG sequences to reduce the potential for hypomethylated CpG dinucleotides or unintended open reading frames.

### Example 4: Further Evaluation of Safety of Stuffer Sequences when Combined with ATXN2 AmiRNA Expression Cassettes

An experiment was conducted in which 24 different vector designs encompassing various stuffer sequences were evaluated for safety in vivo. Three different variants of the H1 promoter (H1 short (SEQ ID NO:54), H1 native (SEQ ID NO:53), H1 long (SEQ ID NO:52)), two different ATXN2 targeting artificial miRNA (3330 or 14792 (also referred to herein as 1784))/miR backbone (miR1.1. or miR100) combinations (miR100_3330 (encoding SEQ ID NO:12); miR1.1_14792 (encoding SEQ ID NO:73)), the presence or absence of a Pol-II transcriptional terminator SV40 polyadenylation sequence, and different versions of the stuffer sequences (AMELY_V3 (SEQ ID NO:51), PSG11_V2 (SEQ ID NO:46), PSG11_V3 (SEQ ID NO:47), PSG11_V5 (SEQ ID NO:48)) were assembled in various combinations (see, Table 1). As in the previous examples, in these vectors the H1 promoter is oriented from 5′ to 3′ in the cis plasmid intended for packaging as a self-complementary AAV vector, where the mutant ITR (lacking a terminal resolution site) is on the 5′ side (e.g., SEQ ID NO:57 or nucleotides 1-106 of any one of SEQ ID NOS:13-24, 29-44, and 78-82) and a wild-type AAV ITR is on the 3′ side (e.g., SEQ ID NO:58 or nucleotides 2214-2358 of any one of SEQ ID NOS:13-16). The reason for the use of this promoter orientation is that miR-centered truncations are possible in vectors incorporating ITRs lacking terminal resolution sites (self-complementary AAV vectors). In the 5′ to 3′ promoter orientation, vectors with miR-centered truncations will not include the promoter and therefore should be inert. If the promoter was in the 3′ to 5′ orientation, the promoter may be retained in a vector with a miR-centered truncation.

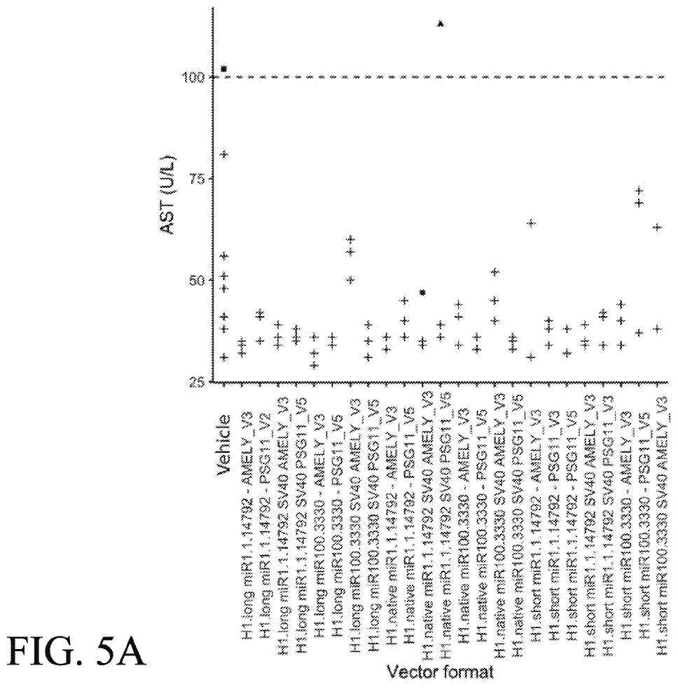

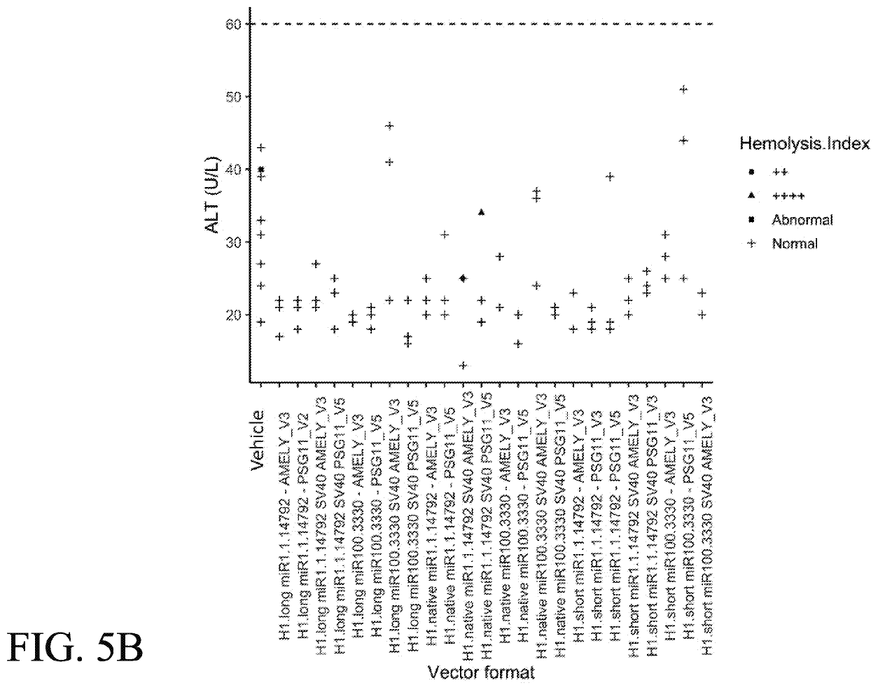

rAAV vector was generated with these cis plasmids and vectors were administered i.v. by tailvein injection, at a dose of 3.21E9 vector genomes per gram mouse, adjusted by weight (average total dose 8.5E10 vector genomes) to 12 weeks of age wild-type C57Bl/6 mice. Vehicle control animals were dosed with PBS with 0.001% PF-68. Blood was collected 2 weeks after administration by submandibular bleed, and liver enzyme concentrations (aspartate transaminase (AST) and alanine transaminase (ALT)) (FIGS. 5A-5B) were measured in animals dosed with the various vector designs. Almost all data points for animals treated with stuffer-containing vectors were within normal reference ranges. Although two datapoints show AST signal elevated above reference, hemolysis was noted in these samples and one sample was from a vehicle treated animal. In none of the other samples from animals dosed with any of the vector formats at this timepoint did AST or ALT rise above the reference range, suggesting there was not evidence of acute liver injury in response to rAAV dosing with vectors encoding these stuffer sequences.

TABLE 1
Summary of animals per vector dosed i.v. evaluated (liver histology)

| Vector | Promoter | miR | Terminator | Stuffer | N |
|---|---|---|---|---|---|
| scAAV_H1_short_miR100_3330_SV40_AMELY_V3 [SEQ ID NO: 44] | H1.short | miR100 - 3330 | SV40 | AMELY_V3 | 1 |
| scAAV_H1_short_miR100_3330_AMELY_V3 [SEQ ID NO: 42] | H1.short | miR100 - 3330 | None | AMELY_V3 | 1 |
| scAAV_H1_short_miR1-1_1784_SV40_AMELY_V3 [SEQ ID NO: 40] | H1.short | miR1.1 - 1784 | SV40 | AMELY_V3 | 1 |
| scAAV_H1_short_miR1-1_1784_AMELY_V3 [SEQ ID NO: 37] | H1.short | miR1.1 - 1784 | None | AMELY_V3 | 1 |
| scAAV_H1_native_miR100_3330_SV40_AMELY_V3 [SEQ ID NO: 35] | H1.native | miR100.3330 | SV40 | AMELY_V3 | 1 |
| scAAV_H1_native_miR100_3330_AMELY_V3 [SEQ ID NO: 33] | H1.native | miR100.3330 | None | AMELY_V3 | 1 |
| scAAV_H1_native_miR1-1_1784_SV40_AMELY_V3 [SEQ ID NO: 31] | H1.native | miR1.1.1784 | SV40 | AMELY_V3 | 2 |
| scAAV_H1_native_miR1-1_1784_AMELY_V3 [SEQ ID NO: 29] | H1.native | miR1.1.1784 | None | AMELY_V3 | 1 |
| scAAV_H1_long_miR100_3330_SV40_AMELY_V3 [SEQ ID NO: 23] | H1.long | miR100.3330 | SV40 | AMELY_V3 | 1 |
| scAAV_H1_long_miR100_3330_AMELY_V3 [SEQ ID NO: 22] | H1.long | miR100.3330 | None | AMELY_V3 | 1 |
| scAAV_H1_long_miR1-1_1784_SV40_AMELY_V3 [SEQ ID NO: 20] | H1.long | miR1.1.1784 | SV40 | AMELY_V3 | 1 |
| scAAV_H1_long_miR1-1_1784_AMELY_V3 [SEQ ID NO: 18] | H1.long | miR1.1.1784 | None | AMELY_V3 | 2 |
| scAAV_H1_short_miR1-1_1784_SV40_PSG11_V3 [SEQ ID NO: 41] | H1.short | miR1.1.1784 | SV40 | PSG11_V3 | 1 |
| scAAV_H1_short_miR1-1_1784_PSG11_V3 [SEQ ID NO: 38] | H1.short | miR1.1.1784 | None | PSG11_V3 | 1 |
| scAAV_H1_long_miR1-1_1784_PSG11_V2 [SEQ ID NO: 19] | H1.long | miR1.1.1784 | None | PSG11_V2 | 1 |
| scAAV_H1_short_miR100_3330_PSG11_V5 [SEQ ID NO: 43] | H1.short | miR100.3330 | None | PSG11_V5 | 1 |
| scAAV_H1_short_miR1-1_1784_PSG11_V5 [SEQ ID NO: 39] | H1.short | miR1.1.1784 | None | PSG11_V5 | 1 |
| scAAV_H1_native_miR100_3330_SV40_PSG11_V5 [SEQ ID NO: 36] | H1.native | miR100.3330 | SV40 | PSG11_V5 | 1 |
| scAAV_H1_long_miR1-1_1784_SV40_PSG11_V5 [SEQ ID NO: 21] | H1 long | miR1.1.1784 | SV40 | PSG11_V5 | 1 |
| scAAV_H1_native_miR100_3330_PSG11_V5 [SEQ ID NO: 34] | H1.native | miR100.3330 | None | PSG11_V5 | 1 |
| scAAV_H1_native_miR1-1_1784_SV40_PSG11_V5 [SEQ ID NO: 32] | H1.native | miR1.1.1784 | SV40 | PSG11_V5 | 1 |
| scAAV_H1_native_miR1-1_1784_PSG11_V5 [SEQ ID NO: 30] | H1.native | miR1.1.1784 | None | PSG11_V5 | 1 |
| scAAV_H1_long_miR100_3330_SV40_PSG11_V5 [SEQ ID NO: 24] | H1.long | miR100.3330 | SV40 | PSG11_V5 | 1 |
| scAAV_H1_long_miR100_3330_PSG11_V5 [SEQ ID NO: 16] | H1.long | miR100.3330 | None | PSG11_V5 | 1 |

Animals dosed intravenously with stuffer containing vectors were also evaluated for liver toxicity by histology. Three weeks after dosing, two consecutive 0.5 cm left liver lobe sections were collected per animal, embedded in paraffin, sectioned to 5 microns, and stained with hematoxylin and eosin. Slides were evaluated by a trained pathologist. N=1-2 animals per group were assessed, as shown in Table 1. After evaluation, intravenous administration of the test articles was not interpreted to be associated with changes in the examined sections of liver, for all vectors assessed.

### Intrastriatal Testing

TABLE 2
Summary of animals per vector dosed intrastriatally evaluated (striatum histology)

| | | | Pol II | | |
|---|---|---|---|---|---|
| Vector | Promoter | miR | Terminator | Stuffer | N |
| | N/A (Naïve) | | | | 2 |
| | N/A (Sham | | | | 2 |
| | injection, needle | | | | |
| | only) | | | | |
| | N/A (Vehicle, PBS + | | | | 2 |
| | .001% PF-68) | | | | |
| SEQ ID NO: 37 | H1.short | miR1.1.1784 | None | AMELY_V3 | 1 |
| SEQ ID NO: 29 | H1.native | miR1.1.1784 | None | AMELY_V3 | 1 |
| SEQ ID NO: 20 | H1.long | miR100.3330 | SV40 | AMELY_V3 | 1 |
| SEQ ID NO: 22 | H1.long | miR100.3330 | None | AMELY_V3 | 1 |
| SEQ ID NO: 18 | H1.long | miR1.1.1784 | None | AMELY_V3 | 2 |
| SEQ ID NO: 19 | H1.long | miR1.1.1784 | None | PSG11_V2 | 1 |
| SEQ ID NO: 43 | H1.short | miR100.3330 | None | PSG11_V5 | 2 |
| SEQ ID NO: 36 | H1.native | miR100.3330 | SV40 | PSG11_V5 | 1 |
| SEQ ID NO: 34 | H1.native | miR100.3330 | None | PSG11_V5 | 1 |
| SEQ ID NO: 24 | H1.long | miR100.3330 | SV40 | PSG11_V5 | 2 |
| SEQ ID NO: 16 | H1.long | miR100.3330 | None | PSG11_V5 | 2 |

These vectors were also dosed via direct injection into the striatum, at a dose of 7.5E9 vector genomes per striata to 8 week age wild-type C57Bl/6 mice, and a subset of vectors (Table 2, above) evaluated for toxicity by histological observation. Sections of striata from animals 3 weeks post-dosing were paraffin embedded, with four serial sections cut with a 5 micron block advance mounted to separate slides. A set of 2 slides containing step sections from three levels was included. Sections were stained with hematoxylin and eosin, glial fibrillary acid protein (GFAP) immunohistochemistry, ionized calcium binding adaptor molecule 1 (IBA-1) immunohistochemistry, and floro-jade B. The latter three stains can be used to evaluate astrocytic reactivity, microglial reactivity, and neuronal death, respectively.

As with the liver sections from animals treated with i.v. administered vector, there were no test article effects identified in the examined sections of brain. Handling artifacts limited interpretation in some tissues. In general, changes were observed at the injection site and included slight disruption of the tissue with light gliosis (identified on H&E-stained slides), slight microgliosis (identified on GFAP-labeled slides), and subtle hemosiderin pigment. The hemosiderin pigment is a common change with intraparenchymal injections. Test articles were not interpreted to cause an exacerbation or changes associated with the experimental procedures in any of the examined sections at the level of the basal nuclei/striatum from any animal.

### Transcriptional Analysis

To further evaluate the safety of the vectors, sequencing analysis was performed on striatal punch biopsies from animals dosed with the above vectors. Striatal tissue was column purified to extract RNA (after DNase-treatment) using Qiagen's AllPrep DNA/RNA/Protein Mini Kit (Qiagen, P/N 80004). Stranded RNA-seq libraries were prepared with the Stranded Total RNA Prep Ligation with Ribo-Zero Plus kit (Illumina, P/N 20040525) followed by paired end 2×100 bp sequencing on the Illumina Novaseq 6000 system. These reads were then aligned to the respective cis plasmid used to package the rAAV administered to that animal. Separately, DNA was purified from the same punch biopsy that had been used to extract the sequenced RNA (Qiagen, P/N 80004), and the vector exposure confirmed. Table 3 lists the exposures for each sample tested, along with the vector.

TABLE 3
Vector biodistribution in samples assessed by stranded RNA-seq

| Exposure | |
|---|---|
| (VG/diploid | |
| genome) | Construct |
| 142 | scAAV_H1_long_miR100_3330_AMELY_V3 [SEQ ID NO: 22] |
| 101 | scAAV_H1_long_miR100_3330_AMELY_V3 [SEQ ID NO: 22] |
| 53 | scAAV_H1_long_miR1-1_1784_AMELY_V3 [SEQ ID NO: 18] |
| 85 | scAAV_H1_long_miR1-1_1784_AMELY_V3 [SEQ ID NO: 18] |
| 63 | scAAV_H1_long_miR100_3330_PSG11_V5 [SEQ ID NO: 16] |
| 71 | scAAV_H1_long_miR100_3330_PSG11_V5 [SEQ ID NO: 16] |
| 56 | scAAV_H1_short_miR100_3330_AMELY_V3 [SEQ ID NO: 42] |
| 80 | scAAV_H1_short_miR100_3330_AMELY_V3 [SEQ ID NO: 42] |
| 82 | scAAV_H1_short_miR1-1_1784_SV40_AMELY_V3 [SEQ ID NO: 40] |
| 60 | scAAV_H1_short_miR1-1_1784_SV40_AMELY_V3 [SEQ ID NO: 40] |
| 77 | scAAV_H1_long_miR100_3330_SV40_PSG11_V5 [SEQ ID NO: 24] |
| 65 | scAAV_H1_long_miR100_3330_SV40_PSG11_V5 [SEQ ID NO: 24] |
| 67 | scAAV_H1_short_miR1-1_1784_AMELY_V3 [SEQ ID NO: 37] |
| 52 | scAAV_H1_short_miR1-1_1784_AMELY_V3 [SEQ ID NO: 37] |
| 75 | scAAV_H1_short_miR1-1_1784_PSG11_V3 [SEQ ID NO: 38] |
| 106 | scAAV_H1_short_miR1-1_1784_PSG11_V3 [SEQ ID NO: 38] |
| 121 | scAAV_H1_short_miR1-1_1784_PSG11_V5 [SEQ ID NO: 39] |
| 28.9 | scAAV_H1_short_miR1-1_1784_PSG11_V5 [SEQ ID NO: 39] |
| 136 | scAAV_H1_long_miR1-1_1784_PSG11_V2 [SEQ ID NO: 19] |
| 36.6 | scAAV_H1_long_miR100_3330_SV40_AMELY_V3 [SEQ ID NO: 23] |
| 55 | scAAV_H1_short_miR1-1_1784_SV40_PSG11_V3 [SEQ ID NO: 41] |
| 70 | scAAV_H1_short_miR1-1_1784_SV40_PSG11_V3 [SEQ ID NO: 41] |
| 37.7 | scAAV_H1_long_miR100_3330_SV40_AMELY_V3 [SEQ ID NO: 23] |
| 147 | scAAV_H1_long_miR1-1_1784_PSG11_V2 [SEQ ID NO: 19] |

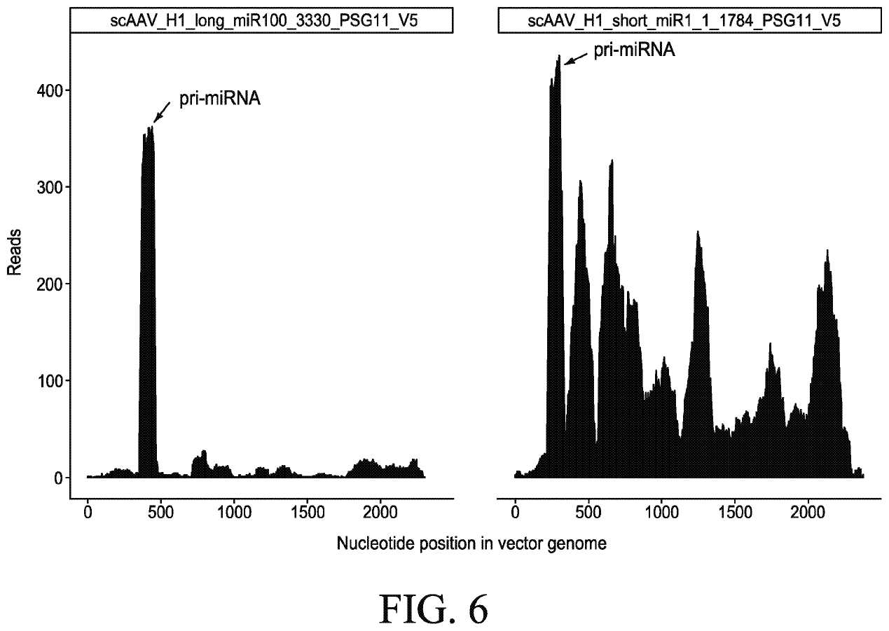

FIG. 6 shows pileup of reads from representative vectors. Surprisingly, the use of the H1 short promoter and miR 1.1 backbone along with the PSG11 V5 stuffer sequence led to an increased number of reads aligning to the cis plasmid downstream of the expected transcription of the pri-miRNA. By contrast, the use of the H1 long promoter with the PSG11 V5 stuffer led to a beneficial minimal amount of unintended transcription. Thus, this combination of elements produced a vector architecture with the desired low frequency of non-miRNA transcription.

### Example 5: Demonstration of Functionality of Stuffer Sequences when Combined with ATXN2 AmiRNA Expression Cassettes in Different RAAV Vector Formats

Animals dosed with the set of 24 vector combinations described in Example 4 were also assessed for functionality of all of the combinations of stuffer, promoter, and ATXN2-targeting artificial miRNAs. Knockdown of Atxn2 was assessed by extracting RNA from liver tissue 3 weeks after i.v. dosing (dose: 3.21E9 vg/gram mouse), then performing RT-ddPCR with Atxn2 and the control probe Hprt. Atxn2 levels were calculated as the mean of these ATXN2/control transcript ratios, and further normalized to the ratios obtained for animals dosed with vehicle. Vector genome exposures were also measured, using ddPCR with probes against the vector genome and a mouse genome probe (Tert). As seen in Table 4, robust Atxn2 knockdown occurred in animals treated with all combinations of stuffer sequences and miRNA vector components.

TABLE 4
Liver Atxn2 knockdown by various vectors containing stuffer sequences

| Promoter | miR | Terminator | Stuffer | mean_ratio | SD_ratio | count | mean_VG | SD_VG |
|---|---|---|---|---|---|---|---|---|
| N/A (Vehicle) | | z | | 100 | 16.54 | 18 | 0.01 | 0.01 |
| H1.long | miR1.1.14792 | None | AMELY_V3 | 8.68 | 2.88 | 3 | 6.65 | 1.07 |
| H1.long | miR1.1.14792 | None | PSG11_V2 | 6.07 | 0.99 | 3 | 65 | 14.73 |
| H1.long | miR1.1.14792 | SV40 | AMELY_V3 | 13.37 | 4.52 | 3 | 5.36 | 1.47 |
| H1.long | miR1.1.14792 | SV40 | PSG11_V5 | 9.25 | 4.52 | 3 | 9.32 | 0.98 |
| H1.long | miR100.3330 | None | AMELY_V3 | 15.99 | 1.91 | 3 | 8.93 | 2.07 |
| H1.long | miR100.3330 | None | PSG11_V5 | 13.46 | 3.21 | 3 | 8.02 | 0.49 |
| H1.long | miR100.3330 | SV40 | AMELY_V3 | 21.4 | 2.06 | 4 | 3.58 | 0.54 |
| H1.long | miR100.3330 | SV40 | PSG11_V5 | 9.75 | 0.69 | 3 | 8.1 | 2.5 |
| H1.native | miR1.1.14792 | None | AMELY_V3 | 19.49 | 3.84 | 3 | 2.71 | 0.22 |
| H1.native | miR1.1.14792 | None | PSG11_V5 | 13.55 | 8.91 | 3 | 8.81 | 2.01 |
| H1.native | miR1.1.14792 | SV40 | AMELY_V3 | 31.65 | 38.34 | 4 | 3.24 | 0.66 |
| H1.native | miR1.1.14792 | SV40 | PSG11_V5 | 13.65 | 7.94 | 3 | 7.11 | 0.78 |
| H1.native | miR100.3330 | None | AMELY_V3 | 27.76 | 6.87 | 3 | 4.13 | 0.87 |
| H1.native | miR100.3330 | None | PSG11_V5 | 18.38 | 3.6 | 3 | 3.81 | 0.95 |
| H1.native | miR100.3330 | SV40 | AMELY_V3 | 30.61 | 4.02 | 3 | 3.05 | 0.47 |
| H1.native | miR100.3330 | SV40 | PSG11_V5 | 22.41 | 9.68 | 3 | 5.34 | 2.38 |
| H1.short | miR1.1.14792 | None | AMELY_V3 | 13.64 | 1.64 | 2 | 3.19 | 0.3 |
| H1.short | miR1.1.14792 | None | PSG11_V3 | 11.55 | 4.64 | 4 | 2.47 | 0.66 |
| H1.short | miR1.1.14792 | None | PSG11_V5 | 8.99 | 2.36 | 3 | 7.2 | 4.27 |
| H1.short | miR1.1.14792 | SV40 | AMELY_V3 | 11.71 | 1.76 | 3 | 4.35 | 0.36 |
| H1.short | miR1.1.14792 | SV40 | PSG11_V3 | 11.81 | 4.12 | 4 | 4.38 | 1.48 |
| H1.short | miR100.3330 | None | AMELY_V3 | 17.53 | 3.67 | 3 | 4.47 | 1.38 |
| H1.short | miR100.3330 | None | PSG11_V5 | 9.34 | 0.76 | 3 | 6.47 | 0.77 |
| H1.short | miR100.3330 | SV40 | AMELY_V3 | 18.45 | 0.13 | 3 | 2.93 | 0.17 |

### Example 6: Further Evaluation of Production Yields and Truncations of Stuffer Sequences when Combined with ATXN2AmiRNA Expression Cassettes in Different RAAV Vector Formats

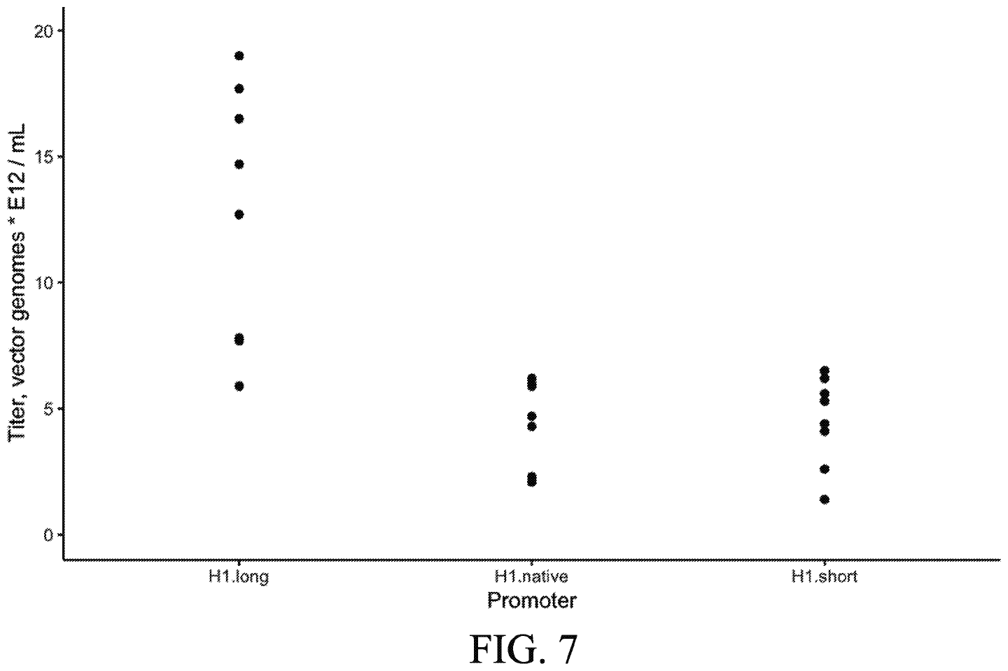

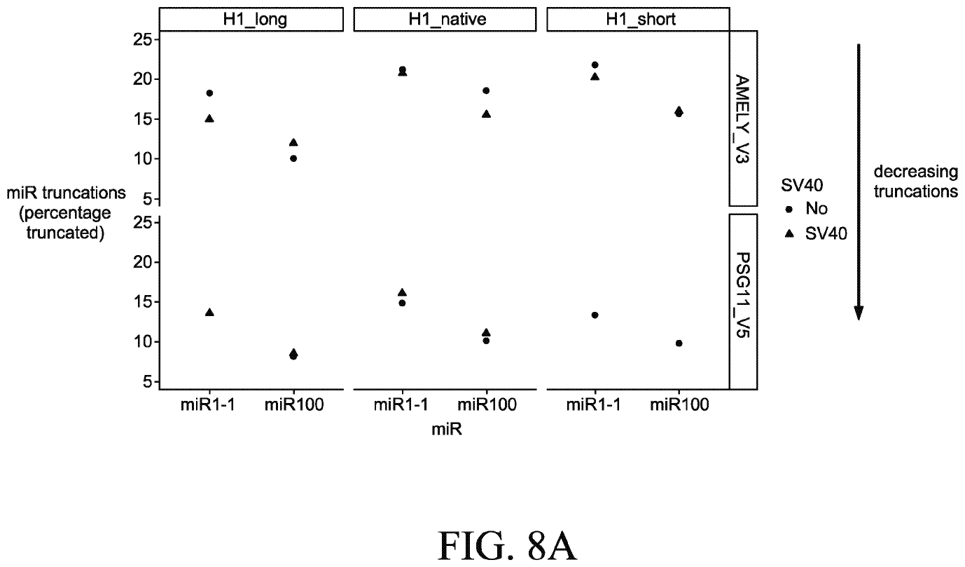

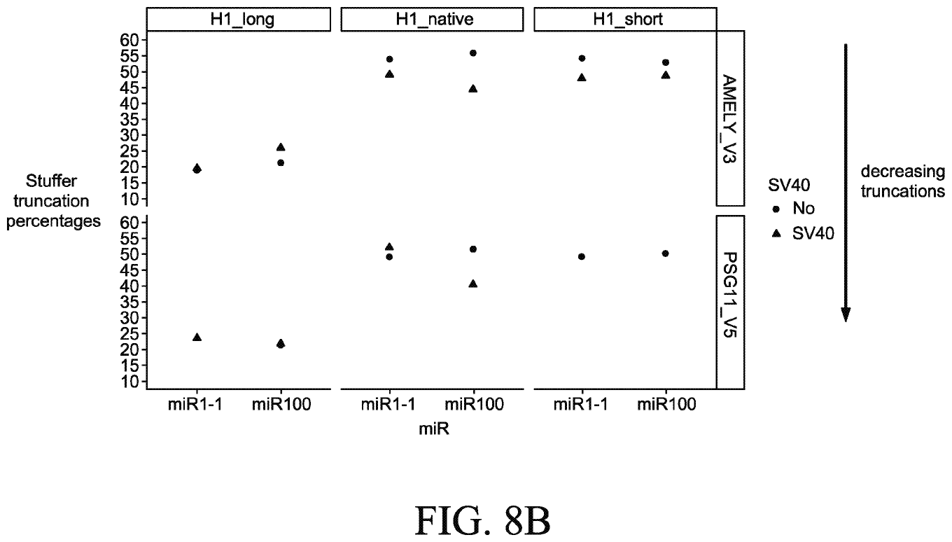

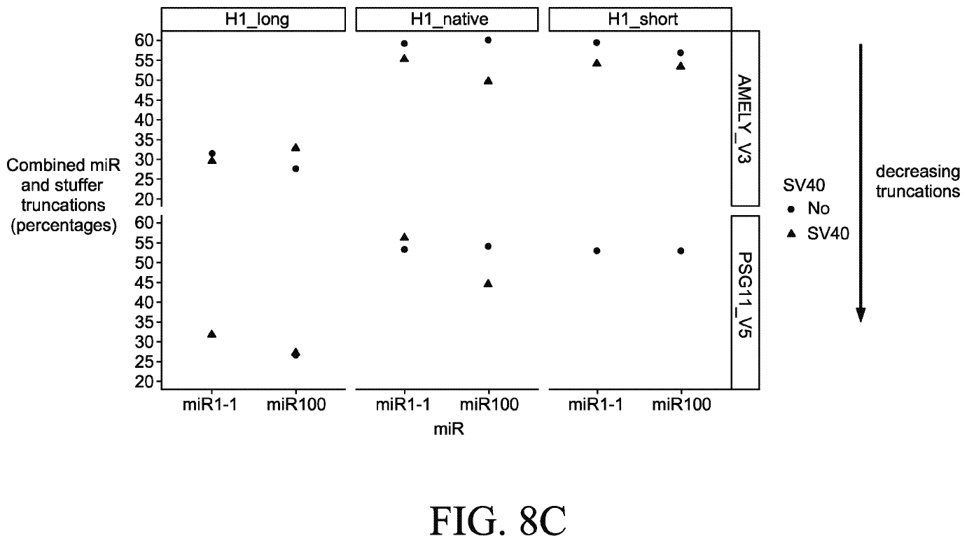

Example 4 shows the unexpected benefit in reducing transcriptional activity downstream of the artificial miRNA sequence when the “H1 long” variant of the promoter was inserted upstream of the PSG11_V5 stuffer sequence versus the “H1 short” variant of the promoter. Production yields of combinations of H1 long and H1 short promoters and the AMELY and PSG11 derived stuffer sequences were also assessed. Production yields were assessed by ddPCR of vector genomes using primer/probe sets specific to the stuffer regions. In general, the use of cis plasmids with H1 long format promoter (SEQ ID NO:52) combinations with the stuffer sequences assessed, which included AMELY_V3 (SEQ ID NO:51), PSG11_V5 (SEQ ID NO:48), PSG11_V3 (SEQ ID NO:47), and PSG11_V2 (SEQ ID NO:46), produced higher production yields (FIG. 7). An analysis of vector truncations (FIGS. 8A-8C) further suggested that there are fewer truncations when the H1 long version of the promoter was included upstream compared to the H1 short promoter. Altogether, there are a number of surprising benefits to the inclusion of the H1 long promoter upstream of these stuffer sequences, perhaps due to local DNA secondary structure elements that might influence rAAV replication and packaging.

### Example 7: Production of Vectors Incorporating Stuffer Sequences with Additional Combinations of Artificial MIRNAS

Stuffer sequences created according to Example 1 and containing combinations of promoter and specific stuffer sequences as described in Example 4 were assessed for their ability, as part of AAV vector genomes, to be packaged in rAAV. The ability to consistently achieve good productivity in the context of different payloads in the vector was also tested for these stuffer sequences.

Stuffer sequence derived from PSG11 intronic region, modified according to the design rules in Example 1, and containing the optimal H1 long promoter variant, was packaged in a set of vectors containing various ATXN2 targeting artificial miRNA packaging cassettes. 5′ ITR to 3′ ITR cis plasmid sequences are set forth in SEQ ID NO:13 (scAAV_H1_long_miR100_1755_PSG11_V5_ITR_to_ITR), SEQ ID NO:14 (scAAV_H1_long_miR100_2586_PSG11_V5_ITR_to_ITR), SEQ ID NO:15 (scAAV_H1_long_miR100_2945_PSG11_V5_ITR_to_ITR), and SEQ ID NO: 16 (scAAV_H1_long_miR100_3330_PSG11_V5_ITR_to_ITR). Sequences for the amiRNA guide, passenger, and expression cassettes are described in Table 5.

TABLE 5
Artificial miRNA Sequences in RNA and
DNA format

| | | | Pas- | |
|---|---|---|---|---|
| ATXN2 | | Guide | senger | |
| Target- | | Se- | Se- | Artificial |
| ing | miR | quence | quence | miRNA |
| Posi- | Back- | (RNA/ | (RNA/ | (RNA/ |
| tion | bone | DNA) | DNA) | DNA) |
| 1755 | miR- | UCGGGUUG | CACACUCC | CCCAAAAGAGAGAAGAUA |
| | 100 | AAAUCUGA | AGCUUUCA | UUGAGGCCUGUUGCCACA |
| | | AGUGUG | AACCGU | UCGGGUUGAAAUCUGAAG |
| | | [SEQ ID | [SEQ ID | UGUGGUAUUAGUCCGCAC |
| | | NO: 1] | NO: 5] | ACUCCAGCUUUCAAACCG |
| | | TCGGGTTG | CACACTCC | UUGUGUCUGUUAGGCAAU |
| | | AAATCTGA | AGCTTTCA | CUCACGGACCUGGGGCUU |
| | | AGTGTG | AACCGT | UGCUUAUAUGCC |
| | | [SEQ ID | [SEQ ID | [SEQ ID NO: 9] |
| | | NO: 59] | NO: 60] | CCCAAAAGAGAGAAGATA |
| | | | | TTGAGGCCTGTTGCCACA |
| | | | | TCGGGTTGAAATCTGAAG |
| | | | | TGTGGTATTAGTCCGCAC |
| | | | | ACTCCAGCTTTCAAACCG |
| | | | | TTGTGTCTGTTAGGCAAT |
| | | | | CTCACGGACCTGGGGCTT |
| | | | | TGCTTATATGCC |
| | | | | [SEQ ID NO: 61] |
| 2945 | miR- | UGUAGUAG | UCAGCCCA | CCCAAAAGAGAGAAGAUA |
| | 100 | AAGGCUUU | AGACUUCU | UUGAGGCCUGUUGCCACA |
| | | GGCUGA | AAUACU | UGUAGUAGAAGGCUUUGG |
| | | [SEQ ID | [SEQ ID | CUGAGUAUUAGUCCGUCA |
| | | NO: 2] | NO: 6] | GCCCAAGACUUCUAAUAC |
| | | TGTAGTAG | TCAGCCCA | UUGUGUCUGUUAGGCAAU |
| | | AAGGCTTT | AGACTTCT | CUCACGGACCUGGGGCUU |
| | | GGCTGA | AATACT | UGCUUAUAUGCC |
| | | [SEQ ID | [SEQ ID | [SEQ ID NO: 10] |
| | | NO: 62] | NO: 63] | CCCAAAAGAGAGAAGATA |
| | | | | TTGAGGCCTGTTGCCACA |
| | | | | TGTAGTAGAAGGCTTTGG |
| | | | | CTGAGTATTAGTCCGTCA |
| | | | | GCCCAAGACTTCTAATAC |
| | | | | TTGTGTCTGTTAGGCAAT |
| | | | | CTCACGGACCTGGGGCTT |
| | | | | TGCTTATATGCC |
| | | | | [SEQ ID NO: 64] |
| 2586 | miR- | UAGAUUCA | CCAAGUCC | CCCAAAAGAGAGAAGAUA |
| | 100 | GAAGUAGA | UAAUUCUG | UUGAGGCCUGUUGCCACA |
| | | ACUUGG | ACUCUU | UAGAUUCAGAAGUAGAAC |
| | | [SEQ ID | [SEQ ID | UUGGGUAUUAGUCCGCCA |
| | | NO: 3] | NO: 7] | AGUCCUAAUUCUGACUCU |
| | | TAGATTCA | CCAAGTCC | UUGUGUCUGUUAGGCAAU |
| | | GAAGTAGA | TAATTCTG | CUCACGGACCUGGGGCUU |
| | | ACTTGG | ACTCTT | UGCUUAUAUGCC |
| | | [SEQ ID | [SEQ ID | [SEQ ID NO: 11] |
| | | NO: 65] | NO: 66] | CCCAAAAGAGAGAAGATA |
| | | | | TTGAGGCCTGTTGCCACA |
| | | | | TAGATTCAGAAGTAGAAC |
| | | | | TTGGGTATTAGTCCGCCA |
| | | | | AGTCCTAATTCTGACTCT |
| | | | | TTGTGTCTGTTAGGCAAT |
| | | | | CTCACGGACCTGGGGCTT |
| | | | | TGCTTATATGCC |
| | | | | [SEQ ID NO: 67] |
| 3330 | miR- | UAUGCUGA | CCACAUCA | CCCAAAAGAGAGAAGAUA |
| | 100 | GACUGAUA | UCCGUCUC | UUGAGGCCUGUUGCCACA |
| | | AUGUGG | AACAUU | UAUGCUGAGACUGAUAAU |
| | | [SEQ ID | [SEQ ID | GUGGGUAUUAGUCCGCCA |
| | | NO: 4] | NO: 8] | CAUCAUCCGUCUCAACAU |
| | | TATGCTGA | CCACATCA | UUGUGUCUGUUAGGCAAU |
| | | GACTGATA | TCCGTCTC | CUCACGGACCUGGGGCUU |
| | | ATGTGG | AACATT | UGCUUAUAUGCC |
| | | [SEQ ID | [SEQ ID | [SEQ ID NO: 12] |
| | | NO: 68] | NO: 69] | CCCAAAAGAGAGAAGATA |
| | | | | TTGAGGCCTGTTGCCACA |
| | | | | TATGCTGAGACTGATAAT |
| | | | | GTGGGTATTAGTCCGCCA |
| | | | | CATCATCCGTCTCAACAT |
| | | | | TTGTGTCTGTTAGGCAAT |
| | | | | CTCACGGACCTGGGGCTT |
| | | | | TGCTTATATGCC |
| | | | | [SEQ ID NO: 70] |
| 1784 | miR- | AUUAACUA | UCCAGACC | CAUGCAGACUGCCUGCUU |
| [also | 1-1 | CUCUUUGG | AAAUAUUA | GGGUACAGACCAAAGAGU |
| re- | | UCUGAA | GUUAAU | AGUCGAAUUAUGGACCUG |
| ferred | | [SEQ ID | [SEQ ID | CUAAGCUAAUUAACUACU |
| to as | | NO: 71] | NO: 72] | CUUUGGUCUGAACUCAGG |
| 14792] | | ATTAACTA | TCCAGACC | CCGGGACCUCUCUCGCCG |
| | | CTCTTTGG | AAATATTA | CACUGAGGGGCACUCCAC |
| | | TCTGAA | GTTAAT | ACCACGGGGGCC |
| | | [SEQ ID | [SEQ ID | [SEQ ID NO: 73] |
| | | NO: 74] | NO: 75] | CATGCAGACTGCCTGCTT |
| | | | | GGGTACAGACCAAAGAGT |
| | | | | AGTCGAATTATGGACCTG |
| | | | | CTAAGCTAATTAACTACT |
| | | | | CTTTGGTCTGAACTCAGG |
| | | | | CCGGGACCTCTCTCGCCG |
| | | | | CACTGAGGGGCACTCCAC |
| | | | | ACCACGGGGGCC |
| | | | | [SEQ ID NO: 76] |

An H1 promoter (“H1 long”) (SEQ ID NO:52 or nucleotides 113-343 of any one of SEQ ID NOS:13-16) was incorporated in the vectors. Yields from AAV production of these vectors are listed in Table 6.

TABLE 6
Production Yield of AAV With H1 long -
PSG11_V5 Stuffer Sequence Containing Vectors

| Vector | Yield | Capsid | Titering method | Production Scale | |
|---|---|---|---|---|---|
| H1-miR100_1755_PSG11_V5 | 6.4E12 vg | AAV9 | ddPCR, probe to | 3180 | cm² |
| (SEQ ID NO: 13) | | | stuffer sequence | | |
| H1-miR100_1755_PSG11_V5 | 4.3E11 vg | AAV-DJ | ddPCR, probe to | 636 | cm² |
| (SEQ ID NO: 13) | | | stuffer sequence | | |
| H1-miR100_2586_PSG11_V5 | 6.6E12 vg | AAV9 | ddPCR, probe to | 3180 | cm² |
| (SEQ ID NO: 14) | | | stuffer sequence | | |
| H1-miR100_2586_PSG11_V5 | 4.7E11 vg | AAV-DJ | ddPCR, probe to | 636 | cm² |
| (SEQ ID NO: 14) | | | stuffer sequence | | |
| H1-miR100_2945_PSG11_V5 | 3.5E12 vg | AAV9 | ddPCR, probe to | 3180 | cm² |
| (SEQ ID NO: 15) | | | stuffer sequence | | |
| H1-miR100_2945_PSG11_V5 | 3.7E11 vg | AAV-DJ | ddPCR, probe to | 636 | cm² |
| (SEQ ID NO: 15) | | | stuffer sequence | | |
| H1-miR100_3330_PSG11_V5 | 5.1E12 vg | AAV9 | ddPCR, probe to | 3180 | cm² |
| (SEQ ID NO: 16) | | | stuffer sequence | | |
| H1-miR100_3330_PSG11 V5 | 4.6E11 vg | AAV-DJ | ddPCR, probe to | 636 | cm² |
| (SEQ ID NO: 16) | | | stuffer sequence | | |

Stuffer sequence derived from AMELY was packaged in several vectors. Vector sequences from 5′ ITR to 3′ ITR that contain an ATXN2 targeting artificial miRNA cassette and an AMELY stuffer sequence are provided for “H1_short_miR16-2-1755_AMELY_V1” (SEQ ID NO: 17) and “H1—miR1-1_1784_AMELY_V3” (SEQ ID NO: 18). In these vectors, the annotation “_V1” or “_V3” for AMELY refers to different versions of the AMELY stuffer sequence, where in “_V3” version ATG sequences are edited and CpG dinucleotides are edited (SEQ ID NO:51 or nucleotides 488-2177 of SEQ ID NO: 18) and “_V1” are not so modified (SEQ ID NO:49 or nucleotides 342-2222 of SEQ ID NO: 17). H1_short refers to a 91 bp truncated form of the H1 promoter (SEQ ID NO:54 or nucleotides 113-203 of SEQ ID NO:17). Yields from productions of these vectors are listed in Table 7.

TABLE 7
Production Yield of AAV With AMELY
Stuffer Sequence Containing Vectors

| | | | Production |
|---|---|---|---|
| Vector | Yield | Titering Method | Scale |
| H1_short_miR16-2- | 0.55E10 | qPCR, probe in stuffer | 3180 cm² |
| 1755_AMELY_V1 | | sequence | |
| [SEQ ID NO: 17] | | | |
| H1_short_miR16-2- | 2.55E11 | qPCR, probe in stuffer | 3180 cm² |
| 3330_AMELY_V1 | | sequence | |
| [SEQ ID NO: 80] | | | |
| H1_long_miR1- | 1.77E12 | ddPCR, probe in stuffer | 3180 cm² |
| 1_1784_AMELY_V3 | | sequence | |
| [SEQ ID NO: 18] | | | |

### Example 8: Demonstration of Functionality of AAV Vector Genomes Packaged Using Stuffer Polynucleotides Combined with Additional AMIRNAS

Experiments were conducted to test the functionality of payloads when incorporated into vector genomes incorporating the stuffer sequences described herein and additional amirnas. This further establishes compatibility of the stuffer sequences with amirna expression cassettes. The vectors described above all express artificial miRNAs targeting ATXN2; functionality is determined by knockdown of the ATXN2 target.

In one experiment, vectors listed in Table 8 were evaluated for knockdown of ATXN2 mRNA in human stem-cell derived motor neurons.—Vectors included scAAV_H1_long_miR100_1755_PSG11_V5_ITR_to_ITR.gb (SEQ ID NO:13), scAAV_H1 long_miR100_2586_PSG11_V5_ITR_to_ITR.gb (SEQ ID NO:14), scAAV_H1_long_miR100_2945_PSG11_V5_ITR_to_ITR.gb (SEQ ID NO:15), and scAAV_H1_long_miR100_3330_PSG11_V5_ITR_to_ITR.gb (SEQ ID NO:16). Stem-cell derived motor neurons were treated with indicated vectors packaged in AAV-DJ, and 7 days after transduction, RNA harvested and assessed for knockdown using RT-ddPCR with probes against ATXN2 and housekeeping probes GUSB and B2M Mean ATXN2 mRNA signal, as normalized against GUSB and B2M probes and against untransduced cells, was measured. Data is listed for cells treated with a dose of 3.16E4 vector genomes/cell.

TABLE 8
ATXN2 Knockdown in amiRNA/AAV Vector Treated
Human Stem-Cell Derived Motor Neurons

| Vector | miR | mean_ATXN2_31600 | SD | N |
|---|---|---|---|---|
| H1-miR- | miR100_1755 | 13.96853215 | 2.13651083 | 6 |
| PSG11_V5 | | | | |
| H1-miR- | miR100_2586 | 29.25180424 | 1.03259645 | 6 |
| PSG11_V5 | | | | |
| H1-miR- | miR100_2945 | 26.83870303 | 2.60157362 | 6 |
| PSG11_V5 | | | | |
| H1-miR- | miR100_3330 | 32.17946149 | 2.69980734 | 6 |
| PSG11_V5 | | | | |

In another experiment, the vector including AMELY_V3 stuffer (SEQ ID NO:51 or nucleotides 488-2177 of SEQ ID NO:18) and an ATXN2 targeting amiRNA under the control of the H1 long promoter was tested by intravenous administration into adult male C57Bl/6 mice and knockdown of the target ATXN2 measured 3 weeks after dosing. Knockdown of Atxn2 was assessed by extracting RNA from liver tissue and performing RT-ddPCR with Atxn2 and the control probes Hprt and Gusb. Table 9 lists average percent knockdown in liver tissue assessed from 3 animals dosed with vector related to animals dosed with vehicle (PBS+0.001% PF-68). The dose administered was on average 8.5E10 in this study, adjusted for mouse weight. As can be seen in the Table 9, there is substantial knockdown in liver from animals dosed with this vector, indicating functionality of the AAV packaged with the stuffer.

TABLE 9
ATXN2 Knockdown in Liver of AAV Vector Treated Mice

| Vector | Atxn2 mRNA, % Vehicle | SD |
|---|---|---|
| H1_long_miR1- | 8.67 | 2.33 |
| 1_1784_AMELY_V3 (SEQ | | |
| ID NO: 18) | | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including U.S. Patent Application No. 63/146,522 filed on Feb. 5, 2021 and PCT Application No. PCT/US2021/016939 filed on Feb. 5, 2021 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A vector stuffer sequence comprising a nucleic acid of about 1300 to about 2300 nucleotides in length and having at least 75% identity to any one of: SEQ ID NOS:45-51; nucleotides 489-2185 of any one of SEQ ID NOS:13-16, nucleotides 342-2222 of SEQ ID NO:17; nucleotides 488-2177 of SEQ ID NO: 18; nucleotides 489-2177 of SEQ ID NO: 19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; and nucleotides 342-2222 of SEQ ID NO:81.

2. The vector stuffer sequence of claim 1, wherein the nucleic acid is about 1500-2000 nucleotides in length.

3. The vector stuffer sequence of claim 1 or 2, wherein the nucleic acid is about 1600 to 1900 nucleotides in length.

4. The vector stuffer sequence of any one of claims 1-3, wherein the nucleic acid has at least 80% identity to any one of: SEQ ID NOS:45-51; nucleotides 489-2185 of any one of SEQ ID NOS:13-16; nucleotides 342-2222 of SEQ ID NO:17; nucleotides 488-2177 of SEQ ID NO: 18; nucleotides 489-2177 of SEQ ID NO: 19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; and nucleotides 349-2229 of SEQ ID NO:78; nucleotides 358-2238 of SEQ ID NO:79; nucleotides 342-2222 of SEQ ID NO:80; and nucleotides 342-2222 of SEQ ID NO:81.

5. The vector stuffer sequence of any one of claims 1-4, wherein the nucleic acid has at least 85% identity to any one of: SEQ ID NOS:45-51; nucleotides 489-2185 of any one of SEQ ID NOS:13-16; nucleotides 342-2222 of SEQ ID NO:17; nucleotides 488-2177 of SEQ ID NO: 18; nucleotides 489-2177 of SEQ ID NO: 19; nucleotides 711-2187 of SEQ ID NO:20; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; and nucleotides 342-2222 of SEQ ID NO:81.

6. The vector stuffer sequence of any one of claims 1-5, wherein the nucleic acid has at least 90% identity to any one of: SEQ ID NOS:45-51; nucleotides 489-2185 of any one of SEQ ID NOS:13-16; nucleotides 342-2222 of SEQ ID NO:17; nucleotides 488-2177 of SEQ ID NO: 18; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; and nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; and nucleotides 342-2222 of SEQ ID NO:81.

7. The vector stuffer sequence of any one of claims 1-6, wherein the nucleic acid has at least 95% identity to any one of: SEQ ID NOS:45-51; nucleotides 489-2185 of any one of SEQ ID NOS:13-16; nucleotides 342-2222 of SEQ ID NO:17; nucleotides 488-2177 of SEQ ID NO:18; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; nucleotides 571-2178 of SEQ ID NO:44; nucleotides 349-2229 of SEQ ID NO:78; nucleotides 358-2238 of SEQ ID NO:79; and nucleotides 342-2222 of SEQ ID NO:80; nucleotides 342-2222 of SEQ ID NO:81.

8. The vector stuffer sequence of any one of claims 1-7, wherein the nucleic acid has at least 97% identity to any one of: SEQ ID NOS:45-51; nucleotides 489-2185 of any one of SEQ ID NOS:13-16; nucleotides 342-2222 of SEQ ID NO:17; nucleotides 488-2177 of SEQ ID NO: 18; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; and nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; and nucleotides 342-2222 of SEQ ID NO:81.

9. The vector stuffer sequence of any one of claims 1-8, wherein the nucleic acid comprises or consists of any one of: SEQ ID NOS:45-51; nucleotides 489-2185 of any one of SEQ ID NOS:13-16; nucleotides 342-2222 of SEQ ID NO: 17; nucleotides 488-2177 of SEQ ID NO: 18; nucleotides 711-2187 of SEQ ID NO:21; nucleotides 488-2177 of SEQ ID NO:22; nucleotides 711-2187 of SEQ ID NO:23; nucleotides 711-2187 of SEQ ID NO:24; nucleotides 252-2132 of SEQ ID NO:25; nucleotides 252-2132 of SEQ ID NO:26; nucleotides 252-2132 of SEQ ID NO:27; nucleotides 252-2132 of SEQ ID NO:28; nucleotides 357-2237 of SEQ ID NO:29; nucleotides 358-2195 of SEQ ID NO:30; nucleotides 580-2187 of SEQ ID NO:31; nucleotides 580-2196 of SEQ ID NO:32; nucleotides 357-2237 of SEQ ID NO:33; nucleotides 358-2195 of SEQ ID NO:34; nucleotides 580-2187 of SEQ ID NO:35; nucleotides 580-2196 of SEQ ID NO:36; nucleotides 348-2228 of SEQ ID NO:37; nucleotides 349-2229 of SEQ ID NO:38; nucleotides 349-2186 of SEQ ID NO:39; nucleotides 571-2178 of SEQ ID NO:40; nucleotides 571-2178 of SEQ ID NO:41; nucleotides 348-2228 of SEQ ID NO:42; nucleotides 349-2186 of SEQ ID NO:43; and nucleotides 571-2178 of SEQ ID NO:44; nucleotides 349-2229 of SEQ ID NO:78; nucleotides 358-2238 of SEQ ID NO:79; nucleotides 342-2222 of SEQ ID NO:80; and nucleotides 342-2222 of SEQ ID NO:81.

10. The vector stuffer sequence of any one of claims 1-9, wherein the vector is an adeno-associated virus (AAV) vector, optionally wherein the AAV vector is self-complementary.

11. The vector stuffer sequence of any one of claims 1-10, wherein the vector stuffer sequence is adjacent to an expression construct comprising a heterologous nucleic acid sequence.

12. The vector stuffer sequence of claim 11, wherein the heterologous nucleic acid sequence encodes a therapeutic agent.

13. The vector stuffer sequence of claim 12, wherein the therapeutic agent comprises a nucleic acid encoding an inhibitory nucleic acid.

14. The vector stuffer sequence of claim 13, wherein the inhibitory nucleic acid comprises a siRNA, miRNA, shRNA, or dsRNA.

15. The vector stuffer sequence of claim 14, wherein the inhibitory nucleic acid comprises an miRNA targeting a neurodegenerative disease related gene, optionally wherein the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease.

16. The vector stuffer sequence of claim 15, wherein the neurodegenerative disease is a polyglutamine repeat disease.

17. The vector stuffer sequence of any one of claim 13-16, wherein the inhibitory nucleic acid targets ATXN2.

18. The vector stuffer sequence of any one of claims 13-17, wherein the heterologous nucleic acid encodes an artificial miRNA comprising:
(a) a guide sequence selected from SEQ ID NOS:1-4 and 71;
(b) a guide sequence provided in SEQ ID NO:1 and a passenger sequence provided by SEQ ID NO:5; a guide sequence provided in SEQ ID NO:2 and a passenger sequence provided by SEQ ID NO:6; a guide sequence provided in SEQ ID NO:3 and a passenger sequence provided by SEQ ID NO:7; a guide sequence provided by SEQ ID NO:4 and a passenger sequence provided by SEQ ID NO:8; or a guide sequence provided by SEQ ID NO:71 and a passenger sequence provided by SEQ ID NO:72; or
(c) a sequence provided by any one of SEQ ID NOs:9-12 and 73.

19. The vector stuffer sequence of any one of claims 11-18, wherein the expression construct comprises a promoter, a polyadenylation signal, a termination signal, or any combination thereof.

20. The vector stuffer sequence of claim 19, wherein the promoter is a H1 promoter, optionally wherein:
(a) the H1 promoter is a H1 long promoter comprising SEQ ID NO:52;nucleotides 113-343 of any one of SEQ ID NOS:13-16 and 18-24;
(b) the H1 promoter is a H1 promoter comprising SEQ ID NO:53; or
(c) the H1 promoter is a H1 short promoter comprising SEQ ID NO:54 or nucleotides 113-203 of any one of SEQ ID NOS:17, 25-28, and 37-44.

21. The vector stuffer sequence of claim 19 or 20, wherein the termination signal is a SV40 termination signal, optionally wherein the SV40 termination signal comprises the sequence provided by SEQ ID NO:77.

22. A recombinant AAV vector comprising the vector stuffer sequence of any one of claims 1-21.

23. The recombinant AAV vector of claim 22, wherein the AAV vector is self-complementary.

24. The recombinant AAV vector of claim 22 or 23, wherein the AAV vector comprises a 5′ inverted terminal repeat (ITR) and a 3′ ITR flanking the expression construct and vector stuffer sequence.

25. The recombinant AAV vector of claim 24, wherein the 5′ ITR and 3′ ITR are obtained from an AAV serotype selected from the group consisting of: AAV1, AAV2, AAV5, AAV6, AAV6.2, AAV7, AAV8, AAV9, AAVRh10, AAV11, and variants thereof.

26. The recombinant AAV vector of claim 24 or 25, wherein one of the 5′ ITR and 3′ ITR lacks a functional terminal resolution site.

27. The recombinant AAV vector of claim 26, wherein the 5′ ITR lacks a functional terminal resolution site.

28. The recombinant AAV vector of any one of claims 24-27, wherein:
(a) the 5′ ITR comprises SEQ ID NO:57 or nucleotides 1-106 of any one of SEQ ID NOS:13-24, 29-44, and 78-82; and/or
(b) the 3′ ITR comprises SEQ ID NO:58; nucleotides 2192-2358 of any one of SEQ ID NOS:13-16; nucleotides 2229-2395 of SEQ ID NO:17; nucleotides 2184-2350 of SEQ ID NO:18; nucleotides 2214-2358 of any one of SEQ ID NOS: 13-16, nucleotides 2251-2395 of SEQ ID NO: 17, nucleotides 2206-2350 of SEQ ID NO:18, nucleotides 2206-2350 of SEQ ID NO:19; nucleotides 2216-2360 of SEQ ID NO:20; nucleotides 2216-2360 of SEQ ID NO:21; nucleotides 2206-2350 of SEQ ID NO:22; nucleotides 2216-2360 of SEQ ID NO:23; nucleotides 2216-2360 of SEQ ID NO:24; nucleotides 2161-2305 of SEQ ID NO:25; nucleotides 2161-2305 of SEQ ID NO:26; nucleotides 2161-2305 of SEQ ID NO:27; nucleotides 2161-2305 of SEQ ID NO:28; nucleotides 2266-2410 of SEQ ID NO:29; nucleotides 2224-2368 of SEQ ID NO:30; nucleotides 2216-2360 of SEQ ID NO:31; nucleotides 2225-2369 of SEQ ID NO:32; nucleotides 2266-2410 of SEQ ID NO:33; nucleotides 2224-2368 of SEQ ID NO:34; nucleotides 2216-2360 of SEQ ID NO:35; nucleotides 2225-2369 of SEQ ID NO:36; nucleotides 2257-2401 of SEQ ID NO:37; nucleotides 2258-2402 of SEQ ID NO:38; nucleotides 2215-2359 of SEQ ID NO:39; nucleotides 2207-2351 of SEQ ID NO:40; nucleotides 2207-2351 of SEQ ID NO:41; nucleotides 2257-2401 of SEQ ID NO:42; nucleotides 2215-2359 of SEQ ID NO:43; nucleotides 2207-2351 of SEQ ID NO:44; nucleotides 2258-2402 of SEQ ID NO:78; nucleotides 2267-2411 of SEQ ID NO:79; nucleotides 2251-2395 of SEQ ID NO:80; nucleotides 2251-2395 of SEQ ID NO:81; or nucleotides 2187-2331 of SEQ ID NO:82.

29. The recombinant AAV vector of any one of claims 22-28, comprising:
(a) the nucleotide sequence of any one of SEQ ID NOS:13-24, 29-44, and 78-80;
(b) the nucleotide sequence of SEQ ID NO: 13 wherein nucleotides 344-481 are substituted with a sequence encoding a miRNA of interest;
(b) the nucleotide sequence of SEQ ID NO:14 wherein nucleotides 344-481 are substituted with a sequence encoding a miRNA of interest;
(c) the nucleotide sequence of SEQ ID NO:15 wherein nucleotides 344-481 are substituted with a sequence encoding a miRNA of interest;
(d) the nucleotide sequence of SEQ ID NO:16 wherein nucleotides 344-481 are substituted with a sequence encoding a miRNA of interest;
(e) the nucleotide sequence of SEQ ID NO:17 wherein nucleotides 204-335 are substituted with a sequence encoding a miRNA of interest;
(f) the nucleotide sequence of SEQ ID NO:18 wherein nucleotides 344-481 are substituted with a sequence encoding a miRNA of interest;
(g) the nucleotide sequence of SEQ ID NO: 19, wherein nucleotides 344-481 are substituted with a sequence encoding a miRNA of interest;
(h) the nucleotide sequence of SEQ ID NO:20, wherein nucleotides 344-481 are substituted with a sequence encoding a miRNA of interest;
(i) the nucleotide sequence of SEQ ID NO:21, wherein nucleotides 344-481 are substituted with a sequence encoding a miRNA of interest;
(j) the nucleotide sequence of SEQ ID NO:22, wherein nucleotides 344-481 are substituted with a sequence encoding a miRNA of interest;
(k) the nucleotide sequence of SEQ ID NO:23, wherein nucleotides 344-481 are substituted with a sequence encoding a miRNA of interest;
(l) the nucleotide sequence of SEQ ID NO:24, wherein nucleotides 344-481 are substituted with a sequence encoding a miRNA of interest;
(m) the nucleotide sequence of SEQ ID NO:29, wherein nucleotides 213-350 are substituted with a sequence encoding a miRNA of interest;
(n) the nucleotide sequence of SEQ ID NO:30, wherein nucleotides 213-350 are substituted with a sequence encoding a miRNA of interest;
(o) the nucleotide sequence of SEQ ID NO:31, wherein nucleotides 213-350 are substituted with a sequence encoding a miRNA of interest;
(p) the nucleotide sequence of SEQ ID NO:32, wherein nucleotides 213-350 are substituted with a sequence encoding a miRNA of interest;
(q) the nucleotide sequence of SEQ ID NO:33, wherein nucleotides 213-350 are substituted with a sequence encoding a miRNA of interest;
(r) the nucleotide sequence of SEQ ID NO:34, wherein nucleotides 213-350 are substituted with a sequence encoding a miRNA of interest;
(s) the nucleotide sequence of SEQ ID NO:35, wherein nucleotides 213-350 are substituted with a sequence encoding a miRNA of interest;
(t) the nucleotide sequence of SEQ ID NO:36, wherein nucleotides 213-350 are substituted with a sequence encoding a miRNA of interest;
(u) the nucleotide sequence of SEQ ID NO:37, wherein nucleotides 204-341 are substituted with a sequence encoding a miRNA of interest;
(v) the nucleotide sequence of SEQ ID NO:38, wherein nucleotides 204-341 are substituted with a sequence encoding a miRNA of interest;
(w) the nucleotide sequence of SEQ ID NO:39, wherein nucleotides 204-341 are substituted with a sequence encoding a miRNA of interest;
(x) the nucleotide sequence of SEQ ID NO:40, wherein nucleotides 204-341 are substituted with a sequence encoding a miRNA of interest;
(y) the nucleotide sequence of SEQ ID NO:41, wherein nucleotides 204-341 are substituted with a sequence encoding a miRNA of interest;
(z) the nucleotide sequence of SEQ ID NO:42, wherein nucleotides 204-341 are substituted with a sequence encoding a miRNA of interest;
(aa) the nucleotide sequence of SEQ ID NO:43, wherein nucleotides 204-341 are substituted with a sequence encoding a miRNA of interest;
(bb) the nucleotide sequence of SEQ ID NO:44, wherein nucleotides 204-341 are substituted with a sequence encoding a miRNA of interest;
(cc) the nucleotide sequence of SEQ ID NO:78, wherein nucleotides 204-342 are substituted with a sequence encoding an artificial miRNA of interest;
(dd) the nucleotide sequence of SEQ ID NO:79, wherein nucleotides 213-351 are substituted with a sequence encoding an artificial miRNA of interest;
(ee) the nucleotide sequence set forth in SEQ ID NO:80, wherein nucleotides 204-335 are substituted with a sequence encoding an artificial miRNA of interest; or
(ff) the nucleotide sequence set forth in SEQ ID NO:81, wherein nucleotides 204-335 are substituted with a sequence encoding an artificial miRNA of interest.

30. A rAAV particle comprising the rAAV vector of any one of claims 22-29.

31. The rAAV particle of claim 30, wherein the rAAV particle comprises a capsid protein.

32. The rAAV particle of claim 31, wherein the capsid protein is capable of crossing the blood-brain barrier.

33. The rAAV particle of claim 31 or 32, wherein the capsid protein is an AAV9 capsid protein.

34. A method of delivering a therapeutic agent to a subject, comprising administering to the subject the rAAV particle of any one of claims 28-32.

35. The method of claim 34, wherein subject has a neurodegenerative disease or is at risk of developing a neurodegenerative disease.

36. The method of claim 34 or 35, wherein the administration comprises direct injection to the CNS of the subject.

37. The method of claim 36, wherein the direct injection is intracerebral injection, intraparenchymal injection, intrathecal injection, intrastriatal injection, subpial injection, or any combination thereof.

38. The method of claim 36, wherein the direct injection is direct injection to the cerebrospinal fluid (CSF) of the subject, optionally wherein the direct injection is intracisternal injection, intraventricular injection, and/or intralumbar injection.

39. The method of any one of claims 35-38, wherein the neurodegenerative disease is spinocerebellar ataxia-2, amyotrophic lateral sclerosis, frontotemporal dementia, primary lateral sclerosis, progressive muscular atrophy, limbic-predominant age-related TDP-43 encephalopathy, chronic traumatic encephalopathy, dementia with Lewy bodies, corticobasal degeneration, progressive supranuclear palsy (PSP), dementia Parkinsonism ALS complex of guam (G-PDC), Pick's disease, hippocampal sclerosis, Huntington's disease, Parkinson's disease, or Alzheimer's disease.


SEQUENCE LISTING (82 sequences)

SEQ ID NO: 1 - RNA, 22 nt, Artificial Sequence
  1755 guide (RNA)
ucggguugaaaucugaagugug

SEQ ID NO: 2 - RNA, 22 nt, Artificial Sequence
  2945 guide (RNA)
uguaguagaaggcuuuggcuga

SEQ ID NO: 3 - RNA, 22 nt, Artificial Sequence
  2586 guide (RNA)
uagauucagaaguagaacuugg

SEQ ID NO: 4 - RNA, 22 nt, Artificial Sequence
  3330 guide (RNA)
uaugcugagacugauaaugugg

SEQ ID NO: 5 - RNA, 22 nt, Artificial Sequence
  1755 passenger (RNA)
cacacuccagcuuucaaaccgu

SEQ ID NO: 6 - RNA, 22 nt, Artificial Sequence
  2945 passenger (RNA)
ucagcccaagacuucuaauacu

SEQ ID NO: 7 - RNA, 22 nt, Artificial Sequence
  2586 passenger (RNA)
ccaaguccuaauucugacucuu

SEQ ID NO: 8 - RNA, 22 nt, Artificial Sequence
  3330 passenger (RNA)
ccacaucauccgucucaacauu

SEQ ID NO: 9 - RNA, 138 nt, Artificial Sequence
  miR100_1755 (RNA)
cccaaaagagagaagauauugaggccuguugccacaucggguugaaaucugaagugugguauuaguccgcacacuccagcuuucaaaccguugugucuguuaggcaaucucacggaccuggggcuuugcuuauaugcc

SEQ ID NO: 10 - RNA, 138 nt, Artificial Sequence
  miR100_2945 (RNA)
cccaaaagagagaagauauugaggccuguugccacauguaguagaaggcuuuggcugaguauuaguccgucagcccaagacuucuaauacuugugucuguuaggcaaucucacggaccuggggcuuugcuuauaugcc

SEQ ID NO: 11 - RNA, 138 nt, Artificial Sequence
  miR100_2586 (RNA)
cccaaaagagagaagauauugaggccuguugccacauagauucagaaguagaacuuggguauuaguccgccaaguccuaauucugacucuuugugucuguuaggcaaucucacggaccuggggcuuugcuuauaugcc

SEQ ID NO: 12 - RNA, 138 nt, Artificial Sequence
  miR100_3330 (RNA)
cccaaaagagagaagauauugaggccuguugccacauaugcugagacugauaauguggguauuaguccgccacaucauccgucucaacauuugugucuguuaggcaaucucacggaccuggggcuuugcuuauaugcc

SEQ ID NO: 13 - DNA, 2358 bp, Artificial Sequence
  scAAV_H1_long_miR100_1755_PSG11_V5_ITR_to_ITR
  misc_feature (1)..(106): 5′ ITR
  misc_feature (113)..(343): H1 long promoter
  misc_feature (344)..(481): miR100_1755
  misc_feature (482)..(488): terminator
  misc_feature (489)..(2185): PSG11_V5
  misc_feature (2214)..(2358): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatcgggttgaaatctgaagtgtggtattagtccgcacactccagctttcaaaccgttgtgtctgttaggcaatctcacggacctggggctttgcttatatgcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 2358 bp, Artificial Sequence
  scAAV_H1_long_miR100_2586_PSG11_V5_ITR_to_ITR
  misc_feature (1)..(106): 5′ ITR
  misc_feature (113)..(343): H1 long promoter
  misc_feature (344)..(481): miR100_2586
  misc_feature (482)..(488): terminator
  misc_feature (489)..(2185): PSG11_V5
  misc_feature (2214)..(2358): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatagattcagaagtagaacttgggtattagtccgccaagtcctaattctgactctttgtgtctgttaggcaatctcacggacctggggctttgcttatatgcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 2358 bp, Artificial Sequence
  scAAV_H1_long_miR100_2945_PSG11_V5_ITR_to_ITR
  misc_feature (1)..(106): 5′ ITR
  misc_feature (113)..(343): H1 long promoter
  misc_feature (344)..(481): miR100_2945
  misc_feature (482)..(488): terminator
  misc_feature (489)..(2185): PSG11_V5
  misc_feature (2214)..(2358): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatgtagtagaaggctttggctgagtattagtccgtcagcccaagacttctaatacttgtgtctgttaggcaatctcacggacctggggctttgcttatatgcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 2358 bp, Artificial Sequence
  scAAV_H1_long_miR100_3330_PSG11_V5_ITR_to_ITR
  misc_feature (1)..(106): 5′ ITR
  misc_feature (113)..(343): H1 long promoter
  misc_feature (344)..(481): miR100_3330
  misc_feature (482)..(488): terminator
  misc_feature (489)..(2185): PSG11_V5
  misc_feature (2214)..(2358): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 2395 bp, Artificial Sequence
  scAAV_H1_short_miR16-2-1755_AMELY V1_ITR_to_ITR
  misc_feature (1)..(106): 5′ ITR
  misc_feature (113)..(203): H1 short promoter
  misc_feature (204)..(335): miR16-2-1755
  misc_feature (336)..(341): terminator
  misc_feature (342)..(2222): AMELY_V1
  misc_feature (2251)..(2395): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacttatgtttggatgaactgacatacttgttccactctcgggttgaaatctgaagtgtgtagtgaaatatatattaaacacacttcaggcttcaacccgttagtgtgacagggatacagcaactattttatcaatttttttattatgcatatcacataattttatttaaaaatcatttagattttgattgcgtgaagtacacaaccagtattccatatcctaatttgttttgtaatacagtcttcaaaagttaaatgcaaagaaattgtcatataaacttcccttcatttggttcaggtttcagtgattgtatatgttcatacatttttattttatcaacgattttaactgatgtgtaaacttcattttttaaaaagatttgacgtagtataatttcagttttctctaaaaagaaatatagtccaaatggtgaatttgtatgagtaaatgagcagttcttaccttattcaagaatactcattcataatatgttgtctcaaaagtttcattcaacacatagcaaaaggaacaattaaataatttaattgtttccagtgagtaccatttacaatacatgtattcaaaccaatgtacaaccacttaagttgaactctataatttccaaagtttctttgttcattcgttgtcattagatattaggtgtccatgaagccttcttctacctttggaacacttaaagcctattattttataacaaaataacaattctttccataaatagctttgtcaaggtgtgagcacattataaatccacggcattttctatgcacagagagaacatgtgctttagactagaatagccaatgatagtcgtcagattacttgggattcaatcaatcaatcaattttcaaaaattctgccccatatcacaattattaagccatttataacatatattatgattttttaattcagaagttatcacatttatacagtagtatatcatcaatagcttgtataaattggaattacaggtggatacatataagtgatagatcacataatcttatatacgtagaaataagattttatggatgtggataaagtttttttattgattattcccattcatttgtaagtgcttcttgtaatgggagattcagtcataaattgaggtaacttctttttaaaagcttaatcatctcaagttagactgaaaactactgaaaatgggaaaccttcgtataggtttttttaaagagatttcccatctacctttcatagcttgttcaaatatatgcactctaaaaattacccaccccatcttatcttaagccttcactgcaattctagcctcttcttgtgagtcaaatctttgaaatgaaagaactgatattcccttattgctgttgcccttaaaattaagagggtttaataatatatttttctattaatgggttgtcataatatacagtctggtagaaattcaataataataaaatttaaacctatcataaaaactgtgttcttaattaccaagccatttagataaatttgtttataatgtaaagatacaacatattacttggactaatagaatcatagatctgtaactactacaactctagaccctgagttgttggaccccttagaactctggggccacctttgctgcaccattttcacaggatttgcattgtagtggcttcacagagagtgactccaaccagagaagcagcagtgttgtctttgccattatggccataggaatattgtatttcaaaaatttgtaactgtgtacacagacatgattttttacaaactaaatttcccattagtgtctgtatgtggagtacacatggtattttcttaaaaatgcctaatattttcagggaattacagatatattagaatccatttgatttaatttactttcatcccatgcagaaattcactgatgcggtaggaaataagaagaaggtaaaactgtttatttccataggtcctgcttttcattgtaagagcaaagcaaacataaaattatgccgcaataatgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 2350 bp, Artificial Sequence
  scAAV_H1_long_miR1-1_1784_AMELY_V3 (5′ ITR .. 3′ ITR)
  misc_feature (1)..(106): 5′ ITR
  misc_feature (113)..(343): H1 long promoter
  misc_feature (344)..(481): miR1-1_1784
  misc_feature (482)..(488): terminator
  misc_feature (488)..(2177): AMELY_V3
  misc_feature (2206)..(2350): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 2350 bp, Artificial Sequence
  scAAV_H1_long_miR1-1_1784_PSG11_V2 (5′ ITR .. 3′ ITR)
  misc_feature (1)..(106): 5′ ITR
  misc_feature (113)..(343): H1 long promoter
  misc_feature (344)..(481): miR1-1_1784
  misc_feature (482)..(488): terminator
  misc_feature (489)..(2177): PSG11_V2
  misc_feature (2206)..(2350): 3′ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcgtcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagacgtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttccggaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacgtgctgtgcccacagcctcaaacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacttgctggtcccaccccaggttgagtgaggcagggccagtcaccagaggagcccggagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacgtgacagaaggggttgaggtggtgacagtgagaagcaactcgttgttcagcactctccttcttgtccctctgtgaagcctcttctaccacaaagggctcagggctgataaagccccctccctacctttctcaggccagacacaaggtcagtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 20 - DNA, 2360 bp, Artificial Sequence
  scAAV_H1_long_miR1-1_1784_SV40_AMELY_V3 (5′ ITR .. 3′ ITR)
  misc_feature (1)..(106): 5′ ITR
  misc_feature (113)..(343): H1 long promoter
  misc_feature (344)..(481): miR1-1_1784
  misc_feature (482)..(488): terminator
  misc_feature (489)..(710): SV40 termination
  misc_feature (711)..(2187): AMELY_V3
  misc_feature (2216)..(2360): 3′ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 21 - DNA, 2360 bp, Artificial Sequence
  scAAV_H1_long_miR1-1_1784_SV40_PSG11_V5 (5′ ITR .. 3′ ITR)
  misc_feature (1)..(106): 5′ ITR
  misc_feature (113)..(343): H1 long promoter
  misc_feature (344)..(481): miR1-1_1784
  misc_feature (482)..(488): terminator
  misc_feature (489)..(710): SV40 termination
  misc_feature (711)..(2187): PSG11_V5
  misc_feature (2216)..(2360): 3′ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 2350 bp, Artificial Sequence
  scAAV_H1_long_miR100_3330_AMELY_V3 (5′ ITR .. 3′ ITR)
  misc_feature (1)..(106): 5′ ITR
  misc_feature (113)..(343): H1 long promoter
  misc_feature (344)..(481): miR100_3330
  misc_feature (482)..(487): terminator
  misc_feature (488)..(2177): AMELY_V3
  misc_feature (2206)..(2350): 3′ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 2360 bp, Artificial Sequence
  scAAV_H1_long_miR100_3330_SV40_AMELY_V3 (5′ ITR .. 3′ ITR)
  misc_feature (1)..(106): 5′ ITR
  misc_feature (113)..(343): H1 long promoter
  misc_feature (344)..(481): miR100_3330
  misc_feature (482)..(488): terminator
  misc_feature (489)..(710): SV40 termination
  misc_feature (711)..(2187): AMELY_V3
  misc_feature (2216)..(2360): 3′ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 2360 bp, Artificial Sequence
  scAAV_H1_long_miR100_3330_SV40_PSG11_V5 (5′ ITR .. 3′ ITR)
  misc_feature (1)..(106): 5′ ITR
  misc_feature (113)..(343): H1 long promoter
  misc_feature (344)..(481): miR100_3330
  misc_feature (482)..(488): terminator
  misc_feature (489)..(710): SV40 termination
  misc_feature (711)..(2187): PSG11_V5
  misc_feature (2216)..(2360): 3′ITR
ctgcgcgctcgctcgctcactgaggccgcccgggcaaagcccgggcgtcgggcgacctttggtcgcccggcctcagtgagcgagcgagcgcgcagagagggagtggacgcgtggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 2305 bp, Artificial Sequence
  scAAV_H1_MCS_AMELY_V1 (5′ ITR .. 3′ ITR)
  misc_feature (1)..(106): 5′ ITR
  misc_feature (113)..(203): H1 short promoter
  misc_feature (204)..(245): multiple cloning site
  misc_feature (246)..(251): terminator
  misc_feature (252)..(2132): AMELY_V1
  misc_feature (2161)..(2305): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacctacataagcaggtgatatgtaaatttcacctgcttaaatagtttttttattatgcatatcacataattttatttaaaaatcatttagattttgattgcgtgaagtacacaaccagtattccatatcctaatttgttttgtaatacagtcttcaaaagttaaatgcaaagaaattgtcatataaacttcccttcatttggttcaggtttcagtgattgtatatgttcatacatttttattttatcaacgattttaactgatgtgtaaacttcattttttaaaaagatttgacgtagtataatttcagttttctctaaaaagaaatatagtccaaatggtgaatttgtatgagtaaatgagcagttcttaccttattcaagaatactcattcataatatgttgtctcaaaagtttcattcaacacatagcaaaaggaacaattaaataatttaattgtttccagtgagtaccatttacaatacatgtattcaaaccaatgtacaaccacttaagttgaactctataatttccaaagtttctttgttcattcgttgtcattagatattaggtgtccatgaagccttcttctacctttggaacacttaaagcctattattttataacaaaataacaattctttccataaatagctttgtcaaggtgtgagcacattataaatccacggcattttctatgcacagagagaacatgtgctttagactagaatagccaatgatagtcgtcagattacttgggattcaatcaatcaatcaattttcaaaaattctgccccatatcacaattattaagccatttataacatatattatgattttttaattcagaagttatcacatttatacagtagtatatcatcaatagcttgtataaattggaattacaggtggatacatataagtgatagatcacataatcttatatacgtagaaataagattttatggatgtggataaagtttttttattgattattcccattcatttgtaagtgcttcttgtaatgggagattcagtcataaattgaggtaacttctttttaaaagcttaatcatctcaagttagactgaaaactactgaaaatgggaaaccttcgtataggtttttttaaagagatttcccatctacctttcatagcttgttcaaatatatgcactctaaaaattacccaccccatcttatcttaagccttcactgcaattctagcctcttcttgtgagtcaaatctttgaaatgaaagaactgatattcccttattgctgttgcccttaaaattaagagggtttaataatatatttttctattaatgggttgtcataatatacagtctggtagaaattcaataataataaaatttaaacctatcataaaaactgtgttcttaattaccaagccatttagataaatttgtttataatgtaaagatacaacatattacttggactaatagaatcatagatctgtaactactacaactctagaccctgagttgttggaccccttagaactctggggccacctttgctgcaccattttcacaggatttgcattgtagtggcttcacagagagtgactccaaccagagaagcagcagtgttgtctttgccattatggccataggaatattgtatttcaaaaatttgtaactgtgtacacagacatgattttttacaaactaaatttcccattagtgtctgtatgtggagtacacatggtattttcttaaaaatgcctaatattttcagggaattacagatatattagaatccatttgatttaatttactttcatcccatgcagaaattcactgatgcggtaggaaataagaagaaggtaaaactgtttatttccataggtcctgcttttcattgtaagagcaaagcaaacataaaattatgccgcaataatgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 2305 bp, Artificial Sequence
  scAAV_H1_MCS_AMELY_V2 (5′ ITR .. 3′ ITR)
  misc_feature (1)..(106): 5′ ITR
  misc_feature (113)..(203): H1 short promoter
  misc_feature (204)..(245): multiple cloning site
  misc_feature (246)..(251): terminator
  misc_feature (252)..(2132): AMELY_V2
  misc_feature (2161)..(2305): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacctacataagcaggtgatatgtaaatttcacctgcttaaatagtttttttattttgcaaatcacaaaattttatttaaaaatcaattagattttgattgcgtgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaacgattttaactgttgtgtaaacttcaatttttaaaaagatttgacgtagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatcgttgtcaatagatattaggtgtccttgaagccttcttctacctttggaacacttaaagcctattattttataacaaaataacaattctttccaaaaatagctttgtcaaggtgtgagcacaatataaatccacggcaatttctttgcacagagagaacttgtgctttagactagaatagccagtgatagtcgtcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacgtagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcgtataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttgcggtaggaaataagaagaaggtaaaactgtttatttccaaaggtcctgcttttcagtgtaagagcaaagcaaacaaaaaattttgccgcaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 27 - DNA, 2305 bp, Artificial Sequence
  scAAV_H1_MCS_PSG11_V1 (5′ ITR .. 3′ ITR)
  misc_feature (1)..(106): 5′ ITR
  misc_feature (113)..(203): H1 short promoter
  misc_feature (204)..(245): multiple cloning site
  misc_feature (246)..(251): terminator
  misc_feature (252)..(2132): PSG11_V1
  misc_feature (2161)..(2305): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacctacataagcaggtgatatgtaaatttcacctgcttaaatagttttttacgaggtcagatgttgcttgtgatgaagggtgtggttaagtctgcagtgcagatggtggaagggatttcactctgtttttagacatagcattggaactaagaattttatgtctatatgggtgaaggggcagggatgtgtaggaagacagttctaattaggatggaggaattatactaggaagtagaggtaaaggaagtgagaaggcttagtaaatagaagaaatgtgaacttatcaaataggctagtgtatacccctggagattcttgattaagtgaattaactagattttagaaaggtaatgaggcagttatatgcaaggaacatttcaaagtctggagacaagaagtcaagcaatactttgagaagtggttgagctttatgggaatggagagaattacactattgagatatgttaaaaataatgaagaaggtttcaccatgtgaaattgtgtttctcatttgaatctgagaaacaaatgagcagaggctggagataattatgtctaaaagacagtggggtacagggagcataaaggccagggagaaagggaactgcaggaattagtgctgagaagcaggagttagtggatgaaggaggagatccaggcccagatacaggcaaataagtcatttccctctcccaagcatggcagtcagccctgcaggaaccaggataagagaaaaatggttggtaaaaactaacataccaggcttgattctttgcagttagctctttatctttctctcccacaagtagtcagtagccttgttctagtgttttatgtgttacctctttttccggaaaaaggaaggtactgttgagttaatgatgatgatagtaatatagtagctgacattggttcctctgtatgtggcatcttgtttcccatctaaattgcacacttagaaacagttaatggtaagccttatattagatatatatagtgtctggtagtcttaccctctctataattacacactttttggcactgcccctttcctgccatgcagagccccaggggtgaatcttcctatttctccagctctgcatcagtcactgtactcagtgctgtgtcccacgtgctgtgcccacagcctcatacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacatgctggtcccaccccaggttgagtgaggcagggccagtcaccagaggagcccggagcagagcaggaagcagagtctgagctgctcctccctcacccaaggggcttcctcctctcatttgggggaaaagtgtgagcttgtttcaaagcctcagatgttccttgtagttcatggaagaggtacaagaaaacaaagacgtgacagaaggggatgaggtggtgacagtgagaagcaactcgatgttcagcactctccttcttgtccctctgtgaagcctcttctaccacatagggctcagggctgataaagccccctccctacctttctcaggccagacacaaggtcagtcatgagaaaacagaaaaaaaaggagaagagagtctgtagagacaaattgggagggttcaggaggagaatttgggatttgcctgtgcccatgggacacaggctgggaataaaaatgttttcctgactcttctctgaaagctagatagactccacctaaaaccctattgccaaggatgctgggcccacagttgatgtcatctgagggaaggggaagctccttgtctcttaaagggacacagtgaccctctgagccaagacacaccctcaagtccgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 2305 bp, Artificial Sequence
  scAAV_H1_MCS_PSG11_V2 (5′ ITR .. 3′ ITR)
  misc_feature (1)..(106): 5′ ITR
  misc_feature (113)..(203): H1 short promoter
  misc_feature (204)..(245): multiple cloning site
  misc_feature (246)..(251): terminator
  misc_feature (252)..(2132): PSG11_V2
  misc_feature (2161)..(2305): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacctacataagcaggtgatatgtaaatttcacctgcttaaatagttttttgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcgtcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagacgtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttccggaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacgtgctgtgcccacagcctcaaacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacttgctggtcccaccccaggttgagtgaggcagggccagtcaccagaggagcccggagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacgtgacagaaggggttgaggtggtgacagtgagaagcaactcgttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggcaaattgttttcctgactcttctctgaaagctagatagactccacctaaaaccctattgccaaggttgctggggagaataaagccacagttgttgtcaactgagggaaggggaagctccttgtctcttaaagggacacagtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 29 - DNA, 2410 bp, Artificial Sequence
  scAAV_H1_native_miR1-1_1784_AMELY_V3 (5′ ITR .. 3′ ITR)
  misc_feature (1)..(106): 5′ ITR
  misc_feature (113)..(212): H1 native promoter
  misc_feature (213)..(350): miR1-1_1784
  misc_feature (351)..(356): terminator
  misc_feature (357)..(2237): AMELY_V3
  misc_feature (2266)..(2410): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 2368 bp, Artificial Sequence
  scAAV_H1_native_miR1-1_1784_PSG11_V5 (5′ ITR .. 3′ ITR)
  misc_feature (1)..(106): 5′ ITR
  misc_feature (113)..(212): H1 native promoter
  misc_feature (213)..(350): miR1-1_1784
  misc_feature (351)..(357): terminator
  misc_feature (358)..(2195): PSG11_V5
  misc_feature (2224)..(2368): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctacccaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccaagggacacaggctgggaatcaaattgttttcctgactcttctctgaaagctagatagactccacctaaaaccctattgccaaggttgctgggccacagttgttgtcaactgagggaaggggaagctccttgtctcttaaagggctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 31 - DNA, 2360 bp, Artificial Sequence
  scAAV_H1_native_miR1-1_1784_SV40_AMELY_V3 (5′ ITR .. 3′ ITR)
  misc_feature (1)..(106): 5′ ITR
  misc_feature (113)..(212): H1 native promoter
  misc_feature (213)..(350): miR1-1_1784
  misc_feature (351)..(357): terminator
  misc_feature (358)..(579): SV40 termination
  misc_feature (580)..(2187): AMELY_V3
  misc_feature (2216)..(2360): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - DNA, 2369 bp, Artificial Sequence
  scAAV_H1_native_miR1-1_1784_SV40_PSG11_V5 (5′ ITR .. 3′ ITR)
  misc_feature (1)..(106): 5′ ITR
  misc_feature (113)..(212): H1 native promoter
  misc_feature (213)..(350): miR1-1_1784
  misc_feature (351)..(357): terminator
  misc_feature (358)..(579): SV40 termination
  misc_feature (580)..(2196): PSG11_V5
  misc_feature (2225)..(2369): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacatgcagactgcctgcttgggtacagaccaaagagtagtcgaattatggacctgctaagctaattaactactctttggtctgaactcaggccgggacctctctcgccgcactgaggggcactccacaccacgggggcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 33 - DNA, 2410 bp, Artificial Sequence
  scAAV_H1_native_miR100_3330_AMELY_V3 (5′ ITR .. 3′ ITR)
  misc_feature (1)..(106): 5′ ITR
  misc_feature (113)..(212): H1 native promoter
  misc_feature (213)..(350): miR100_3330
  misc_feature (351)..(356): terminator
  misc_feature (357)..(2237): AMELY_V3
  misc_feature (2266)..(2410): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 2368 bp, Artificial Sequence
  scAAV_H1_native_miR100_3330_PSG11_V5 (5′ ITR .. 3′ ITR)
  misc_feature (1)..(106): 5′ ITR
  misc_feature (113)..(212): H1 native promoter
  misc_feature (213)..(350): miR100_3330
  misc_feature (351)..(357): terminator
  misc_feature (358)..(2195): PSG11_V5
  misc_feature (2224)..(2368): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctacccaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccaagggacacaggctgggaatcaaattgttttcctgactcttctctgaaagctagatagactccacctaaaaccctattgccaaggttgctgggccacagttgttgtcaactgagggaaggggaagctccttgtctcttaaagggctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 2360 bp, Artificial Sequence
  scAAV_H1_native_miR100_3330_SV40_AMELY_V3 (5′ ITR .. 3′ ITR)
  misc_feature (1)..(106): 5′ ITR
  misc_feature (113)..(212): H1 native promoter
  misc_feature (213)..(350): miR100_3330
  misc_feature (351)..(357): terminator
  misc_feature (358)..(579): SV40 termination
  misc_feature (580)..(2187): AMELY_V3
  misc_feature (2216)..(2360): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 2369 bp, Artificial Sequence
  scAAV_H1_native_miR100_3330_SV40_PSG11_V5 (5′ ITR .. 3′ ITR)
  misc_feature (1)..(106): 5′ ITR
  misc_feature (113)..(212): H1 native promoter
  misc_feature (213)..(350): miR100_3330
  misc_feature (351)..(357): terminator
  misc_feature (358)..(579): SV40 termination
  misc_feature (580)..(2196): PSG11_V5
  misc_feature (2225)..(2369): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccacccaaaagagagaagatattgaggcctgttgccacatatgctgagactgataatgtgggtattagtccgccacatcatccgtctcaacatttgtgtctgttaggcaatctcacggacctggggctttgcttatatgcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 2401 bp, Artificial Sequence
  scAAV_H1_short_miR1-1_1784_AMELY_V3 (5′ ITR .. 3′ ITR)
  misc_feature (1)..(106): 5′ ITR
  misc_feature (113)..(203): H1 short promoter
  misc_feature (204)..(341): miR1-1_1784
  misc_feature (342)..(347): terminator
  misc_feature (348)..(2228): AMELY_V3
  misc_feature (2257)..(2401): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggcctttttttattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 2402 bp, Artificial Sequence
  scAAV_H1_short_miR1-1_1784_PSG11_V3 (5′ ITR .. 3′ ITR)
  misc_feature (1)..(106): 5′ ITR
  misc_feature (113)..(203): H1 short promoter
  misc_feature (204)..(341): miR-1-1 1784
  misc_feature (342)..(348): terminator
  misc_feature (349)..(2229): PSG11_V3
  misc_feature (2258)..(2402): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggcctttttttgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcctcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagaggtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggacccagctcaggagtctgccctgaggttgcttctcttcttatttccttgcagcctggccctggggaggcttggcttgaactggcagctcaatttagccaaattcaggacaggccaccaggactctttctccacacttgctggtcccaccccaggttgagtgaggcagggccagtcaccagaggagccctgagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggcaaattgttttcctgactcttctctgaaagctagatagactccacctaaaaccctattgccaaggttgctggggagaataaagccacagttgttgtcaactgagggaaggggaagctccttgtctcttaaagggacacagtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 2359 bp, Artificial Sequence
  scAAV_H1_short_miR1-1_1784_PSG11_V5 (5′ ITR .. 3′ ITR)
  misc_feature (1)..(106): 5′ ITR
  misc_feature (113)..(203): H1 short promoter
  misc_feature (204)..(341): miR1-1_1784
  misc_feature (342)..(348): terminator
  misc_feature (349)..(2186): PSG11_V5
  misc_feature (2215)..(2359): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctacccaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccaagggacacaggctgggaatcaaattgttttcctgactcttctctgaaagctagatagactccacctaaaaccctattgccaaggttgctgggccacagttgttgtcaactgagggaaggggaagctccttgtctcttaaagggctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 2351 bp, Artificial Sequence
  scAAV_H1_short_miR1-1_1784_SV40_AMELY_V3 (5′ ITR .. 3′ ITR)
  misc_feature (1)..(106): 5′ ITR
  misc_feature (113)..(203): H1 short promoter
  misc_feature (204)..(341): miR1-1_1784
  misc_feature (342)..(348): terminator
  misc_feature (349)..(570): SV40 termination
  misc_feature (571)..(2178): AMELY_V3
  misc_feature (2207)..(2351): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - DNA, 2351 bp, Artificial Sequence
  scAAV_H1_short_miR1-1_1784_SV40_PSG11_V3 (5′ ITR .. 3′ ITR)
  misc_feature (1)..(106): 5′ ITR
  misc_feature (113)..(203): H1 short promoter
  misc_feature (204)..(341): miR1-1_1784
  misc_feature (342)..(348): terminator
  misc_feature (349)..(570): SV40 termination
  misc_feature (571)..(2178): PSG11_V3
  misc_feature (2207)..(2351): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtagtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcctcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagaggtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggacccagctcaggagtctgccctgaggttgcttctcttcttatttccttgcagcctggccctggggaggcttggcttgaactggcagctcaatttagccaaattcaggacaggccaccaggactctttctccacacttgctggtcccaccccaggttgagtgaggcagggccagtcaccagaggagccctgagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggcaaattgttttcctgactcttctctgaaagctagatagactccacctaaaaccctattgccaaggttgctggggagaataaagccacagttgttgtcaactgagggaaggggaagctccttgtctcttaaagggacacagtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 42 - DNA, 2401 bp, Artificial Sequence
  scAAV_H1_short_miR100_3330_AMELY_V3 (5′ ITR .. 3′ ITR)
  misc_feature (1)..(106): 5′ ITR
  misc_feature (113)..(203): H1 short promoter
  misc_feature (204)..(341): miR100_3330
  misc_feature (342)..(347): terminator
  misc_feature (348)..(2228): AMELY_V3
  misc_feature (2257)..(2401): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccccaaaagagagaagatattgaggcctgttgccacatatgctgagactgataatgtgggtattagtccgccacatcatccgtctcaacatttgtgtctgttaggcaatctcacggacctggggctttgcttatatgcctttttttattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 43 - DNA, 2359 bp, Artificial Sequence
  scAAV_H1_short_miR100_3330_PSG11_V5 (5′ ITR .. 3′ ITR)
  misc_feature (1)..(106): 5′ ITR
  misc_feature (113)..(203): H1 short promoter
  misc_feature (204)..(341): miR100_3330
  misc_feature (342)..(348): terminator
  misc_feature (349)..(2186): PSG11_V5
  misc_feature (2215)..(2359): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccccaaaagagagaagatattgaggcctgttgccacatatgctgagactgataatgtgggtattagtccgccacatcatccgtctcaacatttgtgtctgttaggcaatctcacggacctggggctttgcttatatgcctttttttaccaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctacccaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgcccaagggacacaggctgggaatcaaattgttttcctgactcttctctgaaagctagatagactccacctaaaaccctattgccaaggttgctgggccacagttgttgtcaactgagggaaggggaagctccttgtctcttaaagggctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 44 - DNA, 2351 bp, Artificial Sequence
  scAAV_H1_short_miR100_3330_SV40_AMELY_V3 (5′ ITR .. 3′ ITR)
  misc_feature (1)..(106): 5′ ITR
  misc_feature (113)..(203): H1 short promoter
  misc_feature (204)..(341): miR100_3330
  misc_feature (342)..(348): terminator
  misc_feature (349)..(570): SV40 termination
  misc_feature (571)..(2178): AMELY_V3
  misc_feature (2207)..(2351): 3′ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccccaaaagagagaagatattgaggcctgttgccacatatgctgagactgataatgtgggtattagtccgccacatcatccgtctcaacatttgtgtctgttaggcaatctcacggacctggggctttgcttatatgcctttttttcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttggggtaggaaataagaagaaggtaaaactgtttatttccaaaggtcctgcttttcagtgtaagagcaaagcaaacaaaaaattttgccccaatattgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 45 - DNA, 1881 bp, Artificial Sequence
  PSG11_V1 stuffer sequence
acgaggtcagatgttgcttgtgatgaagggtgtggttaagtctgcagtgcagatggtggaagggatttcactctgtttttagacatagcattggaactaagaattttatgtctatatgggtgaaggggcagggatgtgtaggaagacagttctaattaggatggaggaattatactaggaagtagaggtaaaggaagtgagaaggcttagtaaatagaagaaatgtgaacttatcaaataggctagtgtatacccctggagattcttgattaagtgaattaactagattttagaaaggtaatgaggcagttatatgcaaggaacatttcaaagtctggagacaagaagtcaagcaatactttgagaagtggttgagctttatgggaatggagagaattacactattgagatatgttaaaaataatgaagaaggtttcaccatgtgaaattgtgtttctcatttgaatctgagaaacaaatgagcagaggctggagataattatgtctaaaagacagtggggtacagggagcataaaggccagggagaaagggaactgcaggaattagtgctgagaagcaggagttagtggatgaaggaggagatccaggcccagatacaggcaaataagtcatttccctctcccaagcatggcagtcagccctgcaggaaccaggataagagaaaaatggttggtaaaaactaacataccaggcttgattctttgcagttagctctttatctttctctcccacaagtagtcagtagccttgttctagtgttttatgtgttacctctttttccggaaaaaggaaggtactgttgagttaatgatgatgatagtaatatagtagctgacattggttcctctgtatgtggcatcttgtttcccatctaaattgcacacttagaaacagttaatggtaagccttatattagatatatatagtgtctggtagtcttaccctctctataattacacactttttggcactgcccctttcctgccatgcagagccccaggggtgaatcttcctatttctccagctctgcatcagtcactgtactcagtgctgtgtcccacgtgctgtgcccacagcctcatacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacatgctggtcccaccccaggttgagtgaggcagggccagtcaccagaggagcccggagcagagcaggaagcagagtctgagctgctcctccctcacccaaggggcttcctcctctcatttgggggaaaagtgtgagcttgtttcaaagcctcagatgttccttgtagttcatggaagaggtacaagaaaacaaagacgtgacagaaggggatgaggtggtgacagtgagaagcaactcgatgttcagcactctccttcttgtccctctgtgaagcctcttctaccacatagggctcagggctgataaagccccctccctacctttctcaggccagacacaaggtcagtcatgagaaaacagaaaaaaaaggagaagagagtctgtagagacaaattgggagggttcaggaggagaatttgggatttgcctgtgcccatgggacacaggctgggaataaaaatgttttcctgactcttctctgaaagctagatagactccacctaaaaccctattgccaaggatgctgggcccacagttgatgtcatctgagggaaggggaagctccttgtctcttaaagggacacagtgaccctctgagccaagacacaccctcaagtcc

SEQ ID NO: 46 - DNA, 1881 bp, Artificial Sequence
  PSG11_V2 stuffer sequence
gtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcgtcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagacgtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttccggaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacgtgctgtgcccacagcctcaaacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacttgctggtcccaccccaggttgagtgaggcagggccagtcaccagaggagcccggagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacgtgacagaaggggttgaggtggtgacagtgagaagcaactcgttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggcaaattgttttcctgactcttctctgaaagctagatagactccacctaaaaccctattgccaaggttgctggggagaataaagccacagttgttgtcaactgagggaaggggaagctccttgtctcttaaagggacacagt

SEQ ID NO: 47 - DNA, 1881 bp, Artificial Sequence
  PSG11_V3 stuffer sequence
gtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcctcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagaggtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggacccagctcaggagtctgccctgaggttgcttctcttcttatttccttgcagcctggccctggggaggcttggcttgaactggcagctcaatttagccaaattcaggacaggccaccaggactctttctccacacttgctggtcccaccccaggttgagtgaggcagggccagtcaccagaggagccctgagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggcaaattgttttcctgactcttctctgaaagctagatagactccacctaaaaccctattgccaaggttgctggggagaataaagccacagttgttgtcaactgagggaaggggaagctccttgtctcttaaagggacacagt

SEQ ID NO: 48 - DNA, 1697 bp, Artificial Sequence
  PSG11_V5 stuffer sequence
accaggtcagttgttgcttgtgaagaagggtgtggttaagtctgcagtgcagaaggtggaagggatttcactctgtttttagagataggattggaactaagaattttaagtctatttgggtgaaggggcagggaagtgtaggaagacagttctaattaggaaggaggaattatactaggaagtagaggtaaaggaagtgagaaggcttagtaaatagaagaaatctgaacttatcaaataggctagtgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaattgtttggtaaaaactaacaaaccaggcttgattctttgcagttagctctttatctttctctcccacaagtagtcagtagccttgttctagtgttttttgtgttacctctttttcctgaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacctgctgtgcccacagcctcaaacagccagtgacttcagagccaggaaacagctcaggagtctgccctgaggttgcttctcttcttatttccttgcagcctggccaaggggaggcttggcttgaactggcagctcaatttagccaaattcaggacaggccaccaggactctttctccacacttgctggtcccaccccaggttgagtgaggcagggccagtcaccagaggagcccagagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacctgacagaaggggttgaggtggtgacagtgagaagcaactggttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggagggttcaggaggagaatttgggatttgcctgtgccca

SEQ ID NO: 49 - DNA, 1881 bp, Artificial Sequence
  AMELY_V1 stuffer sequence
tattatgcatatcacataattttatttaaaaatcatttagattttgattgcgtgaagtacacaaccagtattccatatcctaatttgttttgtaatacagtcttcaaaagttaaatgcaaagaaattgtcatataaacttcccttcatttggttcaggtttcagtgattgtatatgttcatacatttttattttatcaacgattttaactgatgtgtaaacttcattttttaaaaagatttgacgtagtataatttcagttttctctaaaaagaaatatagtccaaatggtgaatttgtatgagtaaatgagcagttcttaccttattcaagaatactcattcataatatgttgtctcaaaagtttcattcaacacatagcaaaaggaacaattaaataatttaattgtttccagtgagtaccatttacaatacatgtattcaaaccaatgtacaaccacttaagttgaactctataatttccaaagtttctttgttcattcgttgtcattagatattaggtgtccatgaagccttcttctacctttggaacacttaaagcctattattttataacaaaataacaattctttccataaatagctttgtcaaggtgtgagcacattataaatccacggcattttctatgcacagagagaacatgtgctttagactagaatagccaatgatagtcgtcagattacttgggattcaatcaatcaatcaattttcaaaaattctgccccatatcacaattattaagccatttataacatatattatgattttttaattcagaagttatcacatttatacagtagtatatcatcaatagcttgtataaattggaattacaggtggatacatataagtgatagatcacataatcttatatacgtagaaataagattttatggatgtggataaagtttttttattgattattcccattcatttgtaagtgcttcttgtaatgggagattcagtcataaattgaggtaacttctttttaaaagcttaatcatctcaagttagactgaaaactactgaaaatgggaaaccttcgtataggtttttttaaagagatttcccatctacctttcatagcttgttcaaatatatgcactctaaaaattacccaccccatcttatcttaagccttcactgcaattctagcctcttcttgtgagtcaaatctttgaaatgaaagaactgatattcccttattgctgttgcccttaaaattaagagggtttaataatatatttttctattaatgggttgtcataatatacagtctggtagaaattcaataataataaaatttaaacctatcataaaaactgtgttcttaattaccaagccatttagataaatttgtttataatgtaaagatacaacatattacttggactaatagaatcatagatctgtaactactacaactctagaccctgagttgttggaccccttagaactctggggccacctttgctgcaccattttcacaggatttgcattgtagtggcttcacagagagtgactccaaccagagaagcagcagtgttgtctttgccattatggccataggaatattgtatttcaaaaatttgtaactgtgtacacagacatgattttttacaaactaaatttcccattagtgtctgtatgtggagtacacatggtattttcttaaaaatgcctaatattttcagggaattacagatatattagaatccatttgatttaatttactttcatcccatgcagaaattcactgatgcggtaggaaataagaagaaggtaaaactgtttatttccataggtcctgcttttcattgtaagagcaaagcaaacataaaattatgccgcaataatgtga

SEQ ID NO: 50 - DNA, 1881 bp, Artificial Sequence
  AMELY_V2 stuffer sequence
tattttgcaaatcacaaaattttatttaaaaatcaattagattttgattgcgtgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaacgattttaactgttgtgtaaacttcaatttttaaaaagatttgacgtagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatcgttgtcaatagatattaggtgtccttgaagccttcttctacctttggaacacttaaagcctattattttataacaaaataacaattctttccaaaaatagctttgtcaaggtgtgagcacaatataaatccacggcaatttctttgcacagagagaacttgtgctttagactagaatagccagtgatagtcgtcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacgtagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcgtataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtattttcttaaaattgcctaatattttcagggaattacagatatattagaatccaattgatttaatttactttcaacccttgcagaaattcactgttgcggtaggaaataagaagaaggtaaaactgtttatttccaaaggtcctgcttttcagtgtaagagcaaagcaaacaaaaaattttgccgcaatattgtga

SEQ ID NO: 51 - DNA, 1690 bp, Artificial Sequence
  AMELY_V3 stuffer sequence
tattttgcaaatcacaaaattttatttaaaaatcaattagattttgattgcctgaagtacacaaccagtattccaaatcctaatttgttttgtaatacagtcttcaaaagttaattgcaaagaaattgtcaaataaacttcccttcaattggttcaggtttcagtgattgtatttgttcaaacaattttattttatcaaggattttaactgttgtgtaaacttcaatttttaaaaagatttgacctagtataatttcagttttctctaaaaagaaatatagtccaattggtgaatttgtttgagtaattgagcagttcttaccttattcaagaatactcaatcaaaatttgttgtctcaaaagtttcaatcaacacaaagcaaaaggaacaattaaataatttaattgtttccagtgagtaccaattacaatacttgtattcaaaccagtgtacaaccacttaagttgaactctataatttccaaagtttctttgttcaatccttgtcaatagatattaggtgtccttgaagccttcttctacctttggaacacttaaagcctattattttataacaaaataacaattctttccaaaaatagctttgtcaaggtgtgagcacaatataaatccagggcaatttctttgcacagagagaacttgtgctttagactagaatagccagtgatagtcctcagattacttgggattcaatcaatcaatcaattttcaaaaattctgccccaaatcacaattattaagccaattataacaaatattttgattttttaattcagaagttatcacaattatacagtagtatatcaacaatagcttgtataaattggaattacaggtggatacaaataagtgatagatcacaaaatcttatatacctagaaataagattttttggttgtggataaagtttttttattgattattcccaatcaattgtaagtgcttcttgtattgggagattcagtcaaaaattgaggtaacttctttttaaaagcttaatcaactcaagttagactgaaaactactgaaattgggaaaccttcctataggtttttttaaagagatttcccacctacctttcaaagcttgttcaaatatttgcactctaaaaattacccaccccaccttatcttaagccttcactgcaattctagcctcttcttgtgagtcaaatctttgaattgaaagaactgatattcccttattgctgttgcccttaaaattaagagggtttaataatatatttttctattattgggttgtcaaaatatacagtctggtagaaattcaataataataaaatttaaacctatcaaaaaaactgtgttcttaattaccaagccaattagataaatttgtttatattgtaaagatacaacaaattacttggactaatagaatcaaagatctgtaactactacaactctagaccctgagttgttggaccccttagaactctggggccacctttgctgcaccaatttcacaggatttgcagtgtagtggcttcacagagagtgactccaaccagagaagcagcagtgttgtctttgccaatttggccaaaggaatattgtatttcaaaaatttgtaactgtgtacacagacttgattttttacaaactaaatttcccaatagtgtctgtttgtggagtacacttggtatttt

SEQ ID NO: 52 - DNA, 231 bp, Homo sapiens
  misc_feature (1)..(231): H1 long promoter
ggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactctttccca

SEQ ID NO: 53 - DNA, 100 bp, Homo sapiens
  misc_feature (1)..(100): H1 native promoter
atatttgcatgtcgctatgtgttctgggaaatcaccataaacgtgaaatgtctttggatttgggaatcttataagttctgtatgagaccactctttccca

SEQ ID NO: 54 - DNA, 91 bp, Homo sapiens
  misc_feature (1)..(91): H1 short promoter
atatttgcatgtcgctatgtgttctgggaaatcaccataaacgtgaaatgtctttggatttgggaatcttataagttctgtatgagaccac

SEQ ID NO: 55 - DNA, 200 bp, Artificial Sequence
  scAAV_H1_MCS_PSG11_V1
acgaggtcagatgttgcttgtgatgaagggtgtggttaagtctgcagtgcagatggtggaagggatttcactctgtttttagacatagcattggaactaagaattttatgtctatatgggtgaaggggcagggatgtgtaggaagacagttctaattaggatggaggaattatactaggaagtagaggtaaaggaagtga

SEQ ID NO: 56 - DNA, 200 bp, Artificial Sequence
  scAAV_H1_MCS_PSG11_V2
gtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaa

SEQ ID NO: 57 - DNA, 106 bp, Artificial Sequence
  5′ ITR lacking TRS
ctgcgcgctcgctcgctcactgaggccgcccgggcaaagcccgggcgtcgggcgacctttggtcgcccggcctcagtgagcgagcgagcgcgcagagagggagtgg

SEQ ID NO: 58 - DNA, 145 bp, Artificial Sequence
  3′ ITR
taatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 59 - DNA, 22 bp, Artificial Sequence
  1755 guide (DNA)
tcgggttgaaatctgaagtgtg

SEQ ID NO: 60 - DNA, 22 bp, Artificial Sequence
  1755 passenger (DNA)
cacactccagctttcaaaccgt

SEQ ID NO: 61 - DNA, 138 bp, Artificial Sequence
  miR100_1755 (DNA)
cccaaaagagagaagatattgaggcctgttgccacatcgggttgaaatctgaagtgtggtattagtccgcacactccagctttcaaaccgttgtgtctgttaggcaatctcacggacctggggctttgcttatatgcc

SEQ ID NO: 62 - DNA, 22 bp, Artificial Sequence
  2945 guide (DNA)
tgtagtagaaggctttggctga

SEQ ID NO: 63 - DNA, 22 bp, Artificial Sequence
  2945 passenger (DNA)
tcagcccaagacttctaatact

SEQ ID NO: 64 - DNA, 138 bp, Artificial Sequence
  miR100_2945 (DNA)
cccaaaagagagaagatattgaggcctgttgccacatgtagtagaaggctttggctgagtattagtccgtcagcccaagacttctaatacttgtgtctgttaggcaatctcacggacctggggctttgcttatatgcc

SEQ ID NO: 65 - DNA, 22 bp, Artificial Sequence
  2586 guide (DNA)
tagattcagaagtagaacttgg

SEQ ID NO: 66 - DNA, 22 bp, Artificial Sequence
  2586 passenger (DNA)
ccaagtcctaattctgactctt

SEQ ID NO: 67 - DNA, 138 bp, Artificial Sequence
  miR100_2586 (DNA)
cccaaaagagagaagatattgaggcctgttgccacatagattcagaagtagaacttgggtattagtccgccaagtcctaattctgactctttgtgtctgttaggcaatctcacggacctggggctttgcttatatgcc

SEQ ID NO: 68 - DNA, 22 bp, Artificial Sequence
  3330 guide (DNA)
tatgctgagactgataatgtgg

SEQ ID NO: 69 - DNA, 22 bp, Artificial Sequence
  3330 passenger (DNA)
ccacatcatccgtctcaacatt

SEQ ID NO: 70 - DNA, 138 bp, Artificial Sequence
  miR100_3330 (DNA)
cccaaaagagagaagatattgaggcctgttgccacatatgctgagactgataatgtgggtattagtccgccacatcatccgtctcaacatttgtgtctgttaggcaatctcacggacctggggctttgcttatatgcc

SEQ ID NO: 71 - RNA, 22 nt, Artificial Sequence
  1784 (also 14792) guide (RNA)
auuaacuacucuuuggucugaa

SEQ ID NO: 72 - RNA, 22 nt, Artificial Sequence
  1784 (also 14792) passenger (RNA)
uccagaccaaauauuaguuaau

SEQ ID NO: 73 - RNA, 138 nt, Artificial Sequence
  miR1.1_1784 (or miR1.1_14792) (RNA)
caugcagacugccugcuuggguacagaccaaagaguagucgaauuauggaccugcuaagcuaauuaacuacucuuuggucugaacucaggccgggaccucucucgccgcacugaggggcacuccacaccacgggggcc

SEQ ID NO: 74 - DNA, 22 bp, Artificial Sequence
  1784 (also 14792) guide (DNA)
attaactactctttggtctgaa

SEQ ID NO: 75 - DNA, 22 bp, Artificial Sequence
  1784 (also 14792) passenger (DNA)
tccagaccaaatattagttaat

SEQ ID NO: 76 - DNA, 138 bp, Artificial Sequence
  miR1.1_1784 (or miR1.1_14792) (DNA)
catgcagactgcctgcttgggtacagaccaaagagtagtcgaattatggacctgctaagctaattaactactctttggtctgaactcaggccgggacctctctcgccgcactgaggggcactccacaccacgggggcc

SEQ ID NO: 77 - DNA, 222 bp, Artificial Sequence
  SV40 transcription termination signal
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 78 - DNA, 2402 bp, Artificial Sequence
  scAAV9_H1_miR-1-1-XD-14792_PSG11_V1 (5′ ITR .. 3′ ITR)
  misc_feature (1)..(106): 5′ ITR
  misc_feature (113)..(203): H1 short promoter
  misc_feature (204)..(342): miR-1-1_XD-14792
  misc_feature (343)..(348): terminator
  misc_feature (349)..(2229): PSG11_V1
  misc_feature (2258)..(2402): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggccgttttttacgaggtcagatgttgcttgtgatgaagggtgtggttaagtctgcagtgcagatggtggaagggatttcactctgtttttagacatagcattggaactaagaattttatgtctatatgggtgaaggggcagggatgtgtaggaagacagttctaattaggatggaggaattatactaggaagtagaggtaaaggaagtgagaaggcttagtaaatagaagaaatgtgaacttatcaaataggctagtgtatacccctggagattcttgattaagtgaattaactagattttagaaaggtaatgaggcagttatatgcaaggaacatttcaaagtctggagacaagaagtcaagcaatactttgagaagtggttgagctttatgggaatggagagaattacactattgagatatgttaaaaataatgaagaaggtttcaccatgtgaaattgtgtttctcatttgaatctgagaaacaaatgagcagaggctggagataattatgtctaaaagacagtggggtacagggagcataaaggccagggagaaagggaactgcaggaattagtgctgagaagcaggagttagtggatgaaggaggagatccaggcccagatacaggcaaataagtcatttccctctcccaagcatggcagtcagccctgcaggaaccaggataagagaaaaatggttggtaaaaactaacataccaggcttgattctttgcagttagctctttatctttctctcccacaagtagtcagtagccttgttctagtgttttatgtgttacctctttttccggaaaaaggaaggtactgttgagttaatgatgatgatagtaatatagtagctgacattggttcctctgtatgtggcatcttgtttcccatctaaattgcacacttagaaacagttaatggtaagccttatattagatatatatagtgtctggtagtcttaccctctctataattacacactttttggcactgcccctttcctgccatgcagagccccaggggtgaatcttcctatttctccagctctgcatcagtcactgtactcagtgctgtgtcccacgtgctgtgcccacagcctcatacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacatgctggtcccaccccaggttgagtgaggcagggccagtcaccagaggagcccggagcagagcaggaagcagagtctgagctgctcctccctcacccaaggggcttcctcctctcatttgggggaaaagtgtgagcttgtttcaaagcctcagatgttccttgtagttcatggaagaggtacaagaaaacaaagacgtgacagaaggggatgaggtggtgacagtgagaagcaactcgatgttcagcactctccttcttgtccctctgtgaagcctcttctaccacatagggctcagggctgataaagccccctccctacctttctcaggccagacacaaggtcagtcatgagaaaacagaaaaaaaaggagaagagagtctgtagagacaaattgggagggttcaggaggagaatttgggatttgcctgtgcccatgggacacaggctgggaataaaaatgttttcctgactcttctctgaaagctagatagactccacctaaaaccctattgccaaggatgctgggcccacagttgatgtcatctgagggaaggggaagctccttgtctcttaaagggacacagtgaccctctgagccaagacacaccctcaagtccgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 79 - DNA, 2411 bp, Artificial Sequence
  scAAV9_H1_native_miR-1-1-XD-14792_PSG11_V2 (5′ ITR .. 3′ ITR)
  misc_feature (1)..(106): 5′ ITR
  misc_feature (113)..(212): H1 native promoter
  misc_feature (213)..(351): miR1-1-XD-14792
  misc_feature (352)..(357): terminator
  misc_feature (358)..(2238): PSG11_V2
  misc_feature (2267)..(2411): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccactctttcccgcatgcagactgcctgcttgggtacagaccaaagagtagtcgaattatggacctgctaagctaattaactactctttggtctgaactcaggccgggacctctctcgccgcactgaggggcactccacaccacgggggccgttttttgtatacccctggagattcttgattaagtgaattaactagattttagaaaggtattgaggcagttatttgcaaggaacaattcaaagtctggagacaagaagtcaagcaatactttgagaagtggttgagctttttgggattggagagaattacactattgagatttgttaaaaatattgaagaaggtttcaccttgtgaaattgtgtttctcaattgaatctgagaaacaattgagcagaggctggagataattttgtctaaaagacagtggggtacagggagcaaaaaggccagggagaaagggaactgcaggaattagtgctgagaagcaggagttagtggttgaaggaggagatccaggcccagatacaggcaaataagtcaattccctctcccaagcttggcagtcagccctgcaggaaccaggataagagaaaagaagaaaagaaaagaaggattgaaggtgttgttattttacttgggggagcgtcaggaacaagcttgtaacttgagatactgtataattgtttcttcaaggactacaatatttctgttggaaaattgttgggagttgattatattcttgcagttttttttccctctcaccttgtttctcaggtggtgatcagtcctctgtcaattctctactgcactcaggtattttggaaggctttcagacgtgagaaaggctgattgctattttctttgtcagtggaacttgtcacctttgcaccttttcttggttgcaactttttctcagtgtctctgtttggtaaaaactaacaaaccaggcttgattctttgcagttagctctttatctttctctcccacaagtagtcagtagccttgttctagtgttttttgtgttacctctttttccggaaaaaggaaggtactgttgagttattgttgttgatagtaatatagtagctgacagtggttcctctgtttgtggcaacttgtttcccacctaaattgcacacttagaaacagttattggtaagccttatattagatatatatagtgtctggtagtcttaccctctctataattacacactttttggcactgcccctttcctgccttgcagagccccaggggtgaatcttcctatttctccagctctgcaacagtcactgtactcagtgctgtgtcccacgtgctgtgcccacagcctcaaacagccagtgacttcagagccaggacgcagctcaggagtctgccctgaggttgcttctcttcttatttccttgcagcctggcccgggggaggcttggcttgaactggcagctcaatttagccaaattcaggacaggccaccaggactctttctccacacttgctggtcccaccccaggttgagtgaggcagggccagtcaccagaggagcccggagcagagcaggaagcagagtctgagctgctcctccctcacccaaggggcttcctcctctcaattgggggaaaagtgtgagcttgtttcaaagcctcagttgttccttgtagttcttggaagaggtacaagaaaacaaagacgtgacagaaggggttgaggtggtgacagtgagaagcaactcgttgttcagcactctccttcttgtccctctgtgaagcctcttctaccacaaagggctcagggctgataaagccccctccctacctttctcaggccagacacaaggtcagtcttgagaaaacagaaaaaaaaggagaagagagtctgtagagacaaattgggcaaattgttttcctgactcttctctgaaagctagatagactccacctaaaaccctattgccaaggttgctggggagaataaagccacagttgttgtcaactgagggaaggggaagctccttgtctcttaaagggacacagtgctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 80 - DNA, 2395 bp, Artificial Sequence
  H1_short_miR16-2-3330_AMELY_V1 (5′ ITR .. 3′ ITR)
  misc_feature (1)..(106): 5′ ITR
  misc_feature (113)..(203): H1 short promoter
  misc_feature (204)..(335): miR16-2-3330
  misc_feature (336)..(341): terminator
  misc_feature (342)..(2222): AMELY_V1
  misc_feature (2251)..(2395): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacttatgtttggatgaactgacatacttgttccactctatgctgagactgataatgtggtagtgaaatatatattaaaccacattatcgttctcagcatttagtgtgacagggatacagcaactattttatcaatttttttattatgcatatcacataattttatttaaaaatcatttagattttgattgcgtgaagtacacaaccagtattccatatcctaatttgttttgtaatacagtcttcaaaagttaaatgcaaagaaattgtcatataaacttcccttcatttggttcaggtttcagtgattgtatatgttcatacatttttattttatcaacgattttaactgatgtgtaaacttcattttttaaaaagatttgacgtagtataatttcagttttctctaaaaagaaatatagtccaaatggtgaatttgtatgagtaaatgagcagttcttaccttattcaagaatactcattcataatatgttgtctcaaaagtttcattcaacacatagcaaaaggaacaattaaataatttaattgtttccagtgagtaccatttacaatacatgtattcaaaccaatgtacaaccacttaagttgaactctataatttccaaagtttctttgttcattcgttgtcattagatattaggtgtccatgaagccttcttctacctttggaacacttaaagcctattattttataacaaaataacaattctttccataaatagctttgtcaaggtgtgagcacattataaatccacggcattttctatgcacagagagaacatgtgctttagactagaatagccaatgatagtcgtcagattacttgggattcaatcaatcaatcaattttcaaaaattctgccccatatcacaattattaagccatttataacatatattatgattttttaattcagaagttatcacatttatacagtagtatatcatcaatagcttgtataaattggaattacaggtggatacatataagtgatagatcacataatcttatatacgtagaaataagattttatggatgtggataaagtttttttattgattattcccattcatttgtaagtgcttcttgtaatgggagattcagtcataaattgaggtaacttctttttaaaagcttaatcatctcaagttagactgaaaactactgaaaatgggaaaccttcgtataggtttttttaaagagatttcccatctacctttcatagcttgttcaaatatatgcactctaaaaattacccaccccatcttatcttaagccttcactgcaattctagcctcttcttgtgagtcaaatctttgaaatgaaagaactgatattcccttattgctgttgcccttaaaattaagagggtttaataatatatttttctattaatgggttgtcataatatacagtctggtagaaattcaataataataaaatttaaacctatcataaaaactgtgttcttaattaccaagccatttagataaatttgtttataatgtaaagatacaacatattacttggactaatagaatcatagatctgtaactactacaactctagaccctgagttgttggaccccttagaactctggggccacctttgctgcaccattttcacaggatttgcattgtagtggcttcacagagagtgactccaaccagagaagcagcagtgttgtctttgccattatggccataggaatattgtatttcaaaaatttgtaactgtgtacacagacatgattttttacaaactaaatttcccattagtgtctgtatgtggagtacacatggtattttcttaaaaatgcctaatattttcagggaattacagatatattagaatccatttgatttaatttactttcatcccatgcagaaattcactgatgcggtaggaaataagaagaaggtaaaactgtttatttccataggtcctgcttttcattgtaagagcaaagcaaacataaaattatgccgcaataatgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 81 - DNA, 2395 bp, Artificial Sequence
  scAAV_H1_miR16-2-1479_AMELY_V1 (5′ ITR .. 3′ ITR)
  misc_feature (1)..(106): 5′ ITR
  misc_feature (113)..(203): H1 short promoter
  misc_feature (204)..(335): miR16-2-1479
  misc_feature (336)..(341): terminator
  misc_feature (342)..(2222): AMELY_V1
  misc_feature (2251)..(2395): 3′ ITR
ctgcgcgctcgctcgctcactgaggccgcccgggcaaagcccgggcgtcgggcgacctttggtcgcccggcctcagtgagcgagcgagcgcgcagagagggagtggacgcgtatatttgcatgtcgctatgtgttctgggaaatcaccataaacgtgaaatgtctttggatttgggaatcttataagttctgtatgagaccacttatgtttggatgaactgacatacttgttccactcttgctaactggtttgcccttgctagtgaaatatatattaaagcaagggcaagacagttagcattagtgtgacagggatacagcaactattttatcaatttttttattatgcatatcacataattttatttaaaaatcatttagattttgattgcgtgaagtacacaaccagtattccatatcctaatttgttttgtaatacagtcttcaaaagttaaatgcaaagaaattgtcatataaacttcccttcatttggttcaggtttcagtgattgtatatgttcatacatttttattttatcaacgattttaactgatgtgtaaacttcattttttaaaaagatttgacgtagtataatttcagttttctctaaaaagaaatatagtccaaatggtgaatttgtatgagtaaatgagcagttcttaccttattcaagaatactcattcataatatgttgtctcaaaagtttcattcaacacatagcaaaaggaacaattaaataatttaattgtttccagtgagtaccatttacaatacatgtattcaaaccaatgtacaaccacttaagttgaactctataatttccaaagtttctttgttcattcgttgtcattagatattaggtgtccatgaagccttcttctacctttggaacacttaaagcctattattttataacaaaataacaattctttccataaatagctttgtcaaggtgtgagcacattataaatccacggcattttctatgcacagagagaacatgtgctttagactagaatagccaatgatagtcgtcagattacttgggattcaatcaatcaatcaattttcaaaaattctgccccatatcacaattattaagccatttataacatatattatgattttttaattcagaagttatcacatttatacagtagtatatcatcaatagcttgtataaattggaattacaggtggatacatataagtgatagatcacataatcttatatacgtagaaataagattttatggatgtggataaagtttttttattgattattcccattcatttgtaagtgcttcttgtaatgggagattcagtcataaattgaggtaacttctttttaaaagcttaatcatctcaagttagactgaaaactactgaaaatgggaaaccttcgtataggtttttttaaagagatttcccatctacctttcatagcttgttcaaatatatgcactctaaaaattacccaccccatcttatcttaagccttcactgcaattctagcctcttcttgtgagtcaaatctttgaaatgaaagaactgatattcccttattgctgttgcccttaaaattaagagggtttaataatatatttttctattaatgggttgtcataatatacagtctggtagaaattcaataataataaaatttaaacctatcataaaaactgtgttcttaattaccaagccatttagataaatttgtttataatgtaaagatacaacatattacttggactaatagaatcatagatctgtaactactacaactctagaccctgagttgttggaccccttagaactctggggccacctttgctgcaccattttcacaggatttgcattgtagtggcttcacagagagtgactccaaccagagaagcagcagtgttgtctttgccattatggccataggaatattgtatttcaaaaatttgtaactgtgtacacagacatgattttttacaaactaaatttcccattagtgtctgtatgtggagtacacatggtattttcttaaaaatgcctaatattttcagggaattacagatatattagaatccatttgatttaatttactttcatcccatgcagaaattcactgatgcggtaggaaataagaagaaggtaaaactgtttatttccataggtcctgcttttcattgtaagagcaaagcaaacataaaattatgccgcaataatgtgagctagcgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 82 - DNA, 2331 bp, Artificial Sequence
  scAAV_H1_miR1-1-XD-14792_CBh_GFP_SV40p (5′ ITR .. 3′ ITR)
  misc_feature (1)..(106): 5′ ITR
  misc_feature (113)..(203): H1 promoter
  misc_feature (204)..(342): miR1-1-XD-14792
  misc_feature (343)..(348): terminator
  misc_feature (349)..(634): CMV early enhancer contains an 18-bp deletion relative to the standard CMV enhancer
  misc_feature (636)..(913): chicken beta-actin promoter
  misc_feature (983)..(1079): SV40 intron with splice donor and acceptor sites
  misc_feature (1153)..(1869): GFP, mammalian codon-optimized
  misc_feature (1870)..(1899): Myc epitope tag
  misc_feature (1939)..(2161): SV40 termination
  misc_feature (2187)..(2331): 3′ ITR
ctgcgcgctcgctcgctcactgaggccgcccgggcaaagcccgggcgtcgggcgacctttggtcgcccggcctcagtgagcgagcgagcgcgcagagagggagtggctcgagatatttgcatgtcgctatgtgttctgggaaatcaccataaacgtgaaatgtctttggatttgggaatcttataagttctgtatgagaccaccatgcagactgcctgcttgggtacagaccaaagagtagtcgaattatggacctgctaagctaattaactactctttggtctgaactcaggccgggacctctctcgccgcactgaggggcactccacaccacgggggccgttttttcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgttcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagcagaagctgatcagcgaggaggacctgtagcctgcaggggccctcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaccggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag